(19) United States
(12) Patent Application Publication
(10) Pub. No.: US 2012/0295317 A1
(43) Pub. Date: Nov. 22, 2012

(54) Processes and Recombinant Microorganisms for the Production of Cadaverine

(75) Inventors: Hartwig Schröder, Nussloch (DE); Weol Kyu Jeong, Miryongdong Gunsan (KR); Annegret Serwe, Ludwigshafen (DE); Oskar Zelder, Speyer (DE)

(73) Assignee: BASF SE, Ludwigshafen (DE)

(21) Appl. No.: 13/516,034

(22) PCT Filed: Dec. 15, 2010
(86) PCT No.: PCT/EP2010/069802

§ 371 Date: Jun. 14, 2012

Related U.S. Application Data
(60) Provisional application No. 61/287,227, filed on Dec. 17, 2009.

(30) Foreign Application Priority Data
Dec. 17, 2009 (EP) 09179642.5

(51) Int. Cl. C12P 13/00; C07C 209/86; C12N 1/21

(52) U.S. Cl. 435/128; 435/252.32; 435/252.33; 435/253.6; 564/498

(57) ABSTRACT

The present invention relates to the use of recombinant microorganisms comprising DNA molecules in a deregulated form which improve the production of cadaverine, as well as to recombinant DNA molecules and polypeptides used to produce the microorganism, said microorganism comprising an intracellular lysine decarboxylase activity and an enhanced lysine import activity or comprising an intracellular and an extracellular lysine decarboxylase activity or comprising an intracellular and extracellular lysine decarboxylase activity and an enhanced lysine import activity. The present invention also relates to a processes for the production of cadaverine using recombinant microorganisms.

### FIELD OF THE INVENTION

The present invention relates to the use of recombinant microorganisms comprising DNA molecules in a deregulated form which improve the production of cadaverine, as well as to recombinant DNA molecules and polypeptides used to produce the microorganism, said microorganism comprising an intracellular lysine decarboxylase activity and an enhanced lysine import activity or comprising an intracellular and an extracellular lysine decarboxylase activity or comprising an intracellular and extracellular lysine decarboxylase activity and an enhanced lysine import activity. The present invention also relates to a processes for the production of cadaverine using recombinant microorganisms.

### PRIOR ART

JP 2002223770 discloses a method for producing cadaverine by introducing a lysine decarboxylation gene and/or a lysine-cadaverine antiporter gene into a lysine producing microorganism.

JP 2004222569 discloses a method for producing cadaverine by culturing recombinant coryneform bacteria having L-lysine decarboxylase activity and a homoserine auxotrophy.

WO 2007113127 discloses a method for producing cadaverine by culturing recombinant coryneform bacteria having a deregulated L-lysine decarboxylase activity, the detection of a cadaverine acetyltransferase and its deletion to improve cadaverine production.

WO 2008092720 discloses a method for producing cadaverine by fermenting high lysine producing microorganisms expressing an intracellular expressed decarboxylase. Whereby such microorganisms may have a reduced or eliminated expression of a lysine/cadaverine antiporter.

### SUMMARY OF THE INVENTION

In one aspect, the present invention provides a microorganism which has an intracellular lysine decarboxylase activity and an enhanced lysine import activity or has an intracellular and an extracellular lysine decarboxylase activity or has an intracellular and an extracellular lysine decarboxylase activity and an enhanced lysine import activity.

In another aspect, the present invention provides a microorganism as described above and a process for production of cadaverine by cultivating said microorganism as described above, wherein the intracellular lysine decarboxylase activity or the extracellular lysine decarboxylase activity or the intracellular and the extracellular decarboxylase activity of said microorganism is at least partly due to expression of at least one polypeptide being a homolog of a cadA lysine decarboxylase or a IdcC lysine decarboxylase.

In a further aspect, the present invention provides a microorganism having an intracellular lysine decarboxylase activity and an enhanced lysine import activity or has an intracellular and an extracellular lysine decarboxylase activity or has an intracellular and an extracellular lysine decarboxylase activity and an enhanced lysine import activity, wherein the enhanced lysine import activity is due to an enhanced lysine permease activity or an enhanced lysine/cadaverin antiporter activity.

In a further aspect, the present invention provides a microorganism as described above, wherein the enhanced lysine import activity is due to reducing or elimination of the expression of a lysine exporter polypeptide, or due to an enhanced lysine permease activity, or due to an enhanced lysine/cadaverin antiporter activity or any combination thereof.

In a further aspect, the present invention provides a microorganism as described above, wherein the activity of a lysE polypeptide, is decreased or wherein the activity a lysine permease polypeptide is enhanced or wherein the activity of a lysine/cadaverine antiporter polypeptide is enhanced or having any combination of a decreased lysE polypeptide activity, an enhanced activity of a lysine permease polypeptide or an enhanced activity of a lysine/cadaverine antiporter polypeptide.

In a further aspect, the present invention provides a microorganism having any combination of features described above and having no or a decreased acetylcadaverine-forming activity or having no or a decreased aminopropylcadaverine-forming activity or having nor or a decreased homoserine dehydrogenase activity or having no or a decreased lysine degradation activity or wherein at least two of these activities are not present or decreased.

In another aspect the present invention provides a microorganism having any combination of features described above, wherein the activity of a NP_{—}600742 protein or of a spermidine synthase protein or of a homoserine dehydrogenase protein or of a lysine hydroxylase protein or wherein the activity of any combination of these proteins is decreased.

In another aspect the present invention provides a microorganism having a deregulated spermidine forming or uptake activity or a deregulated putrescine forming or uptake activity or any combination thereof.

In another aspect the present invention provides a microorganism having the above described features, wherein the microorganism belongs to the clade Eubacteria, e.g. the genus Corynebacterium or Escherichia, in particular the species Corynebacterium glutamicum or Escherichia coli.

In another aspect the present invention provides a cadaverine production system comprising microorganisms having the above described features and a culture medium containing Lysine.

In another aspect the present invention provides a process for the production of cadaverine comprising fermenting any one of the microorganisms described above in a culture medium comprising Lysine or not comprising Lysine.

In another aspect the present invention provides a process wherein the culture medium comprises at least 0.25 mol/l cadaverine, but not more than 0.1 mol/l of acetylcadaverine or more than 0.1 mol/l of aminopropylcadaverine, or wherein the concentration (mol/l) of cadaverine in the culture medium is at least 1.2 times higher than the percentage (weight/volume) of acetylcadaverine or is at least 1.2 times higher than the percentage (weight/volume) more of aminopropylcadaverine.

In another aspect the present invention provides a culture medium, comprising at least 0.25 mol/cadaverine but not comprising more than 0.1 mol/l of acetylcadaverine or more than 0.1 mol/l of aminopropylcadaverine or a culture medium, wherein the concentration (mol/l) of α-daverine is at least 1.2 times higher than the concentration (mol/l) of acetylcadaverine, or is at least 1.2 times higher than the concentration (mol/l) of aminopropylcadaverine.

In another aspect the present invention provides the use of any one of the microorganism described above for the production of cadaverine, or the use in a process to produce cadaverine.

In another aspect the present invention provides the use of a fermentation broth produced by the fermentation of any one of the microorganisms described above or of a fermentation broth having a high concentration of cadaverine and a low concentration of acetylcadaverine or a low concentration of aminopropylcadaverine for the recovery of cadaverine.

In another aspect the present invention provides a microorganism having the features of converting hemiculllose derived pentoses to fine chemicals such as cadaverine or lysine.

### BRIEF DESCRIPTION OF THE DRAWIN

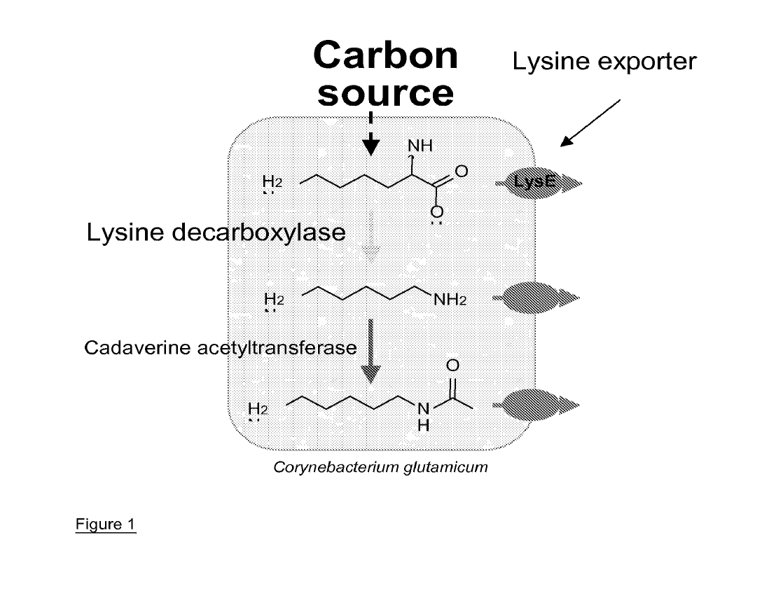

FIG. 1 is a schematic of the DAP biosynthetic pathway in Corynebacterium glutamicum including a cadaverine acetylation step.

### DETAILED DESCRIPTION OF THE INVENTION

In the description that follows, a number of terms are utilized extensively. Definitions are herein provided to facilitate understanding of the invention.

The term cadaverine means 1,5-diaminopentane (CAS-Number: 462-94-2).

The methodologies of the present invention feature recombinant microorganisms, preferably including vectors or genes (e.g., wild-type and/or mutated genes) as described herein and/or cultured in a manner which results in the production of cadaverine.

The term “recombinant” microorganism includes a microorganism (e.g., bacteria, yeast cell, fungal cell, etc.) which has been genetically altered, modified or engineered (e.g., genetically engineered) such that it exhibits an altered, modified or different genotype and/or phenotype (e.g., when the genetic modification affects coding nucleic acid sequences of the microorganism) as compared to the naturally-occurring microorganism from which it was derived.

Promoter. A DNA sequence which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent, if the promoter is a constitutive promoter.

Enhancer. A promoter element. An enhancer can increase the efficiency with which a particular gene is transcribed into mRNA irrespective of the distance or orientation of the enhancer relative to the start site of transcription.

Cloning vector. A DNA molecule, such as a plasmid, cosmid, phagemid, or bacteriophage, which has the capability of replicating autonomously in a host cell and which is used to trans-form cells for gene manipulation. Cloning vectors typically contain one or a small number of restriction endonuclease recognition sites at which foreign DNA sequences may be inserted in a determinable fashion without loss of an essential biological function of the vector, as well as a marker gene which is suitable for use in the identification and selection of cells transformed with the cloning vector. Marker genes typically include genes that provide tetracycline resistance or ampicillin resistance.

Expression vector. A DNA molecule comprising a cloned structural gene encoding a foreign protein which provides the expression of the foreign protein in a recombinant host. Typically, the expression of the cloned gene is placed under the control of (i.e. operably linked to) certain regulatory sequences such as promoter and enhancer sequences. Promoter sequences may be either constitutive or inducible.

Recombinant host. A recombinant host may be any prokaryotic or eukaryotic cell which contains either a cloning vector or an expression vector. This term is also meant to include those prokaryotic or eukaryotic cells that have been genetically engineered to contain the cloned gene(s) in the chromosome or genome of the host cell. For examples of suitable hosts, see Sambrook et al., MOLECULAR CLONING: A LABORATORY MANUAL, Second Edition, Cold Spring Harbor Laboratory, Cold Spring Harbor, N.Y. (1989) [“Sambrook”].

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for, or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

In some embodiments, a microorganism can be physically or environmentally altered to express a gene product at an increased or lower level relative to level of expression of the gene product by the starting microorganism. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

The terms “deregulate,” “deregulated” and “deregulation” refer to alteration or modification of at least one gene in a microorganism, wherein the alteration or modification results in increasing efficiency of cadaverine production in the microorganism relative to cadaverine production in absence of the alteration or modification. In some embodiments, a gene that is altered or modified encodes an enzyme in a biosynthetic pathway or a transport protein, such that the level or activity of the biosynthetic enzyme in the microorganism is altered or modified or that the trans-port specificity or efficiency is altered or modified. In some embodiments, at least one gene that encodes an enzyme in a biosynthetic pathway is altered or modified such that the level or activity of the enzyme is enhanced or increased relative to the level in presence of the unaltered or wild type gene. Deregulation also includes altering the coding region of one or more genes to yield, for example, an enzyme that is feedback resistant or has a higher or lower specific activity. Also, deregulation further encompasses genetic alteration of genes encoding transcriptional factors (e.g., activators, repressors) which regulate expression of genes coding for enzymes or transport proteins.

The terms “overexpress”, “overexpressing”, “overexpressed” and “overexpression” refer to expression of a gene product, in particular to enhancing the expression of a gene product (e.g. a lysine biosynthetic enzyme or sulfate reduction pathway enzyme or cysteine biosynthetic enzyme or a gene or a pathway or a reaction defined and described in this application) at a level greater than that present prior to a genetic alteration of the starting microorganism. In some embodiments, a microorganism can be genetically altered (e.g., genetically engineered) to express a gene product at an increased level relative to that produced by the starting microorganism. Genetic alteration includes, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins). Another way to overexpress a gene product is to enhance the stability of the gene product to increase its life time. Examples for the overexpression of genes in organisms such as C. glutamicum can be found in Eikmanns et al (Gene. (1991) 102, 93-8).

The term “deregulated” includes expression of a gene product (e.g., lysine decarboxylase) at a level lower or higher than that expressed prior to manipulation of the microorganism or in a comparable microorganism which has not been manipulated. In one embodiment, the microorganism can be genetically manipulated (e.g., genetically engineered) to express a level of gene product at a lesser or higher level than that expressed prior to manipulation of the microorganism or in a comparable microorganism which has not been manipulated. Genetic manipulation can include, but is not limited to, altering or modifying regulatory sequences or sites associated with expression of a particular gene (e.g., by removing strong promoters, inducible promoters or multiple promoters), modifying the chromosomal location of a particular gene, altering nucleic acid sequences adjacent to a particular gene such as a ribosome binding site or transcription terminator, decreasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, or other methods to knock-out or block expression of the target protein).

The term “deregulated gene activity”, e.g. deregulated lysine decarboxylase, also means that a gene activity, e.g. a lysine decarboxylase activity, is introduced into a microorganism where the respective gene activity, e.g. the lysine decarboxylase activity, has not been observed before, e.g. by introducing a heterologous gene, e.g. a lysine decarboxylase gene in one or more copies into the microorganism preferably by means of genetic engineering.

The phrase “deregulated pathway or reaction” refers to a biosynthetic pathway or reaction in which at least one gene that encodes an enzyme in a biosynthetic pathway or reaction is altered or modified such that the level or activity of at least one biosynthetic enzyme is altered or modified. The phrase “deregulated pathway” includes a biosynthetic pathway in which more than one gene has been altered or modified, thereby altering level and/or activity of the corresponding gene products/enzymes. In some cases the ability to “deregulate” a pathway (e.g., to simultaneously deregulate more than one gene in a given biosynthetic pathway) in a microorganism arises from the particular phenomenon of microorganisms in which more than one enzyme (e.g., two or three biosynthetic enzymes) are encoded by genes occurring adjacent to one another on a contiguous piece of genetic material termed an “operon.” In other cases, in order to deregulate a pathway, a number of genes must be deregulated in a series of sequential engineering steps.

To express the deregulated genes according to the invention, the DNA sequence encoding the enzyme must be operably linked to regulatory sequences that control transcriptional expression in an expression vector and then, introduced into either a prokaryotic or eukaryotic host cell. In addition to transcriptional regulatory sequences, such as promoters and enhancers, expression vectors can include translational regulatory sequences and a marker gene which is suitable for selection of cells that carry the expression vector.

Suitable promoters for expression in a prokaryotic host can be repressible, constitutive, or inducible. Suitable promoters are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, 1 pp-lac pr, phoA, gal, trc and lacZ promoters of E. coli, the alpha-amylase and the sigma 28-specific promoters of B. subtilis, the promoters of the bacteriophages of Bacillus, Streptomyces promoters, the int promoter of bacteriophage lambda, the bla promoter of the β-lactamase gene of pBR322, and the CAT promoter of the chloramphenicol acetyl transferase gene as well as PGRO, PSOD, PEFTU, PEFTS from Corynebacterium and combinations of these promoters as described in WO2005059144, WO2007011939, WO2007012078, WO2005059093, WO2008049782, WO2006069711, WO2007020295. Prokaryotic promoters are reviewed by Glick, J. Ind. Microbiol. 1:277 (1987); Watson et al., MOLECULAR BIOLOGY OF THE GENE, 4th Ed., Benjamin Cummins (1987); Ausubel et al., supra, and Sambrook et al., supra.

A preferred promoter for the expression of the E. coli lysine decarboxylase is the PsodA, the PGRO and the PEFTU promoter of C. glutamicum described in WO2005059144, WO2007011939, WO2007012078, WO2005059093, WO2008049782, WO2006069711, WO2007020295.

Methods for expressing proteins in prokaryotic hosts are well-known to those of skill in the art. See, for example, Williams et al., “Expression of foreign proteins in E. coli using plasmid vectors and purification of specific polyclonal antibodies,” in DNA CLONING 2: EXPRESSION SYSTEMS, 2nd Edition, Glover et al. (eds.), pages 15-58 (Oxford University Press 1995). Also see, Ward et al., “Genetic Manipulation and Expression of Antibodies,” in MONOCLONAL ANTIBODIES: PRINCIPLES AND APPLICATIONS, pages 137-185 (Wiley-Liss, Inc. 1995); and Georgiou, “Expression of Proteins in Bacteria,” in PROTEIN ENGINEERING: PRINCIPLES AND PRACTICE, Cleland et al. (eds.), pages 101-127 (John Wiley & Sons, Inc. 1996). Further methods to increase or decrease gene expression and protein production can be found in WO2008049782.

An expression vector can be introduced into bacterial host cells using a variety of techniques including calcium chloride transformation, electroporation, and the like. See, for example, Ausubel et al. (eds.), SHORT PROTOCOLS IN MOLECULAR BIOLOGY, 3rd Edition, pages 1-1 to 1-24 (John Wiley & Sons, Inc. 1995).

As used herein, a substantially pure protein means that the desired purified protein is essentially free from contaminating cellular components, as evidenced by a single band following polyacrylamide-sodium dodecyl sulfate gel electrophoresis (SDS-PAGE). The term “substantially pure” is further meant to describe a molecule which is homogeneous by one or more purity or homogeneity characteristics used by those of skill in the art. For example, a substantially pure lysine decarboxylase will show constant and reproducible characteristics within standard experimental deviations for parameters such as the following: molecular weight, chromatographic migration, amino acid composition, amino acid sequence, blocked or unblocked N-terminus, HPLC elution profile, biological activity, and other such parameters. The term, however, is not meant to exclude artificial or synthetic mixtures of lysine decarboxylase with other compounds.

In addition, the term is not meant to exclude lysine decarboxylase fusion proteins isolated from a recombinant host.

In a first aspect, the present invention provides a microorganism having an intracellular lysine decarboxylase activity and an enhanced lysine import activity or comprising an intracellular and an extracellular lysine decarboxylase activity or comprising an intracellular and an extracellular lysine decarboxylase activity and an enhanced lysine import activity

The microorganism can be any prokaryotic or eukaryotic microorganism, in particular bacteria, archaea, yeasts and fungi. Preferred are microorganisms being selected from the genus of Corynebacterium with a particular focus on Corynebacterium glutamicum, the genus of Escherichia with a particular focus on Escherichia coli, the genus of Bacillus, particularly Bacillus subtilis, and the genus of Streptomyces.

As set out above, a preferred embodiment of the invention relates to the use of host cells which are selected from coryneform bacteria such as bacteria of the genus Corynebacterium. Particularly preferred are the species Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola and Corynebacterium effiziens. Other preferred embodiments of the invention relate to the use of Brevibacteria and particularly the species Brevibacterium flavum, Brevibacterium lactofermentum and Brevibacterium divarecatum.

In other preferred embodiments of the invention the host cells may be selected from the group comprising Corynebacterium glutamicum ATCC13032, C. acetoglutamicum ATCC15806, C. acetoacidophilum ATCC13870, Corynebacterium thermoaminogenes FERMBP-1539, Corynebacterium melassecola ATCC17965, Corynebacterium effiziens DSM 44547, Corynebacterium effiziens DSM 44549, Brevibacterium flavum ATCC14067, Brevibacterium lactoformentum ATCC13869, Brevibacterium divarecatum ATCC 14020, Corynebacterium glutamicum KFCC10065 and Corynebacterium glutamicum ATCC21608 as well as strains that are derived thereof by e.g. classical mutagenesis and selection or by directed mutagenesis.

Other particularly preferred strains of C. glutamicum may be selected from the group comprising ATCC13058, ATCC13059, ATCC13060, ATCC21492, ATCC21513, ATCC21526, ATCC21543, ATCC13287, ATCC21851, ATCC21253, ATCC21514, ATCC21516, ATCC21299, ATCC21300, ATCC39684, ATCC21488, ATCC21649, ATCC21650, ATCC19223, ATCC13869, ATCC21157, ATCC21158, ATCC21159, ATCC21355, ATCC31808, ATCC21674, ATCC21562, ATCC21563, ATCC21564, ATCC21565, ATCC21566, ATCC21567, ATCC21568, ATCC21569, ATCC21570, ATCC21571, ATCC21572, ATCC21573, ATCC21579, ATCC19049, ATCC19050, ATCC19051, ATCC19052, ATCC19053, ATCC19054, ATCC19055, ATCC19056, ATCC19057, ATCC19058, ATCC19059, ATCC19060, ATCC19185, ATCC13286, ATCC21515, ATCC21527, ATCC21544, ATCC21492, NRRL B8183, NRRL W8182, B12NRRLB12416, NRRLB12417, NRRLB12418 and NRRLB11476.

The abbreviation KFCC stands for Korean Federation of Culture Collection, ATCC stands for American-Type Strain Culture Collection and the abbreviation DSM stands for Deutsche Sammlung von Mikroorganismen. The abbreviation NRRL stands for ARS cultures collection Northern Regional Research Laboratory, Peorea, Ill., USA.

In certain embodiments, a microorganism of the invention is a “Campbell in” or “Campbell out” microorganism (or cell or transformant). As used herein, the phrase “Campbell in” transformant shall mean a transformant of an original host cell in which an entire circular double stranded DNA molecule (for example a plasmid) has integrated into a chromosome of the cell by a single homologous recombination event (a cross in event), and which effectively results in the insertion of a linearized version of the circular DNA molecule into a first DNA sequence of the chromosome that is homologous to a first DNA sequence of the circular DNA molecule. The phrase “Campbelled in” refers to the linearized DNA sequence that has been integrated into the chromosome of the “Campbell in” transformant. A “Campbell in” transformant contains a duplication of the first homologous DNA sequence, that includes and surrounds the homologous recombination crossover point.

“Campbell out” refers a cell descended from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of the linearized insert, the second recombination event resulting in the deletion (jettisoning) of a portion of the integrated DNA sequence, but, importantly, also resulting in a portion (this can be as little as a single base) of the integrated DNA sequence remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, and so on.

The homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and second homologous DNA sequence are usually separated by a region of partial non-homology, and it is this region of non-homology that remains deposited in the chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. A preferred length for the first and second homologous sequences is about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs.

Lysine decarboxylase activity refers to the decarboxylation of L-lysine into cadaverine, which is catalyzed by a Lysine decarboxylase. The enzyme has been classified as E.C. 4.1.1.18. For example, enzymes isolated from Escherichia coli having lysine decarboxylase activity are the cadA gene product (Kyoto Encyclopedia of Genes and Genomes, Entry b4131) and the IdcC gene product (Kyoto Encyclopedia of Genes and Genomes, Entry JW0181).

Methods to measure and compare enzymatic activities are known in the art and are described for example in “Handbook of Corynebacetrium glutamicum 2005 Eggeling, Borth eds. CRC Press Boca Raton USA and references within) or in Methods in Enzymology Volume 17, Part 1 pp. 3-1098 (1970), Volume 17, Part 2, pp. 3-961 (1971) Volume 41, pp. 3-564 (1975), Volume 42 pp. 3-537 (1975), Volume 63, pp. 3-547 (1979), Volume 64, pp. 3-418 (1980), Volume 89, pp. 3-656 (1982), Volume 90 pp. 3-602 (1982), Volume 142, pp. 3-732 (1987), Volume 143 pp. 3-582 (1987) and references therein.”. A standard strain used for comparison of Corynebacterium glutamicum strains is ATCC13032 (Handbook of Corynebacetrium glutamicum 2005 Eggeling, Borth eds. CRC Press Boca Raton USA) The amino acid sequences of E. coli IdcC is disclosed in accession number SEQ ID NO: 1 and of E. coli cadA is disclosed in SEQ ID NO: 2.

DNA molecules encoding the E. coli lysine decarboxylase gene can be obtained by screening cDNA or genomic libraries with polynucleotide probes having nucleotide sequences reversetranslated from the amino acid sequence of SEQ ID NO: 1 or 2.

Alternatively, the E. coli lysine decarboxylase genes or any genes described herein can be obtained by synthesizing DNA molecules using mutually priming long oligonucleotides. See, for example, Ausubel et al., (eds.), CURRENT PROTOCOLS IN MOLECULAR BIOLOGY, pages 8.2.8 to 8.2.13 (1990) [“Ausubel”]. Also, see Wosnick et al., Gene 60:115 (1987); and Ausubel et al. (eds.), SHORT PROTOCOLS IN MOLECULAR BIOLOGY, 3rd Edition, pages 8-8 to 8-9 (John Wiley & Sons, Inc. 1995). Established techniques using the polymerase chain reaction provide the ability to synthesize DNA molecules at least 2 kilobases in length. Adang et al., Plant Molec. Biol. 21:1131 (1993); Bambot et al., PCR Methods and Applications 2:266 (1993); Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263-268, (Humana Press, Inc. 1993); Holowachuk et al., PCR Methods Appl. 4:299 (1995).

Variants of E. coli lysine decarboxylase or variants of any gene described herein can be produced that contain conservative amino acid changes, compared with the parent enzyme. That is, variants can be obtained that contain one or more amino acid substitutions of e.g. SEQ ID NO: 1 or 2, in which an alkyl amino acid is substituted for an alkyl amino acid in the lysine decarboxylase amino acid sequence, an aromatic amino acid is substituted for an aromatic amino acid in the E. coli lysine decarboxylase amino acid sequence, a sulfur-containing amino acid is substituted for a sulfur-containing amino acid in the E. coli lysine decarboxylase amino acid sequence, a hydroxy-containing amino acid is substituted for a hydroxy-containing amino acid in the E. coli lysine decarboxylase amino acid sequence, an acidic amino acid is substituted for an acidic amino acid in the E. coli lysine decarboxylase amino acid sequence, a basic amino acid is substituted for a basic amino acid in the E. coli lysine decarboxylase amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) cysteine and methionine, (4) serine and threonine, (5) aspartate and glutamate, (6) glutamine and asparagine, and (7) lysine, arginine and histidine.

Conservative amino acid changes in the E. coli lysine decarboxylase can be introduced by substituting nucleotides for the nucleotides recited in SEQ ID NO:1 or 2. Such “conservative amino acid” variants can be obtained, for example, by oligonucleotide-directed mutagenesis, linkerscanning mutagenesis, mutagenesis using the polymerase chain reaction, and the like. Ausubel et al., supra, at pages 8.0.3-8.5.9; Ausubel et al. (eds.), SHORT PROTOCOLS IN MOLECULAR BIOLOGY, 3rd Edition, pages 8-10 to 8-22 (John Wiley & Sons, Inc. 1995). Also see generally, McPherson (ed.), DIRECTED MUTAGENESIS: A PRACTICAL APPROACH, IRL Press (1991). The ability of such variants to convert L-lysine to cadaverine can be determined using a standard enzyme activity assay, such as the assay described herein.

Preferred lysine decarboxylases according to the invention are the lysine decarboxylase from E. coli and their equivalent genes, which have up to 80%, preferably 90% and most preferred 95% and 98% sequence identity (based on amino acid sequence) with the corresponding “original” gene product and have still the biological activity of lysine decarboxylase. These equivalent genes can be easily constructed by introducing nucleotide substitutions, deletions or insertions by methods known in the art or by cloning homolog genes of other organisms, which can be identified and cloned according to methods well known in the art e.g. database searches, library screenings, complementation assays or enzymatic activity tests. Preferably the nucleotide sequence of cloned homolog genes is optimized for expression in the intended host microorganism e.g. by adapted to the codon usage of Corynebacterium glutamicum or Escherichia coli.

Other preferred embodiments of the invention use the lysine decarboxylases of E. coli (SEQ ID NO: 1 or 2) which are retranslated into DNA by applying the codon usage of Corynebacterium glutamicum. These lysine decarboxylase polynucleotide sequences are useful for expression of lysine decarboxylase in microorganism of the genus Corynebacterium, especially Corynebacterium glutamicum.

An intracellular lysine decarboxylase activity is brought about by a lysine decarboxylase located on the cytoplasmic side of the cellmembrane of the microorganism. In case of an eukaryotic microorganism the lysine decarboxylase may be located in one or more compartments of the cell. Preferably it is located in the cytoplasm of the microorganism, inserted or attached to the cellmembrane.

An extracellular lysine decarboxylase activity is brought about by a lysine decarboxylase located outside of the cell membrane. The lysine decarboxylase may be located in the periplasmic space, in the cell wall, on the surface of the cell wall, secreted into the medium, or any combination of those.

A microorganism of the invention comprises an intracellular lysine decarboxylase activity. preferably a high intracellular decarboxylase activity. An intracellular lysine decarboxylase activity can be created or enhanced by transforming the microorganism with one or more lysine decarboxylase genes to be expressed by the microorganism. Additionally or alternatively lysine decarboxylase genes can be mutated to enhance expression or the enzymatic activity of the encoded lysine decarboxylase.

In another embodiment, the intracellular lysine decarboxylase activity is combined with an extracellular lysine decarboxylase activity. A microorganism lacking either an intracellular or extracellular lysine decarboxylase activity may be transformed to express a lysine decarboxylase either intracellular or extracellular. Extracellular expression can be achieved by mutating a lysine decarboxylase gene in order to comprise signal sequences for extracellular expression, e.g. secretion signals or signals for molecular anchors, which are functional in the microorganism to be transformed. Examples for extracellular expression can be found in Choi and Lee Appl Microbiol Biotechnol 2004 64: 625-635, Current Opinion in Biotechnology 2005, 16:538-545, Trends in Biotechnology 2007 16 73-79.

In case the intracellular or extracellular lysine decarboxylase activity is due to expression of more than one lysine decarboxylase genes coding for different polypeptides having lysine decarboxylase activity, the total intracellular or extracellular lysine decarboxylase activity will be the result of those different lysine decarboxylase activities. The contribution of a particular lysine decarboxylase gene to the total lysine decarboxylase activity can be measured by comparing the expression level of different lysine decarboxylase genes in a particular microorganism and comparing the specific enzymatic activities of the lysine decarboxylases expressed from these genes. The expression level of different genes can be measured according to methods well known in the art, preferably the expression level is measured on the protein level, e.g. by Western Blots or ELISA assays. Methods to measure the specific enzymatic activity of a lysine decarboxylase are also well known in the art. A preferred method is disclosed in WO2007113127.

In case a endogenous lysine decarboxylase activity is enhanced by transgenic expression of at least one further polypeptide having lysine decarboxylase activity, the contribution of this or those additional polypeptides can be measured by comparing the total lysine decarboxylase activity of the transformed and untransformed microorganism.

In a preferred embodiment the intracellular or the extracellular decarboxylase activity or both activities are at least partly, preferably to more than 30% or to more than 50% or to more than 60% or to more than 70% or to more than 75%, 80%, 85%, 90%, 95% 98%, 99% due to one or more polypeptides comprising an amino acid sequence being at least 80% or at least 85% or at least 90% or at least 95% or at least 98% identical to SEQ ID NO: 1 or 2 and having lysine decarboxylase activity, preferably having a high lysine decarboxylase activity.

In one embodiment the intracellular or the extracellular decarboxylase activity or both activities are to more than 98% or to more than 99% due to one or more polypeptides comprising an amino acid sequence being at least 80% or at least 85% or at least 90% or at least 95% or at least 98% identical to SEQ ID NO: 1 or 2 and having lysine decarboxylase activity, preferably having a high lysine decarboxylase activity.

In one embodiment the intracellular or the extracellular decarboxylase activity or both activities are at least partly, preferably to more than 30% or to more than 50% or to more than 60% or to more than 70% or to more than 75%, 80%, 85%, 90%, 95% 98%, 99% due to one or more polypeptides comprising an amino acid sequence being at least 80% or at least 85% or at least 90% or at least 95% or at least 98% identical to SEQ ID NO: 1 and having lysine decarboxylase activity, preferably having a high lysine decarboxylase activity.

In one embodiment the intracellular or the extracellular decarboxylase activity or both activities are at least partly, preferably to more than 30% or to more than 50% or to more than 60% or to more than 70% or to more than 75%, 80%, 85%, 90%, 95% 98%, 99% due to one or more polypeptides comprising an amino acid sequence being at least 80% or at least 85% or at least 90% or at least 95% or at least 98% identical to SEQ ID NO: 2 and having lysine decarboxylase activity, preferably having a high lysine decarboxylase activity.

In one embodiment, the microorganisms comprising an intracellular lysine decarboxylase activity or an intracellular and an extracellular lysine decarboxylase activity does also comprise an enhanced lysine import capacity.

In improved lysine import capacity, can be achieved by any measure, which enhances the flow of lysine from the fermentation medium into the microorganism or by reducing the flow of lysine from microorganism to the fermentation medium.

Methods to determine the lysine export and import activity can be found in Bellmann, A, Vrljic, M, Patek, M, et al. MICROBIOLOGY-SGM 147 pp. 1765-1774, 2001, and in Burkovski, A, Kramer, R. APPLIED MICROBIOLOGY AND BIOTECHNOLOGY 58 pp. 265-274. 2002 and in references cited within.

For example, the lysine import capacity can be improved by reducing or eliminating lysine exporter activity or an enhancing lysine permease activity or an enhancing lysine/cadaverine anti-porter activity or any combination thereof.

A lysine exporter activity can be due to any polypeptide, being able to transport lysine from the medium to the cell, e.g. lysine-exporter, -symporter or -antiporter polypeptides.

In case the microorganism has an extracellular lysine decarboxylase activity and a high lysine production capacity, it will be of advantage to enhance the lysine export capacity by taking measures contrary to the ones described for enhancing the lysine import capacity, e.g. by enhancing a certain gene expression instead of reducing the expression of a certain gene. Exampies for microorganisms having a high lysine production capacity and a reduced or eliminated expression of a lysine exporter polypeptide, but lacking a extracellular lysine decarboxylase activity can be found in WO 97/23597 and WO2005073390, which disclose methods for enhanced production of amino acids by culturing microorganism having an enhanced expression or activity of amino acid export proteins and in U.S. Pat. No. 7,435,584, which discloses a method for enhanced production of L-lysine by culturing corynebacteria having a high expression of the lysE (lysine export carrier) gene.

In one embodiment of the invention the activity of at least one lysine exporter polypeptide is decreased, wherein the lysine exporter polypeptide comprises an amino acid sequence being at least 80% or at least 85% or at least 90% or at least 95% or at least 98% identical to SEQ ID NO:3 and having lysine export activity or wherein the lysine exporter polypeptide comprises an amino acid sequence being at least 80% or at least 85% or at least 90% or at least 95% or at least 98% identical to SEQ ID NO: 4 and having lysine export activity, or wherein the activity of at least two lysine exporter polypeptides are decreased, at least one comprising an amino acid sequence being at least 80% or at least 85% or at least 90% or at least 95% or at least 98% identical to SEQ ID NO: 3 and having lysine export activity and at least one comprising an amino acid sequence being at least 80% or at least 85% or at least 90% or at least 95% or at least 98% identical to SEQ ID NO: 4 and having lysine export activity.

The lysE gene has been described in the literature, Eggeling, L, Sahm, H JOURNAL OF BIOSCIENCE AND BIOENGINEERING 92, 3 201-213, Eggeling, L, Sahm, H ARCHIVES OF MICROBIOLOGY 180, 3 155-160 2003, see also the German patent application DE 95-01048222.

The YbjE gene product (SEQ ID NO: 4) and mutants thereof have been described, for example in WO2005073390.

The term “decreased activity” includes the expression of a gene product, e.g. of a lysine exporter molecule, spermidine synthase, homoserine dehydrogenase or others, at a lower level than that expressed prior to manipulation of the microorganism or in a comparable microorganism which has not been manipulated, preferably the expression of a gene product is compared to a well known strain of a particular microorganism species, which is grown under the same conditions, e.g. in the case of Corynebacterium, the expression of a gene is preferably compared with the expression level in the strain ATCC13032. in the case of E. coli, the expression of a gene is preferably compared with the expression level in the strain MG1665, deposited in the strain collection ATCC in the case of Saccharomyces cerevisiae, the expression level is compared to the strain W303 deposited in the strain collection ATCC. In one embodiment, the microorganism can be genetically manipulated (e.g., genetically engineered) to express a level of gene product at a lesser than that expressed prior to manipulation of the microorganism or in a comparable microorganism which has not been manipulated. Genetic manipulation can include, but is not limited to, altering or modifying regulatory sequences or sites associated with expression of a particular gene (e.g., by removing strong promoters, inducible promoters or multiple promoters), modifying the chromosomal location of a particular gene, altering nucleic acid sequences adjacent to a particular gene such as a ribosome binding site or transcription terminator, decreasing the copy number of a particular gene, modifying proteins (e.g., regulatory proteins, suppressors, enhancers, transcriptional activators and the like) involved in transcription of a particular gene and/or translation of a particular gene product, or any other conventional means of decreasing expression of a particular gene routine in the art (including but not limited to use of antisense nucleic acid molecules, or other methods to knock-out or block expression of the target protein). In particular the gene can be manipulated that 1 or more nucleotides are being deleted from the chromosome of the host organism. The decreased activity of the gene product e.g. of a lysine exporter molecule can also be obtained by introducing one or more gene mutations which lead to an decreased activity of the gene product. In addition the activity of a gene product can be also decreased by influencing regulatory proteins that are regulating the expression or activity of said gene product e.g. by influencing the transcription of the said gene. Examples are transcriptional repressors and transcriptional activators. For example lysE gene expression is negatively influenced by the lysG gene product. The sequence of the lysG gene (herein disclosed as SEQ ID NO: 5) and the LysG gene product (herein disclosed as SEQ ID NO: 6 can also be found under the following accession numbers: P94632, X96471

In another embodiment the lysine import capacity is enhanced by an enhanced lysine permease activity or by an enhanced lysine/cadaverine antiporter activity or a combination of both.

The lysine permease activity of a given microorganism can be enhanced for example by increasing the expression of one or more endogenous lysine permease genes for example as disclosed in SEQ ID NO: 7, or increasing the permease activity of lysine permease polypeptides for example as disclosed in SEQ ID NO: 8, e.g. by random mutagenesis of the microorganism or by transforming the microorganism with one or more lysine permease genes or by any combination thereof.

In one embodiment the lysine permease activity of the microorganism is enhanced, by recombinant expression of one or more lysine permease polypeptides comprising an amino acid sequence, which is at least 80% or at least 85% or at least 90% or at least 95% or at least 98% identical to SEQ ID NO: 8 and having lysine permeate activity, e.g. homologues or mutants having lysine permease activity. Methods to determine the lysine permease activity can be found in Bellmann, A, Vrljic, M, Patek, M, et al. MICROBIOLOGY-SGM 147 pp. 1765-1774 2001, and in Burkovski, A, Kramer, R APPLIED MICROBIOLOGY AND BIOTECHNOLOGY 58 pp. 265-274 2002, in Fujii, T, eta I. as well as in BIOSCIENCE BIOTECHNOLOGY AND BIOCHEMISTRY 66, pp 1981-1984, 2002 and in references cited therein.)

Recombinant expression can be archived for example by transformation of one or more lysine permease genes, by providing endogenous lysine permease genes with a deregulated promoter having a higher expression activity or by reducing negative regulators of lysine permease gene expression or gene product activity.

In another embodiment the lysine import capacity is enhanced by an enhanced lysine/cadaverine antiporter activity. Lysine/cadaverine antiporter are proteins transporting lysine and cadaverine at the same time in different directions across the membrane of the cell. Enhanced lysine/cadaverine antiporter activity can be archived by increasing the expression of one or more endogenous lysine/cadaverine antiporter genes, by random mutation of the microorganism, transforming the microorganism with one or more lysine/cadaverine antiporter genes or by optimizing the lysine/cadaverine antiporter activity of lysine/cadaverine antiporter polypeptides e.g. for a preference of lysine import and cadaverine export, or by any combination thereof.

In one embodiment the lysine/cadaverine antiporter activity of a microorganism is enhanced by recombinant expression of one or more lysine/cadaverine antiporter polypeptides comprising an amino acid sequence, which is at least 80% or at least 85% or at least 90% or at least 95% or at least 98% identical to SEQ ID NO: 9 having lysine/cadaverine antiporter activity, e.g. homologues or mutants having lysine/cadaverine antiporter activity.

Recombinant expression can be archived for example by transformation of one or more lysine/cadaverine antiporter genes, by providing endogenous lysine/cadaverine antiporter genes with a deregulated promoter having a higher expression activity or by reducing negative regulators of lysine/cadaverine antiporter gene expression or gene product activity.

It is known that the import or export activity of the cadB gene product, herein disclosed as SEQ ID NO: 9, depends on the lysine and cadaverine concentrations in the cell and the fermentation medium. Therefore, the lysine import capacity of a certain microorganism expressing a functional cadB gene product can be improved by enhancing the lysine concentration in the fermentation medium, preventing a low pH-value of the fermentation medium or by inserting mutations, which promote the lysine import at a given lysine concentration or pH-value in the fermentation medium or by a combination thereof (Soksawatmaekhin W. et al.; Excretion and uptake of α-daverine by CadB and its physiological functions in Escherichia coli; Molecular Microbiology; 2004; volume 51, 5; pages 1401 to 1412).

Several mutations are described, which promote the lysine import of the Escherichia coli cadB gene product a given lysine concentration or pH-value in the fermentation medium (Soksawatmaekhin W. et al.; Identification of the Cadaverine Recognition Site on the Cadaverine-Lysine Antiporter CadB; The Journal of Biological Chemistry; 2006; volume 281, 39; pages 29213 to 29220). A person having skill in the art will be able to identify similar mutations in other cadB proteins, e.g. homologues or mutants of cadB.

Accordingly, in one embodiment the lysine/cadaverine antiporter polypeptides comprise an amino acid sequence, which is at least 80% or at least 85% or at least 90% or at least 95% or at least 98% identical to SEQ ID NO: 9 comprises one or more of the following mutations: C12S, W41L, W43L, Y55L, Y57L, Y73L, Y73F, Y73W, Y89L, Y89F, Y89W, Y90L, Y90F, Y90W, Y107L, Y174L, C125S, D185N, E204Q, E204D, Y235L, Y235F, Y235W, W289L, D303N, D303E, Y310L, Y366L, Y368L, D372N, E377Q, E408Q, Y423L, Y423F and Y423W. The mutations mentioned above are named according to the amino acid sequence of the cadB gene product. A person skilled in the art will be able to transfer these mutations to other gene products, e.g. gene products being homologous of the cadB gene product, by using sequence comparison tools e.g. by using sequence alignments.

Preferably the lysine/cadaverine antiporter polypeptides comprising an amino acid sequence, which is at least 80% or at least 85% or at least 90% or at least 95% or at least 98% identical to SEQ ID NO: 9 comprises one or more of the following mutations: W41L, W43L, Y57L, Y89L, Y107L, Y174L, D185N, E204Q, Y235L, W289L, D303N, Y366L, Y368L, D372N and E408Q.

More preferred the lysine/cadaverine antiporter polypeptides comprising an amino acid sequence, which is at least 80% or at least 85% or at least 90% or at least 95% or at least 98% identical to SEQ ID NO: 9 comprises one or more of the following mutations: W41L, W43L, Y57L, Y107L, Y174L, D185N, Y366L, Y368L and E408Q.

In a further embodiment the invention provides for a microorganism comprising an intracellular lysine decarboxylase activity and an enhanced lysine import activity or comprising an intracellular and an extracellular lysine decarboxylase activity or comprising an intracellular and an extracellular lysine decarboxylase activity and an enhanced lysine import activity and having a high lysine production capacity. A microorganism having a high lysine production capacity is able to enrich lysine e.g. inside the cell or in the surrounding medium, if the lysine is not further metabolized e.g. to cadaverine.

Preferably the microorganism having a high lysine production capacity has at least one deregulated gene selected from the group (i). The group (i) is a group of genes which play a key role in the biosynthesis of lysine and consists of the genes of aspartokinase, aspartatesemialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diaminopimelate epimerase, diaminopimelate dehydrogenase, arginyl-tRNA synthetase, diaminopimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenolpyruvate carboxykinase, succinyl-CoA synthetase, methylmalonyl-CoA mutase, diamine acteyltransferase.

At least one gene of the group (i) has to be deregulated according to the inventive process. Preferably more than one gene of group (i), e.g. two, three, four, five, six, seven, eight, nine, ten genes are deregulated in the microorganism according to the invention.

Preferred genes of the group (i) to be deregulate are: aspartokinase, aspartatesemialdehyde dehydrogenase, dihydrodipicolinate synthase, dihydrodipicolinate reductase, diaminopimelate dehydrogenase, diaminopimelate decarboxylase, pyruvate carboxylase, glucose-6-phosphate dehydrogenase, transketolase, transaldolase, 6-phosphogluconolactonase, fructose 1,6-biphosphatase, homoserine dehydrogenase, succinyl-CoA synthetase, diamine actyltransferase.

The genes and gene products of group (i) are known in the art. EP 1108790 discloses mutations in the genes of homoserinedehydrogenase and pyruvate carboxylase which have a beneficial effect on the productivity of recombinant corynebacteria in the production of lysine. WO 00/63388 discloses mutations in the gene of aspartokinase which have a beneficial effect on the productivity of recombinant corynebacteria in the production of lysine. EP 1108790 and WO 00/63388 are incorporated by reference with respect to the mutations in these genes described above.

In the table below for every gene/gene product possible ways of deregulation of the respective gene are mentioned. The literature and documents cited in the row “Deregulation” of the table are herewith incorporated by reference with respect to gene deregulation. The ways mentioned in the table are preferred embodiments of a deregulation of the respective gene.

TABLE 1

| Enzyme (gene product) | Gene | Deregulation |
|---|---|---|
| Aspartokinase | ask | Releasing feedback inhibition by |
| | | point mutation (Eggeling et al., |
| | | (eds.), Handbook of Corynebacterium |
| | | glutamicum, pages 20.2.2 |
| | | (CRC press, 2005)) and amplification) |
| Aspartatesemialdehyde dehydrogenase | asd | Amplification |
| Dihydrodipicolinate synthase | dapA | Amplification |
| Dihydrodipicolinate reductase | dapB | Amplification |
| Tetrahydrodipicolinate succinylase | dapD | Amplification |
| Succinyl-amino-ketopimelate transaminase | dapC | Amplification |
| Succinyl-diamino-pimelate desuccinylase | dapE | Amplification |
| Diaminopimelate dehydrogenase | ddh | Amplification |
| Diaminopimelate epimerase | dapF | Amplification |
| Arginyl-tRNA synthetase | argS | Amplification |
| Diaminopimelate decarboxylase | lysA | Amplification |
| Pyruvate carboxylase | pycA | Releasing feedback inhibition by |
| | | point mutation (EP1108790) and |
| | | amplification |
| Phosphoenolpyruvate carboxylase | ppc | Amplification |
| Glucose-6-phosphate dehydrogenase | G6PDHzwf | Releasing feedback inhibition by |
| | | point mutation (US2003/0175911) |
| | | and amplification |
| Transketolase | tkt | Amplification |
| Transaldolase | tal | Amplification |
| 6-Phosphogluconolactonase | pgl | Amplification |
| 6-Phosphogluconate dehydrogenase | | point mutation and amplification |
| Fructose 1,6-biphosphatase | fbp | Amplification |
| Homoserine dehydrogenase | hom | Attenuating by point mutation |
| | | (EP1108790) decrease of gene |
| | | activity, Knock-out or silencing by |
| | | mutation |
| Phophoenolpyruvate carboxykinase | pck | Knock-out or silencing by mutation, |
| | | decrease of gene activity or others |
| Succinyl-CoA synthetase | sucC | Attenuating by point mutation (WO |
| | | 05/58945) decrease of gene activity |
| | | silencing by mutation |
| Methylmalonyl-CoA mutase | MMCM | Attenuating by point mutation (WO |
| | | 05/58945) decrease of gene activity, |
| | | Knock-out or silencing by mutation |
| Diamine Acetyltransferase | RXA2240 | Weakening by decrease of gene |
| | | activity, Knock-out or silencing by |
| | | mutation, deletion |

A preferred way of deregulation of the genes of aspartokinase, aspartatesemialdehyde dehydrogenase, dihydrodipicolinate synthase, dihydrodipicolinate reductase, tetrahydrodipicolinate succinylase, succinyl-amino-ketopimelate transaminase, succinyl-diamino-pimelate desuccinylase, diaminopimelate epimerase, diaminopimelate dehydrogenase, arginyl-tRNA synthetase, diaminopimelate decarboxylase, pyruvate carboxylase, phosphoenolpyruvate carboxylase, glucose-6-phosphate dehydrogenase, transketolase, transaldolase, 6-phosphogluconolactonase, fructose 1,6-biphosphatase is an “up”-mutation which increases the gene activity e.g. by gene amplification using strong expression signals and/or point mutations which enhance the enzymatic activity.

A preferred way of deregulation of the genes of homoserine dehydrogenase, phophoenolpyruvate carboxykinase, succinyl-CoA synthetase, methylmalonyl-CoA mutase Acetyltransferase is a “down”-mutation which decreases the gene activity e.g. by gene deletion or disruption, using weak expression signals and/or point mutations which destroy or decrease the enzymatic activity.

If aspartokinase is deregulated as a member of gene (i) group at least a second gene (i) member—other than aspartokinase—has to be deregulated also.

It has been observed that a significant portion of the cadaverine produced in the microorganism according to the inventive process may become acetylated later on (WO 2007/113127). In order to block the acetylation reaction which is attributed to an acetylcadaverine-forming polypeptide, which is defined as an enzymatic active polypeptide being able to produce acetylcadaverine. In order to increase the yield of cadaverine it is a preferred embodiment of the invention to deregulate the diamine acetyltransferase of the producing microorganism, especially to decrease its activity, e.g. by deletion or disruption of the gene.

One example for an acetylcadaverine-forming polypeptide is the acetyl-CoA dependent diamine acetyltransferase of Corynebacterium glutamicum (NP_{—}600742 protein) for example as disclosed in SEQ ID NO: 10 and SEQ ID NO: 11

In one embodiment of the invention, the acetylcadaverine-forming activity is decreased by decreasing the activity of at least one acetylcadaverine-forming polypeptide comprising an amino acid sequence, being at least 80%, or at least 85%, or at least 90%, or at least 95%, or at least 98% identical to SEQ ID NO: 11 and has acetylcadaverine-forming activity. Actylcadaverineforming activity can be tested as described in WO 2007/113127.

It has been observed that a significant portion of the cadaverine produced in the microorganism according to the inventive process may be converted to aminopropylcadaverine by an aminopropylcadaverine-forming polypeptide. In order to block this reaction and in order to increase the yield of cadaverine it is a preferred embodiment of the invention to deregulate the aminopropylcadaverine-forming polypeptide of the producing microorganism, especially to decrease its activity, e.g. by deletion or disruption of the gene.

One example for an aminopropylcadaverine-forming polypeptide is the spermidine synthase of Escherichia coli, as described in Soksawatmaekhin W. et al; Molecular Microbiology; (2004) Vol. 51, 5; pages 1401 to 1412))

Accordingly, in one embodiment of the invention, the aminopropylcadaverine-forming activity is decreased by decreasing the activity of at least one aminopropylcadaverine-forming polypeptide comprising an amino acid sequence, being at least 80%, or at least 85%, or at least 90%, or at least 95%, or at least 98% identical to SEQ ID NO: 12.

It has been observed that the production capacity of a microorganism for lysine can be improved by deregulating the activity of a homoserine dehydrogenase polypeptide of the cadaverine producing microorganism as described in JP2004222569, preferably by decreasing its activity via deletion or disruption of the gene coding for the homoserine dehydrogenase polypeptide.

Examples for a homoserine dehydrogenase polypeptide is the homoserine dehydrogenase of Corynebacterium glutamicum, herein disclosed as SEQ ID NO: 13, or homoserine dehydrogenases of Escherichia coli, herein disclosed as SEQ ID NO: 14 and SEQ ID NO: 15.

Accordingly, in one embodiment of the invention, the homoserine dehydrogenase activity is decreased by decreasing the activity of a homoserine dehydrogenase polypeptide comprising an amino acid sequence, being at least 80%, or at least 85%, or at least 90%, or at least 95%, or at least 98% identical to SEQ ID NO: 13, 14 or 15.

Methods for the determination of homoserine dehydrogenase activity can be found in. M. B. Jenkins, V. W. Woodward, Biochimica et Biophysica Acta, 1970, 212, 21-32.

In another embodiment of the invention the microorganism has a reduced capacity to degrade lysine other than by decarboxylation, e.g. by having a decreased lysine hydroxylase activity.

A lysine hydroxylase polypeptide is a polypeptide having lysine hydroxylase activity also described as lysine N6-hydroxylase [EC:1.14.13.59]. Tests for having lysine hydroxylase activity can be found in Meneely K M, and Lamb A L BIOCHEMISTRY 46 Pages: 11930-11937 2007 and in I J, Hsuch L C, Baldwin J E, et al. EUROPEAN JOURNAL OF BIOCHEMISTRY 268 Pages: 6625-6636.

One example is the lysine hydroxylase iucD of Escherichia coli CFT073 (SEQ ID NO: 16).

In a further embodiment of the invention, the lysine degradation activity is decreased by decreasing the activity of at least one polypeptide comprising an amino acid sequence being at least 80%, or at least 85%, or at least 90%, or at least 95%, or at least 98% identical to SEQ ID NO: 16 lysine hydroxylase, having lysine hydroxylase activity.

In a further embodiment of the invention, the microorganism has a deregulated spermidine forming or uptake activity or putrescine forming or uptake activity or a combination thereof.

A spermidine forming activity is brought about by a polypeptide being able to synthesize spermidine. One example for a spermidine forming polypeptide is the spermidine synthase of SEQ ID NO: 12.

A putrescine forming activity is brought about by a polypeptide being able to synthesize putrescine. One example for a putrescine forming polypeptide are the putrescine synthase of E. coli speE (e.g. as disclosed in SEQ ID NO: 17) or the ornithin decarboxylases of E. coli, speF (e.g. as discloseded in SEQ ID NO: 18)

A polypeptide having spermidine or putrescine uptake activity is a polypeptide being able to transport spermidine or putrescine or both into the cell. One example for an spermidine and or an putrescine uptake polypeptide is potE of E. coli (e.g. as disclosed in SEQ ID NO: 19), which functions as a putrescine/ornithine antiporter.

In one embodiment of the invention, the microorganism has a decreased spermidine forming or uptake activity or putrescine forming or uptake activity, wherein
a) the spermidine forming activity is deregulated by decreasing the activity of a spermidine forming polypeptide comprising an amino acid sequence being at least 80%, or at least 85%, or at least 90%, or at least 95%, or at least 98% identical to SEQ ID NO: 12, or,
b) the putrescine forming activity is deregulated by decreasing the activity of a putrescine forming polypeptide comprising an amino acid sequence being at least 80%, or at least 85%, or at least 90%, or at least 95%, or at least 98% identical to SEQ ID NO: 17, or
c) the putrescine forming activity is deregulated by decreasing the activity of a ornithine decarboxylase polypeptide comprising an amino acid sequence being at least 80%, or at least 85%, or at least 90%, or at least 95%, or at least 98% identical to SEQ ID NO: 18, or
d) the spermidine or putrescine or spermidine and putrescine uptake activity is deregulated by decreasing the activity of a spermidine or putrescine or spermidine and putrescine uptake polypeptide comprising an amino acid sequence being at least 80%, or at least 85%, or at least 90%, or at least 95%, or at least 98% identical to SEQ ID NO: 19, or
e) wherein the spermidine forming or uptake activity or the putrescine forming or uptake activity or a combination thereof is decreased by decreasing the activity of a combination of a), b), c) or d).

An important aspect of the present invention involves cultivating or culturing the recombinant microorganisms described herein, such that the desired compound cadaverine is produced.

Accordingly one embodiment of the invention is a cadaverine production system comprising a microorganism, comprising an intracellular lysine decarboxylase activity and an enhanced lysine import activity or comprising an intracellular and an extracellular lysine decarboxylase activity or comprising an intracellular and an extracellular lysine decarboxylase activity and an enhanced lysine import activity and a fermentation medium suitable to cultivate this microorganism, preferably the fermentation medium comprises lysine.

A cadaverine production system is a technical system for the production of cadaverine, e.g. a culture medium comprising a cadaverine producing microorganism or a lysine comprising solution or culture medium and a lysine decarboxylase producing microorganism. Usually the cadaverine production system comprises technical systems to support the production of cadaverine, e.g. a fermenter.

The term “cultivating” includes maintaining and/or growing a living microorganism of the present invention (e.g., maintaining and/or growing a culture or strain). In one embodiment, a microorganism of the invention is cultured in liquid media. In another embodiment, a microorganism of the invention is cultured on solid media or semi-solid media. In a preferred embodiment, a microorganism of the invention is cultured in media (e.g., a sterile, liquid media) comprising nutrients essential or beneficial to the maintenance and/or growth of the microorganism.

Carbon sources which may be used include sugars and carbohydrates, such as for example glucose, sucrose, lactose, fructose, maltose, molasses, starch and cellulose, oils and fats, such as for example soy oil, sunflower oil, peanut oil and coconut oil, fatty acids, such as for example palmitic acid, stearic acid and linoleic acid, alcohols, such as for example glycerol and ethanol, and organic acids, such as for example acetic acid. In one preferred embodiment, glucose, fructose or sucrose are used as carbon sources. These substances may be used individually or as a mixture.

In another preferred embodiment, the microorganisms described herein are cultivated in or on liquid, solid or semi-solid media comprising xylose, arabinose, cellobiose or mixtures thereof, such media may or may not comprise other carbon sources like the ones described above. Media for cultivating microorganisms of the invention can comprise only a limited number of different carbon sources e.g. 1, 2, 3, 4, 5 or more carbon sources, or might comprise very complex mixtures of carbon sources, hydrolysates of lignocellulose substrates or argricultural residues, e.g. hydrolysates of starches from sources such as but not limited to corn, wheat, rye, barley, rice, cassaya, or hydrolysates of straw, wood, paper, or other material of plant origin.

Preferred combinations of carbon sources are media comprising a high content of glucose and xylose, fructose and xylose, sucrose and xylose, or sucrose and glucose, or sucrose and fructose, or sucrose, glucose and fructose and sucrose, glucose, fructose and xylose, or glucose, fructose and xylose or glucose, fructose, xylose and arabinose or sucrose, xylose and arabinose, or glucose, xylose and arabinose and of other combination of sugars mentioned.

In case the media comprises xylose it is of advantage to use a microorganism of the invention expressing or overexpressing genes of the xylose metabolism.

Genes of the xylose metabolism are for example the genes of the xyIABFGHR locus of E. coli, comprising genes for a xylose transport systems (xylE, xylT and the xyIFGH gene), genes for xylose utilization (xylA and xylB gene) and genes for xylose transcriptional activator (xylR gene). Microorganisms overexpressing genes of the xyIABFGHR locus are described in EP1577396 and EP1577369. Corynebacteria overexpressing genes of the xylA alone or with the xylB gene of E. coli, encoding a xylose isomerase and xylB encoding a xylulokinase have been described e.g. (Kawaguchi, et al. Engineering of a xylose metabolic pathway in Corynebacterium gutamicum, Applied and Environmental Microbiology, 2006, Vol. 72, 5, pages 3418 to 3428).

In a preferred embodiment, the microorganism of the invention expresses or over expresses at least the xylA or the xylB gene or even more preferred the xylA and the xylB gene.

In case the media comprises arabinose, it is of advantage to use a microorganism of the invention expressing or overexpressing genes of the arabinose metabolism. Genes of the arabinose metabolism are for example genes of the araBAD operons, e.g. the araA, araB, araD and areE genes of E. coli, coding for L-arabinose isomerase (araA), L-ribolokinase (araB) and L-ribulose-5-phosphate-4-epimerase (araD) or the genes of the araBDA operon of Corynebacterium gluctamicum, comprising homologs of the araA, araB and araD genes and the araE coding for a L-arabinose isomerase, the araR gene coding for a transcriptional regulator and the galM gene coding for a putative aldose 1-epimerase. Preferably the microorganism of the invention expresses or overexpresses at least the araA, araB and araD gene, more preferably at least the araA, araB, araD and the araE gene. Kawaguchi et al. Identification and Functional Analysis of the Gene Cluster for L-Arabinose Utilization in Corynebacterium glutamicum, Applied and Environmental Microbiology, 2009, 75, Vol. 11, pages 3419-3429).

The E. coli homologs of the genes of the arabinose metabolism can also be used in heterologous microorganism such as Corynebacterium glutamicum (Kawaguchi et al. Engineering of an L-arabinose metabolic pathway in Corynebacterium glutamicum, Applied Microbiology and Biotechnology, 2008, 77, Vol, 5, pages 1053 to 1062).

In case the media comprises cellobiose, it is of advantage to use a microorganism of the invention expressing or overexpressing genes of the cellobiose metabolism. Genes of the cellobiose metabolism are for example the bglA genes of Corynebacterium glutamicum, encoding phosphenolpyruvate:carbohydratephosphotransferase system (PTS) beta-glucoside-specific enzyme IIBCA component and phosphor-beta-glucosidase examples of these genes and the respective proteins from the Corynebacterium R strain can be found under the accession number AF508972.

In case the media comprises combinations of xylose, arabinose, cellobiose or other carbon sources, it is of advantage to use microorganisms of the invention expressing or overexpressing genes of the xylose metabolism, arabinose metabolism or cellobiose metabolism. For example, in case the media has a high content of xylose- and arabinose it is of advantage to use microorganisms expressing or overexpressing genes of the xylose- and arabinose metabolism for example expressing the xylA and xylB genes and the araA, araB, araD genes, preferably the xylA and xylB araA, araB, araD and araE gene.

In case the media has a high content xylose and cellubiose it is of advantage to use microorganisms expressing or overexpressing genes of the xylose- and cellobiose metabolism for example expressing the xylA and xylB and the bglA genes (Sasaki et al. Simultaneous utilization of D-cellobiose, D-glucose, and D-xylose by recombinant Corynebacterium glutamicum under oxygen-deprived conditions, Applied Microbiology and Biotechnology, 2008, Vol. 81, 4, pages 691 to 699)

Nitrogen sources which may be used comprise organic compounds containing nitrogen, such as peptones, yeast extract, meat extract, malt extract, corn steep liquor, soya flour and urea or inorganic compounds, such as ammonium sulfate, ammonium chloride, ammonium phosphate, ammonium carbonate and ammonium nitrate. The nitrogen sources may be used individually or as a mixture. Phosphorus sources which may be used are phosphoric acid, potassium dihydrogen phosphate or dipotassium hydrogen phosphate or the corresponding salts containing sodium. The culture medium must furthermore contain metal salts, such as for example magnesium sulfate or iron sulfate, which are necessary for growth. Finally, essential growth-promoting substances such as amino acids and vitamins may also be used in addition to the above-stated substances. Suitable precursors may furthermore be added to the culture medium. The stated feed substances may be added to the culture as a single batch or be fed appropriately during cultivation.

Preferably, microorganisms of the present invention are cultured under controlled pH. The term “controlled pH” includes any pH which results in production of the desired fine chemical, e.g. cadaverine. In one embodiment, microorganisms are cultured at a pH of about 7. In another embodiment, microorganisms are cultured at a pH of between 6.0 and 8.5. The desired pH may be maintained by any number of methods known to those skilled in the art. For example, basic compounds such as sodium hydroxide, potassium hydroxide, ammonia, or ammonia water, or acidic compounds, such as phosphoric acid or sulfuric acid, are used to appropriately control the pH of the culture.

Also preferably, microorganisms of the present invention are cultured under controlled aeration. The term “controlled aeration” includes sufficient aeration (e.g., oxygen) to result in production of the desired fine chemical, e.g., cadaverine. In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the growth vessel (e.g., fermentor) or by various pumping equipment. Aeration may be further controlled by the passage of sterile air or oxygen through the medium (e.g., through the fermentation mixture). Also preferably, microorganisms of the present invention are cultured without excess foaming (e.g., via addition of antifoaming agents such as fatty acid polyglycol esters).

Moreover, microorganisms of the present invention can be cultured under controlled temperatures. The term “controlled temperature” includes any temperature which results in production of the desired fine chemical, e.g., cadaverine. In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 45° C. or between 30° C. and 50° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g., a fermentation process). Fermentation processes of the present invention include, but are not limited to, batch, fed-batch and continuous methods of fermentation. The phrase “batch process” or “batch fermentation” refers to a closed system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to a system in which a defined fermentation medium is added continuously to a fermentor and an equal amount of used or “conditioned” medium is simultaneously removed, preferably for recovery of the desired cadaverine. A variety of such processes have been developed and are well-known in the art.

The methodology of the present invention can further include a step of recovering cadaverine. The term “recovering” cadaverine includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like. For example cadaverine can be recovered from culture media by first removing the microorganisms. The broth removed biomass is then passed through or over a cation exchange resin to remove unwanted cations and then through or over an anion exchange resin to remove unwanted inorganic anions and organic acids having stronger acidities than cadaverine. In addition the broth can be treated with caustic agents and the cadaverine be extracted with organic solvents such as alkohols by phase separation. The cadaverine can be retrieved from the extracted phase by distillation to purity sufficient for diverse applications. Possible applications include the production of polyamides by polycondensation with dicarboxylic organic acids.

Accordingly, in another aspect, the present invention provides a process for the production of polyamides (e.g. Nylon®) comprising a step as mentioned above for the production of cadaverine. The cadaverine is reacted in a known manner with di-, tri- or polycarboxylic acids to get polyamides. Preferably the cadaverine is reacted with dicarboxylic acids containing 4 to 10 carbons such as succinic acid, glutaric acid, adipic acid, pimelic acid, suberic acid, azelaic acid, sebacic acid, and so forth. The dicarboxylic acid is preferably a free acid.

The microorganism of the invention are useful for producing cadaverine by fermenting these microorganism, which is growing the microorganism in culture, preferably growing the microorganism under culture conditions as described above.

Accordingly the invention includes a process for the production of cadaverine, comprising fermenting a microorganism, comprising an intracellular lysine decarboxylase activity and an enhanced lysine import activity or comprising an intracellular and an extracellular lysine decarboxylase activity or comprising an intracellular and an extracellular lysine decarboxylase activity and an enhanced lysine import activity. Preferably the process includes recovering of cadaverine from the culture medium.

In one embodiment the microorganism comprises an enhanced lysine/cadaverine antiporter activity, e.g. a microorganism overexpressing a polypeptide which is at least 80% or at least 85% or at least 90% or at least 95% or at least 98% identical to SEQ ID NO: 9 and having lysine/cadaverine antiporter activity, e.g. homologues or mutants having lysine/cadaverine anti-porter activity and the process includes fermenting the microorganism in a medium comprising lysine. Preferably the culture medium comprises more than 0.1 mM, or more than 0.5 mM, or more than 1 mM, or more than 3 mM, or more than 5 mM, or more than 7 mM, or more than 8 mM, or more than 9 mM, or more than 10 mM lysine. More preferred the culture medium comprises more than 15 mM lysine. Even more preferred, the culture medium comprises more than 20 mM lysine. Most preferred the culture medium comprises more than 30 mM lysine

In a further embodiment the microorganism comprises an extracellular lysine decarboxylase activity or comprises an extracellular lysine decarboxylase activity and an enhanced lysine/cadaverine antiporter activity.

In another embodiment of the invention is a process to produce cadaverine, wherein the concentration (mol/l) of cadaverine in the culture medium is at least 1.2 times higher, or more than 1.3 times higher, or more than 1.4 times, or more than 1.5 times, or more than 1.6 times, or more than 1.7 times, or more than 1.8 times, or more than 1.9 times or more than 2 times, 3 times, 4 times, 5 times, 6 times, 7 times, 8 times, 9 times higher than the concentration (mol/l) of acetylcadaverine or aminopropylcadaverine or of both. Another embodiment of the invention is the culture medium produced in the process.

Preferably the culture medium comprises cadaverine and a very low concentration of acetylcadaverine or aminopropylcadaverine. More preferably, the concentration of acetylcadaverine or aminopropylcadaverine or both is lower than 0.2, or 0.1, or 0.05, or 0.03, or 0.01 mol/l, or 0.005 mol/l or 0.001 mol/l. Preferably the concentrations of cadaverine, acetylcadaverine or aminopropylcaderine are determined after the process for production has been stopped, e.g. after the microorganisms producing the cadaverine have been separated from the culture medium.

A further embodiment of the invention is a process to produce cadaverine, wherein the concentration (mol/l) of cadaverine in the culture medium is at least 1.2 times higher, or more than 1.3 times higher, or more than 1.4 times, or more than 1.5 times, or more than 1.6 times, or more than 1.7 times, or more than 1.8 times, or more than 1.9 times or more than 2 times, 3 times, 4 times, 5 times, 6 times, 7 times, 8 times, 9 times, 10 times, 11 times higher than the concentration (mol/l) of acetylcadaverine. Another embodiment is the culture medium produced in the process.

In further embodiment of the invention is a process to produce cadaverine, wherein the concentration (mol/l) of cadaverine in the culture medium is at least 1.2 times higher, or more than 1.3 times higher, or more than 1.4 times, or more than 1.5 times, or more than 1.6 times, or more than 1.7 times, or more than 1.8 times, or more than 1.9 times or more than 2 times, 3 times, 4 times, 5 times, 6 times, 7 times, 8 times, 9 times, 10 times, 11 times higher than the concentration (mol/l) of aminopropylcadaverine. Another embodiment is the culture medium produced in the process.

Another embodiment of the invention is a culture medium comprising at least 0.25 mol/l, or at least 0.3, or at least 0.35, or at least 0.4, or at least 0.45, or at least 0.5, or at least 0.6, or at least 0.7, or at least 0.8, or at least 0.9 mol/l cadaverine and not more than 0.2 mol/l, or not more than 0.15 mol/l, or not more than 0.1 mol/l, or not more than 0.05, or not more than 0.045, or not more than 0.04 or not more than 0.03 mol/l acetylcadaverine, or not more than 0.05, or not more than 0.045, or not more than 0.04 or not more than 0.03 mol/l aminopropylcadaverine, or not more than 0.05, or not more than 0.045, or not more than 0.04 or not more than 0.03 mol/l of each acetylcadaverine and aminopropylcadaverine.

The cadaverine produced by the processes described above can be recovered or purified by a work up of the cadaverine (DAP) comprising fermentation broth, i.e. the culture medium in its state after the process to produce cadaverine is finished or has been terminated.

The process to recover or purify the cadaverine from the fermentation broth as described in the following medium is solely for exemplary reasons. The person skilled in the art will know alternative methods or variants of the process described below, which can also successfully be applied.

In order to recover the produced cadaverine, it is of advantage to thicken or to concentrate the fermentation broth. The fermentation broth can be thickened or concentrated by known methods, such as, for example, with the aid of a rotary evaporator, thin-film evaporator, falling film evaporator, by reverse osmosis or by nanofiltration. If necessary, salts which may have precipitated due to the concentration procedure may be removed, for example by filtration or centrifugation. This concentrated fermentation broth can then be worked up in the manner of the invention to obtain cadaverine. For the work up in accordance with the present invention, such a concentration procedure is feasible, but not absolutely necessary.

According to the invention, cadaverine is extracted from the fermentation broth with the aid of an organic extractant. More specifically, use is made of here of an organic solvent having a miscibility gap with water that is as polar as possible and stable at alkaline pH, such as in particular a polar, dipolar protic, organic solvent. Suitable solvents are in particular cyclic or openchain, optionally branched alkanols having from 3 to 8 carbon atoms, in particular n- and isopropanol, n-, sec- and iso-butanol, or cyclohexanol, and also n-pentanol, n-hexanol- n-heptanol, n-octanol, 2-octanol and the mono- or polybranched isomeric forms thereof. Particular mention is to be made here of n-butanol.

In a preferred embodiment, the extraction and/or subsequent phase separation are carried out batchwise at an elevated temperature which is limited by the boiling points of water and of the extractant or of possibly forming azeotropes. Using the extractant n-butanol, extraction and phase separation could be carried out, for example, at about 25-90° C. or, preferably, at 40-70° C. For extraction, the two phases are stirred until the partition equilibrium has been established, for example over a period of from 10 seconds to 2 hours, preferably 5 to 15 min. The phases are then left to settle until they have separated completely; this takes preferably from 10 seconds to 5 hours, for example 15 to 120 or 30 to 90 minutes, in particular also at a temperature in the range from about 25-90° C. or 40-70° C. in the case of n-butanol.

In further preferred embodiments, cadaverine is extracted from the fermentation broth continuously in a multi-stage process (for example in mixer-settler combinations) or continuously in an extraction column.

The skilled working may establish the configuration of the extraction columns which can be employed according to the invention for the phases to be separated in each case as part of optimization routines. Suitable extraction columns are in principle those without power input or those with power input, for example pulsed columns or columns with rotating internals. The skilled worker may also, as part of routine work, select in a suitable manner types and materials of internals, such as sieve trays, and column trays, to optimize phase separation. The basic theories of liquid-liquid extraction of small molecules are well known (cf. e.g. H.-J. Rehm and G. Reed, Eds., (1993), Biotechology, Volume 3 Bioprocessing, Chapter 21, VCH, Weinheim). The configuration of industrially applicable extraction columns is described, for example, in Lo et al., Eds., (1983) Handbook of Solvent Extraction, JohnWiley& Sons, New York. Explicit reference is made to the disclosure of the textbooks above.

After phase separation, cadaverine is isolated and purified from the cadaverine-comprising extract phase in a manner known per se. Possible measures of recovering cadaverine are in particular, without being limited thereto, distillation, precipitation as salt with suitable organic or inorganic acids, or combinations of such suitable measures.

Distillation may be carried out continuously or batchwise. A single distillation column or a plurality of distillation columns coupled to one another may be used. Configuring the distillation column apparatus and establishing the operational parameters are the responsibilities of the skilled worker. The distillation columns used in each case may be designed in a manner known per se (see e.g. Sattler, Thermische Trennverfahren [Thermal separation methods], 2nd Edition 1995, Weinheim, p. 135ff; Perry's Chemical Engineers Handbook, 7th Edition 1997, New York, Section 13). Thus, the distillation columns used may comprise separation-effective internals, such as separation trays, e.g. perforated trays, bubble-cap trays or valve trays, arranged packings, e.g. sheet-metal or fabric packings, or random beds of packings. The number of plates required in the column(s) used and the reflux ratio are essentially governed by the purity requirements and the relative boiling position of the liquids to be separated, with the skilled worker being able to ascertain the specific design and operating data by known methods.

Precipitation as salt may be achieved by adding suitable organic or inorganic acids, for example sulfuric acid, hydrochloric acid, phosphoric acid, acetic acid, formic acid, carbonic acid, oxalic acid, etc. In another preferred embodiment, an organic dicarboxylic acid is used, forming a salt which can be used, either directly or after purification, for example by recrystallization, in a subsequent polycondensation to give the polyamide. More specifically, such dicarboxylic acids are C₄-C₁₂-dicarboxylic acids.

The organic cadaverine phase produced in the extraction procedure may also be worked up chromatographically. For chromatography, the cadaverine phase is applied to a suitable resin, for example a strongly or weakly acidic ion exchanger (such as Lewatit 1468 S, Dowex Marathon C, Amberlyst 119 Wet or others), with the desired product or the contaminants being partially or fully retained on the chromatographic resin. These chromatographic steps may be repeated, if necessary, using the same or other chromatographic resins. The skilled worker is familiar with selecting the appropriate chromatographic resins and their most effective application. The purified product may be concentrated by filtration or ultrafiltration and stored at an appropriate temperature.

The identity and purity of the compound(s) isolated may be determined by prior art technologies. These comprise high performance liquid chromatography (HPLC), gas chromatography (GC), spectroscopic methods, staining methods, thin layer chromatography, NIRS, enzyme assay or microbiological assays. These analytical methods are summarized in: Patek et al. (1994) Appl. Environ. Microbiol. 60:133-140; Malakhova et al. (1996) Biotekhnologiya 11 27-32; and Schmidt et al. (1998) Bioprocess Engineer. 19:67-70. Ullmann's Encyclopedia of Industrial Chemistry (1996) Vol. A27, VCH: Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

As the cadaverine produced and recovered or purified by the processes described above can be used to produce polyamides by known techniques, those polyamides represent another embodiment of the invention.

The invention will now be described in more detail on the basis of the following non-limiting examples and with reference to the accompanying figures.

### Example 1

### Strain Construction of LU11697

C. glutamicum strain ATCC 13032 was transformed with DNA A (also referred to as pint sacB delta PEPCK) (SEQ ID NO:20) and “Campbelled in” to yield a “Campbell in” strain. The “Campbell in” strain was then “Campbelled out” to yield a “Campbell out” strain, LU11031, which contains a modified phosphoenolpyuvat carboxykinase gene (pepck) encoding a defective phosphoenolpyuvat carboxykinase enzyme (Delta PEPCK).

The strain 11301 was subsequently transformed with DNA B (also referred to as pint sacB 2× ddh) (SEQ ID NO:21) to yield a “Campbell in” strain. The “Campbell in” strain were then “Campbelled out” to yield a “Campbell out” strain, LU11100, which contains a gene encoding a duplicated ddh gene with its native promotors (2*ddh).

The strain LU11100 was transformed with DNA C (also referred to as pint sacB LysC T311I) (SEQ ID NO:22) to yield a “Campbell in” strain, which was then “Campbelled out” to yield a “Campbell out” strain, LU11271. The LU11271 strain contained which contains a gene encoding a feedback resistant aspartate kinase enzyme (Askfi) (encoded by IysC). In the resulting aspartate kinase protein, T311 was changed to I131 (referred to as LysC T311I). In addition a PSOD promoter is added to drive expression of the ask lysC gene.

The strain LU11271 was transformed with DNA D (also referred to as pK19 PSOD dapB rxb2957) (SEQ ID NO:23) to yield a “Campbell in” strain, which was then “Campbelled out” to yield a “Campbell out” strain, LU11269. The LU11269 strain contained a PSOD promoter upstream of the dapB gene to drive the expression of dapB.

The strain LU11269 was transformed with DNA E (also referred to as pK19 sacB 2× argS lysA) (SEQ ID NO:24) to yield a “Campbell in” strain, which was then “Campbelled out” to yield a “Campbell out” strain, LU11275. The LU11275 strain contained which contains a duplicated genetic locus containing the argS and the lysA gene.

The strain LU11275 was transformed with DNA F (also referred to as pint sacB delta hom) (SEQ ID NO:25) to yield a “Campbell in” strain, which was then “Campbelled out” to yield a “Campbell out” strain, LU11328. The LU11368 strain contains a deletion of the hsdh/hom gene.

The strain LU11368 was transformed with DNA G (also referred to as pK19 delta pycA,) (SEQ ID NO:26) to yield a “Campbell in” strain, which was then “Campbelled out” to yield a “Campbell out” strain, LU11374. The strain LU11374 contains a deleted pyruvate carboxylase gene pycA gene.

The strain LU11374 was transformed with DNA H (also referred to as pCLIk Int sacB hsdh replcaement) (SEQ ID NO:27) to yield a “Campbell in” strain, which was then “Campbelled out” to yield a “Campbell out” strain, LU11401. The strain contains mutated hom gene with the mutation V59 to A.

The strain LU11401 was transformed with DNA I (also referred to as pint sacB pycA*) (SEQ ID NO:28) to yield a “Campbell in” strain, which was then “Campbelled out” to yield a “Campbell out” strain, LU11414. The strain contains a mutated pyruvate carboxylase gene with the mutation P458 to S.

The strain LU11414 was transformed with DNA J (also referred to as pint sacB PSOD pycA) (SEQ ID NO:28) to yield a “Campbell in” strain, which was then “Campbelled out” to yield a “Campbell out” strain, 11424. The strain contains a PSOD promotor upstream of the pycA gene.

The strain LU11424 was transformed with DNA K (also referred to as pint sacB delta G6PDH) (SEQ ID NO:28) to yield a “Campbell in” strain, which was then “Campbelled out” to yield a “Campbell out” strain, 11436. containing a deletion of the glucose-6-P-dehydrogenase gene (G6PDH)

The strain LU11436 was transformed with DNA L (also referred to as pint sacB G6PDH*) (SEQ ID NO:29) to yield a “Campbell in” strain, which was then “Campbelled out” to yield a “Campbell out” strain, LU11443. containing a mutated glucose-6-P-dehydrogenase gene (G6PDH). In this gene the sequence was mutated that the aminoacid 234 was mutated to a threonine while the wildtype protein carried an alanine.

The strain LU11443 was transformed with DNA M (also referred to as pK19 sacB delta succinyl-CoA-synthetase)) (SEQ ID NO:30) to yield a “Campbell in” strain, which was then “Campbelled out” to yield a “Campbell out” strain, LU11600. containing a deleted succinyl-CoA-synthase beta chain.

The strain LU11600 was transformed with DNA N (also referred to as pK19 sacB succinyl-CoAsynthetase replacement) (SEQ ID NO:31) to yield a “Campbell in” strain, which was then “Campbelled out” to yield a “Campbell out” strain, LU11601. containing a mutated succinyl CoA synthase beta chain gene. In this gene the sequence was mutated that the aminoacid was mutated while the wildtype protein carried the following mutations P17 to S, G39 to E, A165 to T.

The strain LU11601 was transformed with DNA 0 (also referred to as pCLIK Int sacB delta MMCM) (SEQ ID NO:32) to yield a “Campbell in” strain, which was then “Campbelled out” to yield a “Campbell out” strain, LU11606, containing a deleted methylmalonyl CoA Mutase gene.

The strain LU11606 was transformed with DNA P (also referred to as pCLIK Int sacB MMCM replacement) (SEQ ID NO:33) to yield a “Campbell in” strain, which was then “Campbelled out” to yield a “Campbell out” strain, LU11696. containing a mutated methylmalonyl CoA Mutase gene the following mutations P75 to S, G413 to E, A417 to V.

The strain LU11696 was transformed with DNA Q (also referred to as pint sacB PSOD G6PDH) (SEQ ID NO:34) to yield a “Campbell in” strain, which was then “Campbelled out” to yield a “Campbell out” strain, LU11697, containing a PSOD promoter in front of the G6PDH gene.

### Example 2

### Strain Construction of LU14105

The strain LU11697 was transformed with DNA R (also referred to as pint sacB Idc bioD) (SEQ ID NO:35) to yield a “Campbell in” strain, which was then “Campbelled out” to yield a “Campbell out” strain, LU14105, containing a Lysine decarboxylase gene derived from E. coli under the transcriptional control of its native promotor (Idc) integrated into the bioD locus of C. glutamicum.

### Example 3

### Strain Construction of LU14635

The strain LU14105 was transformed with DNA S (also referred to as pint sacB delta RXA02214) (SEQ ID NO:36) to yield a “Campbell in” strain, which was then “Campbelled out” to yield a “Campbell out” strain, LU14635 containing a deletion of the acetyltransferase gene.

### Example 4

### Strain Construction of LU14646 and LU14646 Delta Acetyltransferase

The strain LU 14105 was transformed with DNA T (also referred to as pint sacB delta lysE) (SEQ ID NO:37) to yield a “Campbell in” strain, which was then “Campbelled out” to yield a “Campbell out” strain, LU14646 containing a deletion of the lysine exporter lysE gene.

The strain LU14646 was transformed with DNA R (also referred to as pint sacB delta RXA02214) (SEQ ID NO:38) to yield a “Campbell in” strain, which was then “Campbelled out” to yield a “Campbell out” strain, LU14646 delta acetyltransferase, containing a deletion of the identified acetyltransferase gene.

### Cadaverine Production in Shaking Flask Culture

Shaking flask experiments were performed on the recombinant strains to test the cadaverine production. The same culture medium and conditions as lysine production were employed as described in WO2005059139. For the control host strain and recombinant strain having pClik5aMCS were tested in parallel. The strains were pre-cultured on CM agar overnight at 30° C. Cultured cells were harvested in a microtube containing 1.5 ml of 0.9% NaCl and cell density was determined by the absorbance at 610 nm following vortex. For the main culture suspended cells were inoculated to reach 1.5 of initial OD into 10 ml of the production medium contained in an autoclaved 100 ml of Erlenmeyer flask having 0.5 g of CaCO₃. Main culture was performed on a rotary shaker (Infors AJ118, Bottmingen, Switzerland) with 200 rpm for 48-78 hours at 30° C. The concentrations of cadaverine and lysine were determined using HPLC (Agilent 1100 Series LC system).

In the broth cultured with all recombinant strains containing the lysine decarboxylase genes a significant amount of cadaverine was accumulated. On the contrary, marked decrease in the lysine productivity was observed. Considering complete conversion of lysine to cadaverine the same number of cadaverine molecules as lysine must be produced. However, the amount of cadaverine accumulated was less than that of lysine produced by host strain, on the other hand, a considerable amount of byproduct, acetylcadaverine, was concomitantly accumulated resulting in sugar yield decrease.

In addition to HPLC analysis, cadaverine and acetylcadaverine were identified by mass spectrometrical method.

### Plasmid Construction and Disruption of the Lyse Gene

For the chromosomal disruption of the gene encoding the lysine exporter a recombinant plasmid was constructed which allows the marker-free manipulation by two consecutive homologous recombination events. The DNA fragments containing the regions of the up- and the downstream of the lysE gene were amplified from wildtype ATCC13032 C. glutamicum chromosomal DNA using a sets of PCR primers (WJK199, WJK200, WJK201 and WJK202), for the upstream and for the downstream, and used as a template for fusion PCR with PCR primers to produce a fused fragment wherein the middle region of the gene is removed. The product of the fusion PCR of the size of 1039 basepairs was purified, digested with SpeI and XbaI, and inserted into the pint sacB vector, which makes the integration of sequences at the genomic locus of C. glutamicum (Becker et al (2005), Applied and Environmental Microbiology, 71 (12), p. 8587-8596), digested with same restriction enzymes.

Oligonucleotides for the amplification of lysE

WJK199
5′-TCTCACTAGTGCAGCAAGGATAATGTGTGC-3′
WJK200
5′-GTTGTCTAGAGCTGCCAACAATGGTCTTGG-3′
WJK201
5′-CACGACGACGTTGATCCAGCGGTCCGATGGACAGTAAAAG-3′
WJK 202
5′-CTTTTACTGTCCATCGGACCGCTGGATCAACGTCGTCGTG-3′

The plasmid was then used to disrupt the native coding region of the lysE gene. The strain used was LU14105 in which the IdcC gene was integrated into the bioD locus of the chromosome, which produces cadaverine and lysine. Two consecutive recombination events, one in each of the up- and the downstream region, respectively, are necessary to disrupt the middle sequence of the gene. The disrupted mutants were confirmed by PCR with primers and Southern hybridization analysis.

### Example 5

### Cadaverine Production in Shaking Flask Culture

Cadaverine production in shaking flask culture of the aforementioned strains.

The strains LU1105, LU14635, LU14646 and LU14646 delta acetyltransferase were analyzed for cadaverine production. Shaking flask experiments were performed on the afore mentioned recombinant strains carrying deletions of the lysE gene and the acetyltransferase gene to test the cadaverine production. The same culture medium and conditions as lysine production were employed as described in WO2005059139. For the control host strain and recombinant strain carrying the deleted/disrupted lysE gene were tested in parallel. The strains were pre-cultured on CM agar overnight at 30° C. Cultured cells were harvested in a microtubes containing 1.5 ml of 0.9% NaCl and cell density was determined by the absorbance at 610 nm following vortex. For the main culture suspended cells were inoculated to reach 1.5 of initial OD into 10 ml of the production medium contained in an autoclaved 100 ml of Erlenmeyer flask having 0.5 g of CaCO3. Main culture was performed on a rotary shaker (Infors AJ118, Bottmingen, Switzerland) with 200 rpm for 48-78 hours at 30° C. The concentrations of cadaverine and lysine were determined using HPLC (Agilent 1100 Series LC system).

It was found that in the broth cultured with the recombinant strain LU14646 carrying a deleted/disrupted lysE gene a the amount of cadaverine was increased from 6.6 g/l to 9.9 g/l over the isogenic strain LU14105 carrying a wildtype lysE gene. Additionally the lysine productivity was decreased from 8.3 g/ to 0.3 g/l. The deletion of the acetyltransferase also increased the cadaverine production from 6.6 g/l to 10.2 g/l while 11.8 g/l lysine accumulated in the supernatant. When the aforementioned acetyltransferase was deleted in the strain L14635 and LU14646 delta acetyltransferase no acetyl-cadaverine could be detected. In the combination of deletion of the lysE gene and the acetyltransferase, the cadaverine concentration was increased to 14.9 g/l showing a synergy between the deletion of the lysE gene and the deletion of the acetyltransferase gene.

TABLE 2

| | Product g/l | | |
|---|---|---|---|
| | cadaverine | lysine HCl | acetyl-cadaverine |
| LU14105 | 6.6 | 8.5 | 1.2 |
| LU14646 delta lysE | 9.9 | 0.3 | 0.6 |
| LU14635 (delta | 10.2 | 11.8 | 0 |
| acetyltransferase) | | | |
| LU14646 delta | 14.9 | 0.2 | 0 |
| acetyltransferase | | | |

In another embodiment a lysine decarboxylase is cloned in a fusion with a strong promoter active in Corynebacterium glutamicu and a sequence constituting a translocation and secretion sequence active in C. glutamicum. Sequences for translocation and secretion can be found identifying secreted proteins in Actinobacteria such as Streptomyces and Corynebacteria by gel electrophoresis separation and N-terminal sequencing by Edman degradation. The DNA sequence coding for secreted proteins will show an 5′ extension with 1-100 by additional sequence in front of the first codon coding for the first amino acid found in an secreted protein. This additional sequence after a start codon will code for a peptide signal sequence that targets the protein for translocation and secretion. This signal sequence can be fused in front of a gene such as lysine decarboxylase IdC or cadA to be expressed into mRNA to be produced to a fusion protein of signal sequence and protein sequence. The fusion protein will be translocated and secreted into the cell wall matrix and into the cell culture supernatant upon which the signal sequence is cleaved off and the protein is being secreted. The secreted protein, an active lysine decarboxylase such as LDC and CADA, can be active in the cell supernatant, where it is able to convert lysine into cadaverine by a decarboxylation reaction. By this, the amount of produced lysine by the cellular metabolism can be converted into cadaverine and the carbon source can be converted into cadaverine without concomitant production of side products. Most likely the expression of a secreted lysine decarboxylase is being performed in a strain that is also expressing a intracellular lysine decarboxylase as well as in a strain with a deletion in a cadaverine acetyltransferase.

### Example 6

### Strain Construction of Lysine Decarboxylase Secreting Organism

For the construction of a strain capable of secreting a functional lysine decarboxylase, the PSOD promotor as described above, the signal sequence of the alpha-amylase from Streptomyces coelicolor and the LDC gene are fused by PCR primer extension and cloned to yield the vector pCLIK5A PSOD Idc sigseq alpha amylase SEQ ID NO. 41. The resulting vector is transformed into the strain LU14635. After growth and incubation in shake flask as described above it is found that the amount of lysine in the culture supernatant is decreased compared to the control expressing IdC without translocation/secretion signal sequence.

### Example 7

### Strain Construction of for the Utilization of C5 Sugars for the Production of DAP

The E. coli genes for the utilization of xylose (the xylose isomerase and the xylulose kinase xyIAB) were fused to the Pgro promoter from C. glutamicum by the PCR fusion method, amplified by PCR and inserted into the vector pG into the restriction site XhoI and MluI to yield the vector pG Pgro xylAB SEQ ID NO. 39. The plasmid was transformed into the strain LU14635 as well as the empty control plasmid pG, which did not contain the xylose utilisation genes yielding the strains LU14635 pG and the strain LU14635 pG Pgro xylAB.

### Cadaverine Production in Shaking Flask Culture

Shake flask experiments were conducted as follows:

Cadaverine production in shaking flask culture of the aforementioned strains. The strain LU14635 pG and pG Pgro Xyl AB were analyzed for cadaverine production. Shaking flask experiments were performed with the recombinant strains with the plasmid to overexpress the xylose operon genes to test the cadaverine production. The strains were pre-cultured on CM agar with kanamycin 25 μg/ml overnight at 30° C. Cultured cells were harvested in a microtube containing 1.5 ml of 0.9% NaCl and cell density was determined by the absorbance at 610 nm following vortex. For the main culture suspended cells were inoculated to reach 1.5 of initial OD into 10 ml of the MDAP medium with added Kanamycin at a concentration of 25 μg/ml contained in an autoclaved 100 ml of Erlenmeyer flask. Main culture was performed on a rotary shaker (Infors AJ118, Bottmingen, Switzerland) with 200 rpm for 48-78 hours at 30° C. The concentrations of cadaverine and lysine and residual sugars were determined using HPLC (Agilent 1100 Series LC system).

### MDAP Batch-Medium Recipe

The MDAP Medium consists of the following components

ammoniumsulfate 25 g/L
yeast extract Difco 15 g/L
vitamine stock 15 ml/L
salt solution MDAP 100 ml/L
trace element solution: 15 ml/L
pyridoxine 1 mg/ml 1 ml/L
sugar (glucose or xylose) 60-50 g/L

### ACES 78 g/L

### MOPS 21 g/L

### CaCO3 15 g/L

### Vitamin Stock

Biotin 300 mg/l
Thiamin 500 mg/l
Nicotinamide 600 mg/l
Pantothenic acid 2000 mg/l
Addition of vitamin solution 15 ml/l

### Salt Solution MDAP

citric acid 2 g/L
calciumsulfate 1.5 g/L
potassium phosphate 25 g/L
magnesiumsulfate 12.5 g/L
iron (II) sulfate 700 mg/L
zink sulfate 300 mg/L
manganese sulfate 150 mg/L

### Trace Element Solution

citric acid 2.1 g/L
boric acid 300 mg/L
cobaltsulfate 240 mg/L
coppersulfate 300 mg/L
nickelsulfate 200 mg/L
sodium molibdate 50 mg/L

### Results:

TABLE 3
Growth and production of cadaverine by the strain
LU 14635 without and with over-expression of the
xylose utilizing genes

| | | | | Yield g | |
|---|---|---|---|---|---|
| | | | | cadaverine | |
| | OD | Cadaverine | residual | per g sugar | lysine |
| strain | 600 nm | g/l | sugar | utilized | g/l |
| LU14635 | 25.50 | 8.04 | 6.85 | 0.15 | 0.93 |
| delta pG | | | | | |
| 60 g/l glucose | | | | | |
| LU14635 pG | 6.29 | 1.23 | 46.87 | 0.09 | 0.41 |
| 50 g/L xylose | | | | | |
| LU14635 pG | 27.50 | 8.76 | 6.75 | 0.18 | 0.76 |
| Xyl-Operon | | | | | |
| E. coli 60 g/l | | | | | |
| glucose | | | | | |
| LU14635 pG | 48.40 | 10.67 | 0.21 | 0.22 | 0.23 |
| Xyl-Operon | | | | | |
| E. coli 50 g/L | | | | | |
| xylose | | | | | |

The experiment showed that expression of the xylose utilizing genes lead to a good growth on xylose, while the control strain LU14635 pG, which grew well on glucose and produced cadaverine upon the addition of 50 g/L xylose did hardly grow or produce cadaverine. This shows that xylose cannot be converted by the C. glutamicum strain in the absence of the heterologous genes. The OD as a measure of cell growth showed the highest values when xylose was added to the medium, reaching even higher values and therefore cell numbers compared to the glucose addition to the medium. Also the cadaverine concentration was the highest in the experiment carrying the xylose utilizing genes on the plasmid pG xylose operon. The yield value (g of cadaverine per g of sugar utilized) obtained from this experiment also was increased over the yield in the case of added glucose. This data shows that the yield for the cadaverine production as well as the growth can be improved when the genes for xylose utilization are expressed in a cadaverine producing strain. This data also shows that C. glutamicum can be successfully used a s strain for converting pentoses to produce fine chemicals such as cadaverine.

### Example 8

### Strain Construction of for the Utilisation of C5 Sugars (Pentoses) for the Production of DAP with the Xylose Utilisation Genes Derived from Bacillus subtilis

The Bacillus subtilis genes for the utilisation of xylose, that is the xylose isomerase and the xylulose kinase, were fused to the PSOD promotor from C. glutamicum by PCR reaction, amplified by PCR and inserted into the vector pG into the restriction site XhoI and MluI to yield the vector pG PSOD xyl AB B. subtilis. The sequence of the vector is SEQ ID NO: 40. The plasmid was transformed into the strain LU14635 as well as the empty control plasmid pG, which did not contain the xylose utilization genes yielding the strains LU14635 pG and the strain LU14635 pG Pgro xylAB B. subtilis.

### Cadaverine Production in Shaking Flask Culture

Shake flask experiments were conducted as follows:

Cadaverine production in shaking flask culture of the aforementioned strains. The strain LU14635 pG and pG Pgro xylAB B. subtilis were analyzed for cadaverine production. Shaking flask experiments were performed with the recombinant strains with the plasmid to overexpress the xylose operon genes to test the cadaverine production. The strains were pre-cultured on CM agar with kanamycin 25 μg/ml overnight at 30° C. Cultured cells were harvested in a microtube containing 1.5 ml of 0.9% NaCl and cell density was determined by the absorbance at 610 nm following vortex. For the main culture suspended cells were inoculated to reach 1.5 of initial OD into 10 ml of the MDAP medium with added Kanamycin at a concentration of 25 μg/ml contained in an autoclaved 100 ml of Erlenmeyer flask. Main culture was performed on a rotary shaker (Infors AJ118, Bottmingen, Switzerland) with 200 rpm for 48-78 hours at 30° C. The concentrations of cadaverine and lysine and residual sugars were determined using HPLC (Agilent 1100 Series LC system). It was found that the strain LU14635 pG Pgro xylAB B. subtilis but not the strain LU14635 pG grew well on xylose as the single carbon source. The strain LU14635 pG Pgro xylAB B. subtilis upon growth on xylsoe as a single carbon source produces significant amounts of cadaverine while the control LU14635 pG does not produce cadaverine to a significant amount.

### Example 9

Strain construction secretion of a lysine decarboxylase. The E. coli Idc gene for the decarboxylation is fused to the PSOD promotor from C. glutamicum and to the signal sequence of the alpha amylase of Streptomyces coelicolor by PCR fusion. This fused gene then is cloned into the pCLIK vector to yield pCLIK PSOD Idc alpha-amylase sigseq (SEQ ID No. 41). This plasmid ins transformed into to yield the strain LU14635 pCLIK PSOD Idc alpha amy sigseq. Cadaverine production in shaking flask culture of the aforementioned strains. The strain LU14635 pCLIK and pCLIK PSOD Idc alpha amy sigseq are analyzed for cadaverine production. Shaking flask experiments are performed with the recombinant strains with the plasmid to overexpress the ldc which is secreted by C. glutamicum to test the cadaverine production. The strains are pre-cultured on CM agar with kanamycin 25 μg/ml overnight at 30° C. Cultured cells are harvested in a microtube containing 1.5 ml of 0.9% NaCl and cell density is determined by the absorbance at 610 nm following vortex. For the main culture suspended cells are inoculated to reach 1.5 of initial OD into 10 ml of the MDAP medium with added kanamycin at a concentration of 25 μg/ml contained in an autoclaved 100 ml of Erlenmeyer flask. Main culture is performed on a rotary shaker (Infors AJ118, Bottmingen, Switzerland) with 200 rpm for 48-78 hours at 30° C. The concentrations of cadaverine and lysine and residual sugars are determined using HPLC (Agilent 1100 Series LC system). It is found that the strain LU14635 pCLIK PSOD Idc alpha amy sigseq but not the strain LU14635 pCLIK produces no lysine in the cell supernatent and that the strain LU14635 pCLIK PSOD Idc alpha amy sigseq produces more cadaverine that the strain LU14635 pCLIK.

TABLE 4
Gene/protein descriptions and annotation
information, mutation information

| | | | Potential |
|---|---|---|---|
| | Accession | | mutation |
| | number | Accession number | in protein |
| Gene description | gene sequence | protein sequence | sequence |
| PEPCK | AJ269506 | NP_602055 | none |
| ddh | AAD09519 | AAE04861 | none |
| dapB | AAT49281 | AAW06582 | none |
| argS | AAT49283 | AAW06588 | none |
| lysA | AAD09520 | AAE04862 | none |
| Apartokinase ask, | X57226.1 | CAA40502.1 | T311 to I |
| lysC | | | |
| Homoserine de- | Cgl1183 | BAB98576.1 | V59 to A |
| hydrogenase | | | |
| Pyruvat carboxylase | AF038548 | AAB92588.1 | P458 to S |
| pycA | | | |
| Succinyl CoA- | BX927152 | A4QGW4_CORGB | P17 to S |
| synthase | | | G39 to E |
| | | | A165 to T |
| G6PDH | GeneID: 1019544 | NP_600790 | A243 to T |
| MMCM | GeneID: 1019502 | NC_003450.3 | P75 to S |
| | | | G413 to E |
| | | | A417 to V |
| ldc | FB656638 | CAR96105.1 | none |
| acetyltransferase | BX927152 | NP_600742 | none |
| lysE | | CAA65324.2 | none |

TABLE 5
Plasmid list

| Plasmid | Plasmid name | Use/Insert description |
|---|---|---|
| A | pInt sacB delta PEPCK | Deletion of PEPCK |
| B | pInt sacB 2x ddh | Duplication of ddh |
| C | pInt sacB LysC T311I | Introduction of 311I mutation |
| D | pInt sacB PSOD dapB | PSOD promotor to express |
| | | dapB |
| E | pK19 sacB 2x argS lysA | Gene duplication of argS |
| | | and the lysA gene |
| F | pInt sacB delta hom | Deletion of hom/hsdh |
| G | pInt sacB delta pycA | deleted pyruvate carboxylase |
| | | gene pycA |
| H | pInt sacB hom* | Introduction of mutated |
| | | hom gene |
| I | pInt sacB pycA* | Introduction of mutated |
| | | pyruvate carboxylase |
| | | gene |
| J | pInt sacB PSOD pycA | PSOD promotor to express |
| | | pycA |
| K | pInt sacB delta G6PDH | deletion of the glucose-6- |
| | | P-dehydrogenase gene |
| | | (G6PDH) |
| L | pInt sacB G6PDH* | Introduction of mutated |
| | | G6PDH gene |
| M | pInt sacB delta succ syn | deleted succinyl CoA synthase |
| | | beta chain |
| N | pInt sacB succ syn* | Introduction of mutated |
| | | succinyl CoA synthase |
| | | beta chain gene |
| O | pInt sacB delta MMCM | Deletion of methylmalonyl |
| | | CoA Mutase |
| P | pInt sacB MMCM* | Introduction of mutated |
| | | methylmalonyl CoA Mutase |
| | | gene |
| Q | pInt sacB PSOD G6PDH | PSOD promotor to express |
| | | the G6PDH gene |
| R | pInt sacB ldc bioD | Introduction of the E. coli |
| | | ldc gene with native promotor |
| | | into the bioD locus |
| S | pInt sacB delta RXA02214 | Deletion of acetyltransferase |
| T | pInt sacB delta lysE | Deletion of lysine exporter |
| | | lysE |
| U | pCKLIK SOD ldc sec | Secretion of the lysine |
| | | decarboxylase gene |

1. A microorganism comprising:
a) an intracellular lysine decarboxylase activity and an enhanced lysine import activity;
b) an intracellular and an extracellular lysine decarboxylase activity; or
c) an intracellular and an extracellular lysine decarboxylase activity and an enhanced lysine import activity.

2. The microorganism of claim 1, wherein the intracellular lysine decarboxylase activity, the extracellular lysine decarboxylase activity, or the intracellular and the extracellular lysine decarboxylase activity is at least partly due to expression of one or more lysine decarboxylase polypeptides comprising an amino acid sequence being at least 80% identical to SEQ ID NO: 1 or 2.

3.-4. (canceled)

5. The microorganism of claim 1, wherein the enhanced lysine import activity is due to a decreased lysine exporter activity, an enhanced lysine permease activity, an enhanced lysine/cadaverin antiporter activity, or any combination thereof.

6. The microorganism of claim 1, wherein the enhanced lysine import activity is due to decreased activity of a lysine exporter polypeptide comprising an amino acid sequence which has at least 80% sequence identity to SEQ ID NO: 3 or 4.

7. (canceled)

8. The microorganism of claim 1, wherein the enhanced lysine import activity is due to recombinant expression of:
a. at least one polypeptide comprising an amino acid sequence being at least 80% identical to SEQ ID NO: 8 and having lysine permease activity,
b. at least one polypeptide comprising an amino acid sequence being at least 80% identical to SEQ ID NO: 9 and having lysine/cadaverin antiporter activity, or
c. at least one polypeptide comprising an amino acid sequence being at least 80% identical to SEQ ID NO: 8 and having lysine permease activity, and at least one polypeptide comprising an amino acid sequence being at least 80% identical to SEQ ID NO: 9 and having lysine/cadaverin antiporter activity.

9. The microorganism of claim 1, having a high lysine production capacity.

10. The microorganism of claim 1, wherein the microorganism has no or a decreased acetylcadaverine-forming activity.

11. The microorganism of claim 10, wherein the acetylcadaverine-forming activity is decreased by decreasing the activity of an acetylcadaverine-forming polypeptide comprising an amino acid sequence being at least 80% identical to SEQ ID NO: 11.

12. The microorganism of claim 1, wherein the microorganism has no or a decreased aminopropylcadaverine-forming activity.

13. The microorganism of claim 12, wherein the aminopropylcadaverine-forming activity is decreased by decreasing the activity of an aminopropyl-cadaverine-forming polypeptide comprising an amino acid sequence being at least 80% identical to SEQ ID NO: 12.

14. The microorganism claim 1, wherein the microorganism has no or a decreased homoserine dehydrogenase activity.

15. The microorganism of claim 14, wherein the homoserine dehydrogenase activity is decreased by decreasing the activity of a homoserine dehydrogenase polypeptide comprising an amino acid sequence being at least 80% identical to SEQ ID NO: 13, 14 or 15.

16. The microorganism of claim 1, wherein the microorganism has no or a decreased lysine degradation activity.

17. The microorganism of claim 16, wherein the lysine degradation activity is decreased by decreasing the activity of at least one lysine hydroxylase polypeptide comprising an amino acid sequence being at least 80% identical to SEQ ID NO: 16.

18. The microorganism of claim 1, wherein the microorganism has a deregulated spermidine forming or uptake activity, or a deregulated putrescine forming or uptake activity, or both.

19. The microorganism of claim 18, wherein the spermidine forming or uptake activity or the putrescine forming or uptake activity is decreased by decreasing the activity of:
a. at least one spermidine forming polypeptide comprising an amino acid sequence being at least 80% identical to SEQ ID NO: 12,
b. at least one putrescine forming polypeptide comprising an amino acid sequence being at least 80% identical to SEQ ID NO:17,
c. at least one putrescine forming polypeptide comprising an amino acid sequence being at least 80% identical to SEQ ID NO:18,
d. at least one spermidine/putrescine transporting polypeptide comprising an amino acid sequence being at least 80% identical to SEQ ID NO: 19, or
e. any combination of a, b, c and d.

20. The microorganism of claim 1, wherein the microorganism belongs to the Glade Eubacteria, the genus Corynebacterium, or the genus Escherichia, or wherein the microorganism is Corynebacterium glutamicum or Escherichia coli.

21.-23. (canceled)

24. A cadaverine production system comprising the microorganism of claim 1 and a culture medium suitable to grow the microorganism.

25. The cadaverine production system of claim 24, wherein the culture medium comprises lysine.

26. A process for production of cadaverine, comprising fermenting the microorganism of claim 1 in a culture medium.

27. The process of claim 26, wherein the culture medium comprises lysine.

28. The process of claim 26, wherein the concentration (mol/l) of cadaverine in the culture medium is:
a. at least 1.2 times higher than the concentration (mol/l) of acetylcadaverine, or
b. at least 1.2 times higher than the concentration (mol/l) of aminopropylcadaverine.

29. (canceled)

30. A culture medium comprising cadaverine at a concentration (mol/l) that is:
a. at least 1.2 times higher than the concentration (mol/l) of acetylcadaverine, or
b. at least 1.2 times higher than the concentration (mol/l) of aminopropylcadaverine.

31. The culture medium of claim 30, wherein the concentration (mol/l) of cadaverine is at least 1.2 times higher than the concentration (mol/l) of acetylcadaverine.

32. A culture medium comprising at least 0.25 mol/l cadaverine but comprising
a. not more than 0.1 mol/l of acetylcadaverine, or
b. not more than 0.1 mol/l of aminopropylcadaverine.

33.-35. (canceled)

36. A process for recovery of cadaverine, comprising utilizing the culture medium of claim 30.

37. The process of claim 26, further comprising purifying the cadaverine from the culture medium.


SEQUENCE LISTING (42 sequences)

SEQ ID NO: 1 - protein, 713 aa, Escherichia coli
MNIIAIMGPHGVFYKDEPIKELESALVAQGFQIIWPQNSVDLLKFIEHNPRICGVIFDWDEYSLDLCSDINQLNEYLPLYAFINTHSTMDVSVQDMRMALWFFEYALGQAEDIAIRMRQYTDEYLDNITPPFTKALFTYVKERKYTFCTPGHMGGTAYQKSPVGCLFYDFFGGNTLKADVSISVTELGSLLDHTGPHLEAEEYIARTFGAEQSYIVTNGTSTSNKIVGMYAAPSGSTLLIDRNCHKSLAHLLMMNDVVPVWLKPTRNALGILGGIPRREFTRDSIEEKVAATTQAQWPVHAVITNSTYDGLLYNTDWIKQTLDVPSIHFDSAWVPYTHFHPIYQGKSGMSGERVAGKVIFETQSTHKMLAALSQASLIHIKGEYDEEAFNEAFMMHTTTSPSYPIVASVETAAAMLRGNPGKRLINRSVERALHFRKEVQRLREESDGWFFDIWQPPQVDEAECWPVAPGEQWHGFNDADADHMFLDPVKVTILTPGMDEQGNMSEEGIPAALVAKFLDERGIVVEKTGPYNLLFLFSIGIDKTKAMGLLRGLTEFKRSYDLNLRIKNMLPDLYAEDPDFYRNMRIQDLAQGIHKLIRKHDLPGLMLRAFDTLPEMIMTPHQAWQRQIKGEVETIALEQLVGRVSANMILPYPPGVPLLMPGEMLTKESRTVLDFLLMLCSVGQHYPGFETDIHGAKQDEDGVYRVRVLKMAG

SEQ ID NO: 2 - protein, 715 aa, Escherichia coli
MNVIAILNHMGVYFKEEPIRELHRALERLNFQIVYPNDRDDLLKLIENNARLCGVIFDWDKYNLELCEEISKMNENLPLYAFANTYSTLDVSLNDLRLQISFFEYALGAAEDIANKIKQTTDEYINTILPPLTKALFKYVREGKYTFCTPGHMGGTAFQKSPVGSLFYDFFGPNTMKSDISISVSELGSLLDHSGPHKEAEQYIARVFNADRSYMVTNGTSTANKIVGMYSAPAGSTILIDRNCHKSLTHLMMMSDVTPIYFRPTRNAYGILGGIPQSEFQHATIAKRVKETPNATWPVHAVITNSTYDGLLYNTDFIKKTLDVKSIHFDSAWVPYTNFSPIYEGKCGMSGGRVEGKVIYETQSTHKLLAAFSQASMIHVKGDVNEETFNEAYMMHTTTSPHYGIVASTETAAAMMKGNAGKRLINGSIERAIKFRKEIKRLRTESDGWFFDVWQPDHIDTTECWPLRSDSTWHGFKNIDNEHMYLDPIKVTLLTPGMEKDGTMSDFGIPASIVAKYLDEHGIVVEKTGPYNLLFLFSIGIDKTKALSLLRALTDFKRAFDLNLRVKNMLPSLYREDPEFYENMRIQELAQNIHKLIVHHNLPDLMYRAFEVLPTMVMTPYAAFQKELHGMTEEVYLDEMVGRINANMILPYPPGVPLVMPGEMITEESRPVLEFLQMLCEIGAHYPGFETDIHGAYRQADGRYTVKVLKEESKK

SEQ ID NO: 3 - protein, 233 aa, Corynebacterium glutamicum
MEIFITGLLLGASLLLSIGPQNVLVIKQGIKREGLIAVLLVCLISDVFLFIAGTLGVDLLSNAAPIVLDIMRWGGIAYLLWFAVMAAKDAMTNKVEAPQIIEETEPTVPDDTPLGGSAVATDTRNRVRVEVSVDKQRVWVKPMLMAIVLTWLNPNAYLDAFVFIGGVGAQYGDTGRWIFAAGAFAASLIWFPLVGFGAAALSRPLSSPKVWRWINVVVAVVMTALAIKLMLMG

SEQ ID NO: 4 - protein, 299 aa, Escherichia coli
MFSGLLIILVPLIVGYLIPLRQQAALKVINQLLSWMVYLILFFMGISLAFLDNLASNLLAILHYSAVSITVILLCNIAALMWLERGLPWRNHHQQEKLPSRIAMALESLKLCGVVVIGFAIGLSGLAFLQHATEASEYTLILLLFLVGIQLRNNGMTLKQIVLNRRGMIVAVVVVVSSLIGGLINAFILDLPINTALAMASGFGWYSLSGILLTESFGPVIGSAAFFNDLARELIAIMLIPGLIRRSRSTALGLCGATSMDFTLPVLQRTGGLDMVPAAIVHGFILSLLVPILIAFFSA

SEQ ID NO: 5 - DNA, 873 bp, Corynebacterium glutamicum
atgaaccccattcaactggacactttgctctcaatcattgatgaaggcagcttcgaaggcgcctccttagccctttccatttccccctcggcggtgagtcagcgcgttaaagctctcgagcatcacgtgggtcgagtgttggtatcgcgcacccaaccggccaaagcaaccgaagcgggtgaagtccttgtgcaagcagcgcggaaaatggtgttgctgcaagcagaaactaaagcgcaactatctggacgccttgctgaaatcccgttaaccatcgccatcaacgcagattcgctatccacatggtttcctcccgtgttcaacgaggtagcttcttggggtggagcaacgctcacgctgcgcttggaagatgaagcgcacacattatccttgctgcggcgtggagatgttttaggagcggtaacccgtgaagctaatcccgtggcgggatgtgaagtagtagaacttggaaccatgcgccacttggccattgcaaccccctcattgcgggatgcctacatggttgatgggaaactagattgggctgcgatgcccgtcttacgcttcggtcccaaagatgtgcttcaagaccgtgacctggacgggcgcgtcgatggtcctgtggggcgcaggcgcgtatccattgtcccgtcggcggaaggttttggtgaggcaattcgccgaggccttggttggggacttcttcccgaaacccaagctgctcccatgctaaaagcaggagaagtgatcctcctcgatgagatacccattgacacaccgatgtattggcaacgatggcgcctggaatctagatctctagctagactcacagacgccgtcgttgatgcagcaatcgagggattgcggccttag

SEQ ID NO: 6 - protein, 290 aa, Corynebacterium glutamicum
MNPIQLDTLLSIIDEGSFEGASLALSISPSAVSQRVKALEHHVGRVLVSRTQPAKATEAGEVLVQAARKMVLLQAETKAQLSGRLAEIPLTIAINADSLSTWFPPVFNEVASWGGATLTLRLEDEAHTLSLLRRGDVLGAVTREANPVAGCEVVELGTMRHLAIATPSLRDAYMVDGKLDWAAMPVLRFGPKDVLQDRDLDGRVDGPVGRRRVSIVPSAEGFGEAIRRGLGWGLLPETQAAPMLKAGEVILLDEIPIDTPMYWQRWRLESRSLARLTDAVVDAAIEGLRP

SEQ ID NO: 7 - DNA, 1470 bp, Escherichia coli
atggtttccgaaactaaaaccacagaagcgccgggcttacgccgtgaattaaaggcgcgtcacctgacgatgattgccattggcggttccatcggtacaggtctttttgttgcctctggcgcaacgatttctcaggcaggtccgggcggggcattgctctcgtatatgctgattggcctgatggtttacttcctgatgaccagtctcggtgaactggctgcatatatgccggtttccggttcgtttgccacttacggtcagaactatgttgaagaaggctttggcttcgcgctgggctggaactactggtacaactgggcggtgactatcgccgttgacctggttgcagctcagctggtcatgagctggtggttcccggatacaccgggctggatctggagtgcgttgttcctcggcgttatcttcctgctgaactacatctcagttcgtggctttggtgaagcggaatactggttctcactgatcaaagtcacgacagttattgtctttatcatcgttggcgtgctgatgattatcggtatcttcaaaggcgcgcagcctgcgggctggagcaactggacaatcggcgaagcgccgtttgctggtggttttgcggcgatgatcggcgtagctatgattgtcggcttctctttccagggaaccgagctgatcggtattgctgcaggcgagtccgaagatccggcgaaaaacattccacgcgcggtacgtcaggtgttctggcgaatcctgttgttctatgtgttcgcgatcctgattatcagcctgattattccgtacaccgatccgagcctgctgcgtaacgatgttaaagacatcagcgttagtccgttcaccctggtgttccagcacgcgggtctgctctctgcggcggcggtgatgaacgcagttattctgacggcggtgctgtcagcgggtaactccggtatgtatgcgtctactcgtatgctgtacaccctggcgtgtgacggtaaagcgccgcgcattttcgctaaactgtcgcgtggtggcgtgccgcgtaatgcgctgtatgcgacgacggtgattgccggtctgtgcttcctgacctccatgtttggcaaccagacggtatacctgtggctgctgaacacctccgggatgacgggttttatcgcctggctggggattgccattagccactatcgcttccgtcgcggttacgtattgcagggacacgacattaacgatctgccgtaccgttcaggtttcttcccactggggccgatcttcgcattcattctgtgtctgattatcactttgggccagaactacgaagcgttcctgaaagatactattgactggggcggcgtagcggcaacgtatattggtatcccgctgttcctgattatttggttcggctacaagctgattaaaggaactcacttcgtacgctacagcgaaatgaagttcccgcagaacgataagaaataa

SEQ ID NO: 8 - protein, 489 aa, Escherichia coli
MVSETKTTEAPGLRRELKARHLTMIAIGGSIGTGLFVASGATISQAGPGGALLSYMLIGLMVYFLMTSLGELAAYMPVSGSFATYGQNYVEEGFGFALGWNYWYNWAVTIAVDLVAAQLVMSWWFPDTPGWIWSALFLGVIFLLNYISVRGFGEAEYWFSLIKVTTVIVFIIVGVLMIIGIFKGAQPAGWSNWTIGEAPFAGGFAAMIGVAMIVGFSFQGTELIGIAAGESEDPAKNIPRAVRQVFWRILLFYVFAILIISLIIPYTDPSLLRNDVKDISVSPFTLVFQHAGLLSAAAVMNAVILTAVLSAGNSGMYASTRMLYTLACDGKAPRIFAKLSRGGVPRNALYATTVIAGLCFLTSMFGNQTVYLWLLNTSGMTGFIAWLGIAISHYRFRRGYVLQGHDINDLPYRSGFFPLGPIFAFILCLIITLGQNYEAFLKDTIDWGGVAATYIGIPLFLIIWFGYKLIKGTHFVRYSEMKFPQNDKK

SEQ ID NO: 9 - protein, 444 aa, Escherichia coli
MSSAKKIGLFACTGVVAGNMMGSGIALLPANLASIGGIAIWGWIISIIGAMSLAYVYARLATKNPQQGGPIAYAGEISPAFGFQTGVLYYHANWIGNLAIGITAVSYLSTFFPVLNDPVPAGIACIAIVWVFTFVNMLGGTWVSRLTTIGLVLVLIPVVMTAIVGWHWFDAATYAANWNTADTTDGHAIIKSILLCLWAFVGVESAAVSTGMVKNPKRTVPLATMLGTGLAGIVYIAATQVLSGMYPSSVMAASGAPFAISASTILGNWAAPLVSAFTAFACLTSLGSWMMLVGQAGVRAANDGNFPKVYGEVDSNGIPKKGLLLAAVKMTALMILITLMNSAGGKASDLFGELTGIAVLLTMLPYFYSCVDLIRFEGVNIRNFVSLICSVLGCVFCFIALMGASSFELAGTFIVSLIILMFYARKMHERQSHSMDNHTASNAH

SEQ ID NO: 10 - DNA, 558 bp, Corynebacterium glutamicum
atgagtcccaccgttttgcctgctacacaagctgacttccctaagatcgtcgatgttctggttgaagcattcgccaacgatccagcatttttacgatggatcccgcagccggaccccggttcagcaaagcttcgagcacttttcgaactgcagattgagaagcagtatgcagtggcgggaaatattgatgtcgcgcgtgattctgagggagaaatcgtcggcgtcgcgttatgggatcggccagatggtaatcacagtgccaaagatcaagcagcgatgctcccccggctcgtctccattttcgggatcaaggctgcgcaggtggcgtggacggatttgagttcggctcgtttccaccccaaattcccccattggtacctctacaccgtggcaacatctagttctgcccgtggaacgggtgttggcagtgcgcttcttaatcacggaatcgctcgcgcgggtgatgaagctatctatttggaggcgacgccgactcgtgcggctcaactatataaccgtctgggattcgtgcccttgggttatatccccctcagatga

SEQ ID NO: 11 - protein, 185 aa, Corynebacterium glutamicum
MSPTVLPATQADFPKIVDVLVEAFANDPAFLRWIPQPDPGSAKLRALFELQIEKQYAVAGNIDVARDSEGEIVGVALWDRPDGNHSAKDQAAMLPRLVSIFGIKAAQVAWTDLSSARFHPKFPHWYLYTVATSSSARGTGVGSALLNHGIARAGDEAIYLEATPTRAAQLYNRLGFVPLGYIPLR

SEQ ID NO: 12 - protein, 288 aa, Escherichia coli
MAEKKQWHETLHDQFGQYFAVDNVLYHEKTDHQDLIIFENAAFGRVMALDGVVQTTERDEFIYHEMMTHVPLLAHGHAKHVLIIGGGDGAMLREVTRHKNVESITMVEIDAGVVSFCRQYLPNHNAGSYDDPRFKLVIDDGVNFVNQTSQTFDVIISDCTDPIGPGESLFTSAFYEGCKRCLNPGGIFVAQNGVCFLQQEEAIDSHRKLSHYFSDVGFYQAAIPTYYGGIMTFAWATDNDALRHLSTEIIQARFLASGLKCRYYNPAIHTAAFALPQYLQDALASQPS

SEQ ID NO: 13 - protein, 445 aa, Corynebacterium glutamicum
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVGVLAELASLFSEQGISLRTIRQEERDDDARLIVVTHSALESDLSRTVELLKAKPVVKAINSVIRLERD

SEQ ID NO: 14 - protein, 810 aa, Escherichia coli
MSVIAQAGAKGRQLHKFGGSSLADVKCYLRVAGIMAEYSQPDDMMVVSAAGSTTNQLINWLKLSQTDRLSAHQVQQTLRRYQCDLISGLLPAEEADSLISAFVSDLERLAALLDSGINDAVYAEVVGHGEVWSARLMSAVLNQQGLPAAWLDAREFLRAERAAQPQVDEGLSYPLLQQLLVQHPGKRLVVTGFISRNNAGETVLLGRNGSDYSATQIGALAGVSRVTIWSDVAGVYSADPRKVKDACLLPLLRLDEASELARLAAPVLHARTLQPVSGSEIDLQLRCSYTPDQGSTRIERVLASGTGARIVTSHDDVCLIEFQVPASQDFKLAHKEIDQILKRAQVRPLAVGVHNDRQLLQFCYTSEVADSALKILDEAGLPGELRLRQGLALVAMVGAGVTRNPLHCHRFWQQLKGQPVEFTWQSDDGISLVAVLRTGPTESLIQGLHQSVFRAEKRIGLVLFGKGNIGSRWLELFAREQSTLSARTGFEFVLAGVVDSRRSLLSYDGLDASRALAFFNDEAVEQDEESLFLWMRAHPYDDLVVLDVTASQQLADQYLDFASHGFHVISANKLAGASDSNKYRQIHDAFEKTGRHWLYNATVGAGLPINHTVRDLIDSGDTILSISGIFSGTLSWLFLQFDGSVPFTELVDQAWQQGLTEPDPRDDLSGKDVMRKLVILAREAGYNIEPDQVRVESLVPAHCEGGSIDHFFENGDELNEQMVQRLEAAREMGLVLRYVARFDANGKARVGVEAVREDHPLASLLPCDNVFAIESRWYRDNPLVIRGPGAGRDVTAGAIQSDINRLAQLL

SEQ ID NO: 15 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 16 - protein, 445 aa, Escherichia coli
MKKSVDFIGVGTGPFNLSIAALSHQIEELNCLFFDEHPHFSWHPGMLVPDCHMQTVFLKDLVSAVAPTNPYSFVNYLVKHKKFYRFLTSRLRTVSREEFSDYLRWAAEDMNNLYFSHTVENIDFDKKSRLFLVQTSRGEYFARNICLGTGKQPYLPPCVKHVTQSCFHASEMNLRRPDLSGKRITVVGGGQSGADLFLNALRGEWGEAAEINWVSRRNNFNALDEAAFADEYFTPEYISGFSGLKEDIRHQLLDEQKMTSDGITADSLLTIYRELYHRFEVLRKPRNIRLLPSRSVTTLESSGPGWKLLMEHHLDRGRESLESDVVIFATGYRSALPQILPSLMPLITMHDKNTFKVRDDFTLEWSGPKENNIFAVNASMQTHGIAEPQLSLMAWRSARILNRVLGRDLFDLSMPPALIQWRSGSRKKPQPEAAALTHYTTNIQE

SEQ ID NO: 17 - protein, 472 aa, Escherichia coli
METNIVEVENFVQQSEERRGSAFTQEVKRYLERYPNTQYVDVLLTDLNGCFRGKRIPVSSLKKLEKGCYFPASVFAMDILGNVVEEAGLGQEMGEPDRTCVPVLGSLTPSAADPEFIGQMLLTMVDEDGAPFDVEPRNVLNRLWQQLRQRGLFPVVAVELEFYLLDRQRDAEGYLQPPCAPGTDDRNTQSQVYSVDNLNHFADVLNDIDELAQLQLIPADGAVAEASPGQFEINLYHTDNVLEACDDALALKRLVRLMAEKHKMHATFMAKPYEEHAGSGMHIHISMQNNRGENVLSDAEGEDSPLLKKMLAGMIDLMPSSMALLAPNVNSYRRFQPGMYVPTQASWGHNNRTVALRIPCGDRHNHRVEYRVAGADANPYLVMAAIFAGILHGLDNELPLQEEVEGNGLEQEGLPFPIRQSDALGEFIENDHLRRYLGERFCHVYHACKNDELLQFERLITETEIEWMLKNA

SEQ ID NO: 18 - protein, 732 aa, Escherichia coli
MSKLKIAVSDSCPDCFTTQRECIYINESRNIDVAAIVLSLNDVTCGKLDEIDATGYGIPVFIATENQERVPAEYLPRISGVFENCESRREFYGRQLETAASHYETQLRPPFFRALVDYVNQGNSAFDCPGHQGGEFFRRHPAGNQFVEYFGEALFRADLCNADVAMGDLLIHEGAPCIAQQHAAKVFNADKTYFVLNGTSSSNKVVLNALLTPGDLVLFDRNNHKSNHHGALLQAGATPVYLETARNPYGFIGGIDAHCFEESYLRELIAEVAPQRAKEARPFRLAVIQLGTYDGTIYNARQVVDKIGHLCDYILFDSAWVGYEQFIPMMADCSPLLLDLNENDPGILVTQSVHKQQAGFSQTSQIHKKDSHIKGQQRYVPHKRMNNAFMMHASTSPFYPLFAALNINAKMHEGVSGRNMWMDCVVNGINARKLILDNCQHIRPFVPELVDGKPWQSYETAQIAVDLRFFQFVPGEHWHSFEGYAENQYFVDPCKLLLTTPGIDARNGEYEAFGVPATILANFLRENGVVPEKCDLNSILFLLTPAEDMAKLQQLVALLVRFEKLLESDAPLAEVLPSIYKQHEERYAGYTLRQLCQEMHDLYARHNVKQLQKEMFRKEHFPRVSMNPQEANYAYLRGEVELVRLPDAEGRIAAEGALPYPPGVLCVVPGEIWGGAVLRYFSALEEGINLLPGFAPELQGVYIEEHDGRKQVWCYVIKPRDAQSTLLKGEKL

SEQ ID NO: 19 - protein, 439 aa, Escherichia coli
MSQAKSNKMGVVQLTILTMVNMMGSGIIMLPTKLAEVGTISIISWLVTAVGSMALAWAFAKCGMFSRKSGGMGGYAEYAFGKSGNFMANYTYGVSLLIANVAIAISAVGYGTELLGASLSPVQIGLATIGVLWICTVANFGGARITGQISSITVWGVIIPVVGLCIIGWFWFSPTLYVDSWNPHHAPFFSAVGSSIAMTLWAFLGLESACANTDVVENPERNVPIAVLGGTLGAAVIYIVSTNVIAGIVPNMELANSTAPFGLAFAQMFTPEVGKVIMALMVMSCCGSLLGWQFTIAQVFKSSSDEGYFPKIFSRVTKVDAPVQGMLTIVIIQSGLALMTISPSLNSQFNVLVNLAVVTNIIPYILSMAALVIIQKVANVPPSKAKVANFVAFVGAMYSFYALYSSGEEAMLYGSIVTFLGWTLYGLVSPRFELKNKHG

SEQ ID NO: 20 - DNA, 6527 bp, Corynebacterium glutamicum
gatcccaggctgagtgggatcgcatggcggaggatcttgttgaagccggtaccctcatcaagctcaacgaggaaaagcgtccgaacagctacctagctcgttccaacccatctgacgttgcgcgcgttgagtcccgcaccttcatctgctccgagaaggaagaagatgctggcccaaccaacaactgggctccaccacaggcaatgaaggacgaaatgtccaagcattacgctggttccatgaaggggcgcaccatgtacgtcgtgcctttctgcatgggtccaatcagcgatccggaccctaagcttggtgtgcagctcactgactccgagtacgttgtcatgtccatgcgcatcatgacccgcatgggtattgaagcgctggacaatgtttaagtttagtggatggggcagaactggattgacatgggtaacaagggtggcgacaagatgccatccatcttcctggtcaactggttccgccgtggcgaagatggacgcttcctgtggcctggcttcggcgacaactctcgcgttctgaagtgggtcatcgaccgcatcgaaggccacgttggcgcagacgagaccgttgttggacacaccgctaaggccgaagacctcgacctcgacggcctcgacaccccaattgaggatgtcaaggaagcactgaccgctcctgcagagcagtgggcaaacgacgttgaagacaacgccgagtacctcactttcctcggaccacgtgttcctgcagaggttcacagccagttcgatgctctgaaggcccgcatttcagcagctcacgcttaaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagag

SEQ ID NO: 21 - DNA, 8257 bp, Corynebacterium glutamicum
aaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgacttcctcttggtccagcgaagacaccctctgaaaaggctaaaagaggcaaggaaaccacactgtttccttgcccctcgagctaaattagacgtcgcgtgcgatcagatcgtccaagttctctggggagagcaggtatggagcaacttcgaggacggtgaaagctccgctttggccctgctgcttcatgcggtgagctgcgcgaccgaaagcgatctgtgaggaagcggtgaaatctgggtttcggtccagcttgaggatgtattccacggtgtggttgaagccaccggtgtcgccggtggtaatcacgtggccaccgtgtggcatgccggtgtgctcggagtcgaaggttgcttcgtcgatgaagttgacttcgacttcgtagccaacgaagtaatcaggcatggtgcggatgtcgttttcgatgcgctcgtgatcggccgcgtcggcaaccacgaagcattggcgcttgtgggtttgctttccggtaaggtcgccggcttcgccgcggcgggccttttccagggcgtcttcggatgggagggtgtactggactgccttttgaacgccagggatgcgtcgcaaagcatcggagtggccctgtgacaaacctgggccccagaaggtgtgctgctggtgctcggctaagactgccgctgcgtagacgcggttgatggagaacattcctggatcccagccggtagagaccagtgcaacgttgccggctgcggtggcggcttcgttcatgacctggcggtggcgtgggatgtcgcggtggttgtcgtaggtgtctacggtgcaggcgaactgcgcgaactttggtgcctgctcagggatgtcggtggcggagcccatgcacaggaacagcacgtccacgtcgtcggcgtgcttgtccacgtcggcgacatcaaagactggcgtctttgtgtcgagggtggcccggcgcgagaagattcctacaaggtccatgtcgggctgcttggcaataagcttttcgacgctgcgtcccaggtttccgtagcccacgatagctacgcggatgttggtcatgttcttgtaatcctccaaaattgtggtggcactgtcctggtcgagcttaccgagatgcatacttagatgatgattcagggacatctctttcatcaggaccgaaagcgaacgtttcgtattgttgagccttttggttccaccacggatgcgctgatctattttcatggctcccagcagtcaggatctgtggggcgcagcttcaccaacaggacttttgatccgttgccgttcatctagattcctcttggtccagcgaagacaccctctgaaaaggctaaaagaggcaaggaaaccacactgtttccttgcccctcgagctaaattagacgtcgcgtgcgatcagatcgtccaagttctctggggagagcaggtatggagcaacttcgaggacggtgaaagctccgctttggccctgctgcttcatgcggtgagctgcgcgaccgaaagcgatctgtgaggaagcggtgaaatctgggtttcggtccagcttgaggatgtattccacggtgtggttgaagccaccggtgtcgccggtggtaatcacgtggccaccgtgtggcatgccggtgtgctcggagtcgaaggttgcttcgtcgatgaagttgacttcgacttcgtagccaacgaagtaatcaggcatggtgcggatgtcgttttcgatgcgctcgtgatcggccgcgtcggcaaccacgaagcattggcgcttgtgggtttgctttccggtaaggtcgccggcttcgccgcggcgggccttttccagggcgtcttcggatgggagggtgtactggactgccttttgaacgccagggatgcgtcgcaaagcatcggagtggccctgtgacaaacctgggccccagaaggtgtgctgctggtgctcggctaagactgccgctgcgtagacgcggttgatggagaacattcctggatcccagccggtagagaccagtgcaacgttgccggctgcggtggcggcttcgttcatgacctggcggtggcgtgggatgtcgcggtggttgtcgtaggtgtctacggtgcaggcgaactgcgcgaactttggtgcctgctcagggatgtcggtggcggagcccatgcacaggaacagcacgtccacgtcgtcggcgtgcttgtccacgtcggcgacatcaaagactggcgtctttgtgtcgagggtggcccggcgcgagaagattcctacaaggtccatgtcgggctgcttggcaataagcttttcgacgctgcgtcccaggtttccgtagcccacgatagctacgcggatgttggtcatgttcttgtaatcctccaaaattgtggtggcactgtcctggtcgagcttaccgagatgcatacttagatgatgattcagggacatctctttcatcaggaccgaaagcgaacgtttcgtattgttgagccttttggttccaccacggatgcgctgatctattttcatggctcccagcagtcaggatctgtggggcgcagcttcaccaacaggacttttgatccgttgccgttcagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataa

SEQ ID NO: 22 - DNA, 8438 bp, Corynebacterium glutamicum
aattcgcccttcaccgcggctttggacatcactgctacgtagccaaacaatgcacccgtcacaagaccaaggatgagggctttgtccttctttaatacgtattccgcaagcagccacattccacccattactgcaacgccgactaaaagtactggaatccatcgatcgagtgggggtgggggtttccgggaagggggcgtcccaaaacgatcatgatgcccacggctacggtgaggagggtagcccagaagatttcagttcggcgtagtcggtagccattgaatcgtgctgagagcggcagcgtgaacatcagcgacaggacaagcactggttgcactaccaagagggtgccgaaaccaagtgctactgtttgtaagaaatatgccagcatcgcggtactcatgcctgcccaccacatcggtgtcatcagagcattgagtaaaggtgagctccttagggagccatcttttggggtgcggagcgcgatccggtgtctgaccacggtgccccatgcgattgttaatgccgatgctagggcgaaaagcacggcgagcagattgctttgcacttgattcagggtagttgactaaagagttgctcgcgaagtagcacctgtcacttttgtctcaaatattaaatcgaatatcaatatatggtctgtttattggaacgcgtcccagtggctgagacgcatccgctaaagccccaggaagggcgaattctgcagatatccatcacactggcggccgctcgagcatgcatctagcttatcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccgtggcacggaaatcgaggtagaagacattactcaggcaaccgaaagggcgaattccgtggcacggaaatcgaggtagaagacattactcaggcaaccgaaagggcgaattcgcccttgcggcgcaggattttctaaaacaggatgcctgccacctttaagcgcctcatcagcggtaaccatcacgggttcgggtgcgaaaaaccatgccataacaggaatgttcctttcgaaaattgaggaagccttatgcccttcaaccctacttagctgccaattattccgggcttgtgacccgctacccgataaataggtcggctgaaaaatttcgttgcaatataaacaaaaaggcctatcattgggaggtgtcgcaccaagtacttttgcgaagcgccatctgacggattttcaaaagatgtatatgctcggtgcggaaacctacgaaaggattttttacccgtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttgcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcatcttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaaagttttaaaggagtagttttacaatgaccaccatcgcagttgttggtgcaaccggccaggtcggccaggttatgcgcacccttttggaagagcgcaatttcccagctgacactgttcgtttctttgcttccccacgttccgcaggccgtaagattgaattcgccctttcggttgcctgagtaatgtcttctacctcgatttccgtgccacggaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgctcggtacccatcggcattttcttttgcgtttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagtaacggccgccagtgtgctgg

SEQ ID NO: 23 - DNA, 8310 bp, Corynebacterium glutamicum
aaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttcgaagagaccggcgcaaacatctccatcgaagatgacggcaccgtgttcatctctgcagctgacggcgcatctgctgaagcggcgatcgaaaagatcaacgctctggcgaacccacagctgccaaaggttggcgagcgcttcctcggaaccgtcgtcaagaccaccgcattcggagcattcgtttccttgctcccaggccgcgacggccttgttcacatctccaagctgggtaacggcaagcgagtagaaaaggtcgacgatgtggtgaaggttggcgagaagattcaggtcgaaatcgctgacatcgacaaccgcggcaagatctccttggtcccagttgttgaagaggactaattagttctggctagatcgggctagatcgggctaaaaataaatgtggcgctaaaaccttattgcgaggttttagcgccacatttttctatctgtagttgacggcgttccagcgctagtgaacgatgtccaccacaagcctggttggttcctgcagcaatgagaccgagtaatttcggggttgaccagatacaccaatgagaacttgggaacgggcttcaaaaatactggtgaagttgatgtcttcaacaatgcctgcaccaggatatgatccggtatcgatacctggaacgacaacctgatcaggatatccagtgccttgaatattgacgttgaggaaggaatcaccagccatctcaactggaagacctgacgcctgctgaattggatcagtggcccaatcgacccaccaaccaggttggccattaccggcgatatcaaaaacaactcgtgtgaacgtttcgtgctcggcaacgcggatgccagcgatcgacatatcggagtcaccaacttgagcctgctgcttctgatccatcgacggggaacccaacggcggcaaagcagtgggggaaggggagttggtggactctgaaccagtgggctctgaagtggtaggcgacggggcagcggcgcaggataagggcgaattctgcagatatccatcacactggcggccgctcgagcatgcaggtcgactctagaggatccccgggtaccgagctcgaattcgccctttaggcgacggggcgcggcgcaggattttctaaaacaggatgcctgccacctttaagcgcctcatcagcggtaaccatcacgggttcgggtgcgaaaaaccatgccataacaggaatgttcctttcgaaaattgaggaagccttatgcccttcaaccctacttagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgggaatcaaggttggcgttctcggagccaaaggccgtgttggtcaaactattgtggcagcagtcaatgagtccgacgatctggagcttgttgcagagatcggcgtcgacgatgatttgagccttctggtagacaacggcgctgaagttgtcgttgacttcaccactcctaacgctgtgatgggcaacctggagttctgcatcaacaacggcatttctgcggttgttggaaccacgggcttcgatgatgctcgtttggagcaggttcgcgactggcttgaaggaaaagacaatgtcggtgttctgatcgcacctaactttgctatctctgcggtgttgaccatggtcttttccaagcaggctgcccgcttcttcgaatcagctgaagttattgagctgcaccaccccaacaagctggatgcaccttcaggcaccgcgatccacactgctcagggcattgctgcggcacgcaaagaagcaggcatggacgcacagccagatgcgaccgagcaggcacttgagggttcccgtggcgcaagcgtagatggaatcccggttcatgcagtccgcatgtccggcatggttgctcacgagcaagttatctttggcacccagggtcagaccttgaccatcaagcaggactcctatgatcgcaactcatttgcaccaggtgtcttggtgggtgtgcgcaacattgcacagcacccaggcctagtcgtaggacttgagcattacctaggcctgtaaaggctcatttcagcagcgggtggaattttttaaaaggagcgtttaaaggctgtggccgaacaagttaaattgagcgtggagttgatagcgtgcagttcttttactccacccgctgatgttgagtggtcaactgatgttgagggcgcggaagcactcgtcgagtttgcgggtcgtgcctgctacgaaacttttgataagccgaaccctcgaactgcttccaatgctgcgtatctgcgccacatcatggaagtggggcacactgctttgcttgagcatgccaatgccacgatgtatatccgaggcatttctcggtccgcgacccatgaattggtccgacaccgccatttttccttctctcaactgtctcagcgtttcgtgcacagcggaaagggcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataa

SEQ ID NO: 24 - DNA, 12846 bp, Corynebacterium glutamicum
aaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgaaacccaaaaccgccctccacggcgaaggtgaagggcggggtcaggcgtcgaaaagcgttatgcctctagtgagaggatgtcgtcgagcgtttcgcggcgcagcatgaggcgggagctgccagcgcggacggacacgacggcgggccgtgtgaaggcgttgtagcgggagctcatggcgtagcagtatgcgccggtggctgcgagtgcaaggaagtcgccgctggtgatgtcagatgggtagatttcatcgttgatcaggatatcgccggattcgcagtgggagcccacgatgcgggtgcttactgggtctccttcggcgaagcgggatactacgcgggcgtcgtattcggagccgtagagtgctgggcggatgttgtcggacatgcctccgtccacggcgatgtaacggcgggttttgtcgtcgtctacgtggacgtctttggtggtgccgacttcgtagatggtcacggtggaggggcctgcgatagcgcggccgggctcaacaagcacggttggtgcgtcgatgcctagttccgctgccatttttccgactgcggtgagcaggtcggaggcaacttctgcgacgttgagtggttcttcagctgcggtataggcaatgccgtatccgccaccgagatccagttcaggaagggcaacgcccagttcgctgtggatctgtgagtacaggcccaacacgcgttctgctgccagcttgaagccttcggcgtcgaacacctgggaaccaacgtggcagtgcaggccaaccaggttcaggttttctgcgttgttggcggcttttgctgcttcgaatgcggaaccggatgccagggagaatccgaacttctggtcttcgtggctagtggcgatgaactcgtgggtgtgtgcttcgatgcctggctttacgcggatcaacacgtcctgaatcttgccttcaccagcggcaacgtaatccaacagttctagttcctgtgcggagtccagcaccacgtgtcccacaccgttttgaaccaacgcgcgcaggaactctacgcctttgttgttgccgtgcgcggtgatacggctggcggggaaaccagcggccagggcaatgcccagttcgttgatggatgcaatgtccagtgccagcccctcttcatcaacccaacgtgcaatggtcttggtcaggaacgctttagatgcgtagtgcacattgcctggtccaccgaatgcggtagccatgtcgcgacagcgggaacggaaatcgtcctcgtcgactacgaacagtggggttccgtattcttcagcgaggtcaggcagaggcacaccagcgacggtgacaacgccgtcttcttggcgcacggcattgcgtggccatacgtgtgcgggaagttcattgaaattttcaactgtagccattgttacatcttctccggtgcggaaacgccaaccaggtgcagggcgttagcgagggtctggcgggttgctgctgcaagtgccagacgtgctgtgtggattggtgccgtatcctcatcaacctttggaaggatgtggcaggaatcgtagaagcggtggaaagttccagctaattcctcagcatagcgggcaatgcggtgtggttcacgtaggtcagcggcagccttcaccactgctgggaactctccgagtgtgcggatgagatcgccttcgcggtcgtgggtcagtagagataggtctgcgccttcctcggtgacacccaaggtctctgccttgcgcgcgatggagcacagacgagcgtgtccgtactgcacgtagtacacagggttgtcggaggactgggattcccacaggccgagatcgatatccagggaagaatccacggaggaacggatcagggagtaacgcgccgcatcgatgccgattgcttcaacgaggtcatctagggtgaccacggtgcctgcacgcttggacatacgcactgccttgccgtcgcgaagcaggttcaccatctggccaatcaggacttcaacgccttctggcttgtagccaagtgccgccgccgctgccttcaggcgcgcgatgtaaccatggtggtcagcacccaacatgtagatgtttaggttgtgtccgcgggagaacttatcagccacgtacgcgatatcgccagcgatgtaggctgcgtcgccgtcagacttgatcaccacgcggtctttgtcatcgccgaattcggtggaacgcagccaccaagcgccctcgttttcgtacaggttgccgttgtccttcagcacctgcacggccttgtccaccgcaccggactcgaacagggagttctcgtggtagtagacatcgaaatcggtgccgaactcatgcagggaagatttgatgtgctcgaacatcatctccacgccttcagcgcggaaaagctcctgggttgcggcaggctccaaagccaacgcttcaggatgcttttcgacgattgcctccgcaatttccttaatgtattcgccgccataaccgtcttctggcgttggctcgcccttcgccgctgcaagaagggacaaagcgaaacgatcgatctggcgaccgtgatcgttgaagtagtattcgcgggtcactttcgcgccggaagcctccagcacacgacccaaagagtcacccacggcagcccagcgggttccgccaaggtgaataggtccggttgggtttgcagaaacgaactcgaggttcacgtccaagtgggaaaggtgatcggagtttccgaaagtctcgccctgtgccagaatcttggccacaatttcaccctgtgctgctgcagcaaggcgaatgttcaaaaagcctgggccagcaatttcagcagaatcaatggcgtcatctgcagccaatgcctctgccagccaggtagccaaatcccgagggttctgaccgacctttttagccacctgcaatgcaatgttggtggcgtaatcgccgtgctctgggttacgcggacgctccacaactacctgctccggaagaacagaagtatcgagctcgcgggaggtcaaaacctctaccgcggtctctttaatcaatgttgcgagatcagctggtgtcatggggagaaattctagccgaggtacacccgtcgcacagaatacgacccatacttataacaaatccctgcctttttaatgaaaaatcattggtctctgtatatgctttttggggtaactgacccagaaaacaaacgtccttggagccgtaatgccccgttcgacaataaaaagggtagtagcagttcttgccgcctcgactgcgcttagcccctttttggtatcaatgcccactgcagcagcgcaagaaaacatccgctgggaagaatgcccacctcaggtagatattgcctccgctcaatgtggcagcatcgacgtgcccatgcactattctgatccctcacttggcgatatcagcgtgggctttgtcaaggtccctgcccaaggcgaaaagcacggcaccatcttcggtaactccggtggccctggtggcgatgcctatagcttcttcggcagccaatccatgaactggccagaagccatgtaccaaaactacgacctcgttgcagtgcagcctctctagaaagaaacccagaaacccaaaaccgccctccacggcgaaggtgaagggcggggtcaggcgtcgaaaagcgttatgcctctagtgagaggatgtcgtcgagcgtttcgcggcgcagcatgaggcgggagctgccagcgcggacggacacgacggcgggccgtgtgaaggcgttgtagcgggagctcatggcgtagcagtatgcgccggtggctgcgagtgcaaggaagtcgccgctggtgatgtcagatgggtagatttcatcgttgatcaggatatcgccggattcgcagtgggagcccacgatgcgggtgcttactgggtctccttcggcgaagcgggatactacgcgggcgtcgtattcggagccgtagagtgctgggcggatgttgtcggacatgcctccgtccacggcgatgtaacggcgggttttgtcgtcgtctacgtggacgtctttggtggtgccgacttcgtagatggtcacggtggaggggcctgcgatagcgcggccgggctcaacaagcacggttggtgcgtcgatgcctagttccgctgccatttttccgactgcggtgagcaggtcggaggcaacttctgcgacgttgagtggttcttcagctgcggtataggcaatgccgtatccgccaccgagatccagttcaggaagggcaacgcccagttcgctgtggatctgtgagtacaggcccaacacgcgttctgctgccagcttgaagccttcggcgtcgaacacctgggaaccaacgtggcagtgcaggccaaccaggttcaggttttctgcgttgttggcggcttttgctgcttcgaatgcggaaccggatgccagggagaatccgaacttctggtcttcgtggctagtggcgatgaactcgtgggtgtgtgcttcgatgcctggctttacgcggatcaacacgtcctgaatcttgccttcaccagcggcaacgtaatccaacagttctagttcctgtgcggagtccagcaccacgtgtcccacaccgttttgaaccaacgcgcgcaggaactctacgcctttgttgttgccgtgcgcggtgatacggctggcggggaaaccagcggccagggcaatgcccagttcgttgatggatgcaatgtccagtgccagcccctcttcatcaacccaacgtgcaatggtcttggtcaggaacgctttagatgcgtagtgcacattgcctggtccaccgaatgcggtagccatgtcgcgacagcgggaacggaaatcgtcctcgtcgactacgaacagtggggttccgtattcttcagcgaggtcaggcagaggcacaccagcgacggtgacaacgccgtcttcttggcgcacggcattgcgtggccatacgtgtgcgggaagttcattgaaattttcaactgtagccattgttacatcttctccggtgcggaaacgccaaccaggtgcagggcgttagcgagggtctggcgggttgctgctgcaagtgccagacgtgctgtgtggattggtgccgtatcctcatcaacctttggaaggatgtggcaggaatcgtagaagcggtggaaagttccagctaattcctcagcatagcgggcaatgcggtgtggttcacgtaggtcagcggcagccttcaccactgctgggaactctccgagtgtgcggatgagatcgccttcgcggtcgtgggtcagtagagataggtctgcgccttcctcggtgacacccaaggtctctgccttgcgcgcgatggagcacagacgagcgtgtccgtactgcacgtagtacacagggttgtcggaggactgggattcccacaggccgagatcgatatccagggaagaatccacggaggaacggatcagggagtaacgcgccgcatcgatgccgattgcttcaacgaggtcatctagggtgaccacggtgcctgcacgcttggacatacgcactgccttgccgtcgcgaagcaggttcaccatctggccaatcaggacttcaacgccttctggcttgtagccaagtgccgccgccgctgccttcaggcgcgcgatgtaaccatggtggtcagcacccaacatgtagatgtttaggttgtgtccgcgggagaacttatcagccacgtacgcgatatcgccagcgatgtaggctgcgtcgccgtcagacttgatcaccacgcggtctttgtcatcgccgaattcggtggaacgcagccaccaagcgccctcgttttcgtacaggttgccgttgtccttcagcacctgcacggccttgtccaccgcaccggactcgaacagggagttctcgtggtagtagacatcgaaatcggtgccgaactcatgcagggaagatttgatgtgctcgaacatcatctccacgccttcagcgcggaaaagctcctgggttgcggcaggctccaaagccaacgcttcaggatgcttttcgacgattgcctccgcaatttccttaatgtattcgccgccataaccgtcttctggcgttggctcgcccttcgccgctgcaagaagggacaaagcgaaacgatcgatctggcgaccgtgatcgttgaagtagtattcgcgggtcactttcgcgccggaagcctccagcacacgacccaaagagtcacccacggcagcccagcgggttccgccaaggtgaataggtccggttgggtttgcagaaacgaactcgaggttcacgtccaagtgggaaaggtgatcggagtttccgaaagtctcgccctgtgccagaatcttggccacaatttcaccctgtgctgctgcagcaaggcgaatgttcaaaaagcctgggccagcaatttcagcagaatcaatggcgtcatctgcagccaatgcctctgccagccaggtagccaaatcccgagggttctgaccgacctttttagccacctgcaatgcaatgttggtggcgtaatcgccgtgctctgggttacgcggacgctccacaactacctgctccggaagaacagaagtatcgagctcgcgggaggtcaaaacctctaccgcggtctctttaatcaatgttgcgagatcagctggtgtcatggggagaaattctagccgaggtacacccgtcgcacagaatacgacccatacttataacaaatccctgcctttttaatgaaaaatcattggtctctgtatatgctttttggggtaactgacccagaaaacaaacgtccttggagccgtaatgccccgttcgacaataaaaagggtagtagcagttcttgccgcctcgactgcgcttagcccctttttggtatcaatgcccactgcagcagcgcaagaaaacatccgctgggaagaatgcccacctcaggtagatattgcctccgctcaatgtggcagcatcgacgtgcccatgcactattctgatccctcacttggcgatatcagcgtgggctttgtcaaggtccctgcccaaggcgaaaagcacggcaccatcttcggtaactccggtggccctggtggcgatgcctatagcttcttcggcagccaatccatgaactggccagaagccatgtaccaaaactacgacctcgttgcagtgcagcctcggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataa

SEQ ID NO: 25 - DNA, 5027 bp, Corynebacterium glutamicum
tcgagaggcctgacgtcgggcccggtaccacgcgtcatatgactagccaattcagccaaaacccccacgcgatcttccacatccatgtcgaggtggtaacgagtggtggtctcaccgaaatcagcgatcggcaggttagcgtaggtggactcacctggagcacggccaccgtgcaccttgtttcgtgcggcaccaacgacgtcgccaagcacagcagacgcggttggcgcgccacctgcaccgtttccgtagaacatcaggcgaccagctgcttctgcttcaacaaagattgcattaaaggacttgtttaccgacgccagtgggtgggacacaggtaatagagtcgggtgcacgcgagcagaaatagccgactttccttccttgttggtgaacttctcacagatggccaacaacttgatggtgtggcctgcctgctgtgctgcctcaatgtcggcagcgctgatgttgctgataccttcgcagtacacatcatccgcggtaacacggcagtgccgactgttccgaatcctaaaagggcaattccgactgctgagccgggacccttgccggggttaaagcttggggcagatgctgaggtcatgattctccaaaaataatcgcggtcgagcgttgttgtcctattactttgtttcggccaccctacatgtgtccaaaacaaaaagggtacctagccactatagaccaagctgttcatttttgctttaaagccccgaaattcgattcaccttcccagctttttagttttcaacggaaaaactctcaagatttttagtatatttatgaaatctaatatcaacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 26 - DNA, 6986 bp, Corynebacterium glutamicum
ccgcggatcaataatgacgattgacggcattacgtctaacgatatttacatgcttggttatgtttataattctttaactggcccatacaagccgctgaacaaaactggccttgtgttaagaatggatcttgatcctaacgatgtaacctyttacttactcacacttcgctgtacctcaagcgaraaggaaacaatgtcgtgattacaagctatatgacaaacagaggattctacgcagacaaacaatcaacgtttgcgccgagcttcytgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagcagactttgctgccgatccacaaaagtacgacatcccagactctgtcatcgcgttcctgcgcggcgagcttggtaaccctccaggtggctggccagagccactgcgcacccgcgcactggaaggccgctccgaaggcaaggcacctctgacggaagttcctgaggaagagcaggcgcacctcgacgctgatgattccaaggaacgtcgcaatagcctcaaccgcctgctgttcccgaagccaaccgaagagttcctcgagcaccgtcgccgcttcggcaacacctctgcgctggatgatcgtgaattcttctacggcctggtcgaaggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcaccgtgactgttgctgaaggtgatgaggtcaaggctggagatgcagtcgcaatcatcgaggctatgaagatggaagcaacaatcactgcttctgttgacggcaaaatcgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggttgagttagcaaacggcgctctcggtatgattgagctaaacgatgattcacactgaaaaaagtgatgaaaccgctgatgcatctacacagtacagatgaaattgacgcgcgaacgtctttaaatgaacggaaatggt

SEQ ID NO: 27 - DNA, 5622 bp, Corynebacterium glutamicum
tcgagaggcctgacgtcgggcccggtaccacgcgtcatatgactagccaattcagccaaaacccccacgcgatcttccacatccatgtcgaggtggtaacgagtggtggtctcaccgaaatcagcgatcggcaggttagcgtaggtggactcacctggagcacggccaccgtgcaccttgtttcgtgcggcaccaacgacgtcgccaagcacagcagacgcggttggcgcgccacctgcaccgtttccgtagaacatcaggcgaccagctgcttctgcttcaacaaagattgcattaaaggacttgtttaccgacgccagtgggtgggacacaggtaatagagtcgggtgcacgcgagcagaaatagccgactttccttccttgttggtgaacttctcacagatggccaacaacttgatggtgtggcctgcctgctgtgctgcctcaatgtcggcagcgctgatgttgctgataccttcgcagtacacatcatccgcggtaacacgggtgtggaaagcgatggatgccaaaattgcagccttggatgcggcgtcatggccttcgacgtctgcagttggatcagcttcggcgtaacccaaacgagttgcctcagccaaagaatctgcatagtcagcgccggtggaatccatggcgtccaagatgaagttggtggtgccgttaacgatgcccatcacagactggatctgatcgccagccagggagcgacgcagtgggccaaccactggaattgcgcctgcaacagcagcctcgaagtacaggtcaacgtttgcggcttccgctgcatcagcaagctcagcagagtgagctgcaacaagagccttattggcggtaacaacagacttgccggccttcagagctgcgagaactacctcacgtgggtactcaatgccgccgataacctcaacgacgatgtcaacatcctcgcgctcgatgagtgcaaaagcgtcctcagtgagcagctcaggtgcaacgccttcacgtggctttgagatatcagaagcagcaatgccacgaacctccagtgggccaccaatgcggtgcgcaagttcatcaccgtactcggtcatcagacgcatcacctcagtgccgactgttccgaatcctaaaagggcaattccgactgctgagccgggacccttgccggggttaaagcttggggcagatgctgaggtcatgattctccaaaaataatcgcggtcgagcgttgttgtcctattactttgtttcggccaccctacatgtgtccaaaacaaaaagggtacctagccactatagaccaagctgttcatttttgctttaaagccccgaaattcgattcaccttcccagctttttagttttcaacggaaaaactctcaagatttttagtatatttatgaaatctaatatcaacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 28 - DNA, 8900 bp, Corynebacterium glutamicum
ccgcggatcaataatgacgattgacggcattacgtctaacgatatttacatgcttggttatgtttataattctttaactggcccatacaagccgctgaacaaaactggccttgtgttaagaatggatcttgatcctaacgatgtaacctyttacttactcacacttcgctgtacctcaagcgaraaggaaacaatgtcgtgattacaagctatatgacaaacagaggattctacgcagacaaacaatcaacgtttgcgccgagcttcytgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgtcttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggattcattgccgatcactcgcacctccttcaggctccacctgctgatgatgagcagggacgcatcctggattacttggcagatgtcaccgtgaacaagcctcatggtgtgcgtccaaaggatgttgcagctcctatcgataagctgcctaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccagctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacatcttggccattaaggatatggctggtctgcttcgcccagctgcggtaaccaagctggtcaccgcactgcgccgtgaattcgatctgccagtgcacgtgcacacccacgacactgcgggtggccagctggcaacctactttgctgcagctcaagctggtgcagatgctgttgacggtgcttccgcaccactgtctggcaccacctcccagccatccctgtctgccattgttgctgcattcgcgcacacccgtcgcgataccggtttgagcctcgaggctgtttctgacctcgagccgtattgggaagcagtgcgcggactgtacctgccatttgagtctggaaccccaggcccaaccggtcgcgtctaccgccacgaaatcccaggcggacagttgtccaacctgcgtgcacaggccaccgcactgggccttgcggatcgtttcgaactcatcgaagacaactacgcagccgttaatgagatgctgggacgcccaaccaaggtcaccccatcctccaaggttgttggcgacctcgcactccacctcgttggtgcgggtgtggatccagcagactttgctgccgatccacaaaagtacgacatcccagactctgtcatcgcgttcctgcgcggcgagcttggtaaccctccaggtggctggccagagccactgcgcacccgcgcactggaaggccgctccgaaggcaaggcacctctgacggaagttcctgaggaagagcaggcgcacctcgacgctgatgattccaaggaacgtcgcaatagcctcaaccgcctgctgttcccgaagccaaccgaagagttcctcgagcaccgtcgccgcttcggcaacacctctgcgctggatgatcgtgaattcttctacggcctggtcgaaggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcaccgtgactgttgctgaaggtgatgaggtcaaggctggagatgcagtcgcaatcatcgaggctatgaagatggaagcaacaatcactgcttctgttgacggcaaaatcgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggttgagttagcaaacggcgctctcggtatgattgagctaaacgatgattcacactgaaaaaagtgatgaaaccgctgatgcatctacacagtacagatgaaattgacgcgcgaacgtctttaaatgaacggaaatggt

SEQ ID NO: 29 - DNA, 5516 bp, Corynebacterium glutamicum
tcgaggctgtggcagtgaccaaccgtctgacagttgctgatctggctgataccttcgcggtgtacccatcattgtcaggttcgattactgaagcagcacgtcagctggttcaacatgatgatctaggctaatttttctgagtcttagattttgagaaaacccaggattgctttgtgcactcctgggttttcactttgttaagcagttttggggaaaagtgcaaagtttgcaaagtttagaaatattttaagaggtaagatgtctgcaggtggaagcgtttaaatgcgttaaacttggccaaatgtggcaacctttgcaaggtgaaaaactggggcggggttagatcctggggggtttatttcattcactttggcttgaagtcgtgcaggtcaggggagtgttgcccgaaaacattgagaggaaaacaaaaaccgatgtttgattgggggaatcgggggttacgatactaggacgcagtgactgctatcacccttggcggtctcttgttgaaaggaataattactctatagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccgtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgacgcgtcatatgactagttcggacctagggatatcgtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 30 - DNA, 7043 bp, Corynebacterium glutamicum
cgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgaaatttgctgggtggtggtatccggaagttcaaagatcattttttgcccctaaattatggcctgcgccaggtgtgaccgttgcgggaaagcatttcatcagcgctctttggaccccacgtacccgctgggtaatcctctggttctccatcggcatcccatgcttcaagaattggatccagaatcttccagctcagttccacttcctcgttggtagggaagaggctggattcatctaacagcgcatccaaaatgaggcgctcgtatgcttcaggtgattcttcagtgaaggattctgagtaggagaagtccatgttgacgtcacggacttccatggcagaacctggaaccttggaaccgaagcggatgagcacaccttcatcaggctgcacgcgaatcacgatggcgttttggccaagggatacagtcatgtcgccgtcgaaaggctggtgtggtgcgtctttaaacaccacggcaatctcagtaacacggcgaccaagacgcttaccggtgcgcaggtagaacggcacaccagcccagcgacgagacgtgatctctaaggtacaagccgcaaaagtctcagtggtggactcagggttgaagccatcttcttcgcgaagtcccttgactaactcagagccctgccaaccggcagcgtactgaccacgagcggaggttttatccaatgggtagcacggctttgtcgcagagagcaccttgatcttttctgccaattccgtacgagcaccagcacttgcggcatcgcgtacgtatttttcaaagtcttctttggaccattcgcggcggccgtaacctaccaacgagaatcctgggggcagcaatccgcggtttgctagatcataaatggcggggagcagcttctttcgagccaagtcgccagtgacaccgaagatcaccatgccggaagggccagcgatgcgggggagtcgtttatcctgcgggtcgcgcagtgggtttgtccagctggagggggtcgtgtttgtgctcacgatggtagtgtcacgatcctttctttaatgaaagatgtgtaacggccacataagatcgaactaattcgatttcatgtcgccgttactgatgcagcgtgctgattctacttcagacgagcttccatggactcaagcagttcgctccaagaagcaacgaacttgtccacaccctcggtctccaggacctggaagacatctgccaagtcaacgcccagagcctcaagctgggagaacacagcgtcagcttctgccgcggagttggacagggtgtcaccgtgcaggttgccctgctccagaaccgcgtcgatggtgccttctggcatggtgttgacggtgtttggaccagccagctcggaaacgtaaagagttgcagcgtacgcagggttcttcacgccggtggatgcccacagtgggcgctgagtgttggcaccttcaggcagctcggcggcgtcgaaaagctccttgtacacaggaattccggcg

SEQ ID NO: 31 - DNA, 7632 bp, Corynebacterium glutamicum
cgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgaaatttgctgggtggtggtatccggaagttcaaagatcattttttgcccctaaattatggcctgcgccaggtgtgaccgttgcgggaaagcatttcatcagcgctctttggaccccacgtacccgctgggtaatcctctggttctccatcggcatcccatgcttcaagaattggatccagaatcttccagctcagttccacttcctcgttggtagggaagaggctggattcatctaacagcgcatccaaaatgaggcgctcgtatgcttcaggtgattcttcagtgaaggattctgagtaggagaagtccatgttgacgtcacggacttccatggcagaacctggaaccttggaaccgaagcggatgagcacaccttcatcaggctgcacgcgaatcacgatggcgttttggccaagggatacagtcatgtcgccgtcgaaaggctggtgtggtgcgtctttaaacaccacggcaatctcagtaacacggcgaccaagacgcttaccggtgcgcaggtagaacggcacaccagcccagcgacgagacgtgatctctaaggtacaagccgcaaaagtctcagtggtggactcagggttgaagccatcttcttcgcgaagtcccttgactaactcagagccctgccaaccggcagcgtactgaccacgagcggaggttttatccaatgggtagcacggctttgtcgcagagagcaccttgatcttttctgcctgcagctgcgctggcacgaaagaaattggttcttccatggcaaccagagccaagagctggatcaggtggttctggatgacgtcgcgggctgcgccgatgccgtcgtagtaaccagcacgtccacccaagccaatatcttcagtcatggtgatctggacgtggtcaacgtagttggagttccacagtggctcaaacagctggttagcaaaacgcagagccaggatgttttgaactgtttccttgcccaaatagtggtcgatgcggaacacagaagattctgggaagactgcgttgaccagctggttgagctcgtgtgcggattcgaggttgtggccgaaaggcttctcgatgatcacgcggcgccatgcttcttcggtggattcagccatgccggaacgctccagctggtggcagaccgctgtgaaggaatctggtggaatggacaggtagtaagcccagttgccggcggtgccgcgggttttgtcgatgcgcttgagtgttgcagcgaggttgtcgaaagctgcatcatcatcaaagttgccgcgaacaaattccataccctcggcgaggcgctcccaaacattttcacggaattccgtacgagcaccagcacttgcggcatcgcgtacgtatttttcaaagtcttctttggaccattcgcggcggccgtaacctaccaacgagaatcctgggggcagcaatccgcggtttgctagatcataaatggcggggagcagcttctttcgagccaagtcgccagtgacaccgaagatcaccatgccggaagggccagcgatgcgggggagtcgtttatcctgcgggtcgcgcagtgggtttgtccagctggagggggtcgtgtttgtgctcacgatggtagtgtcacgatcctttctttaatgaaagatgtgtaacggccacataagatcgaactaattcgatttcatgtcgccgttactgatgcagcgtgctgattctacttcagacgagcttccatggactcaagcagttcgctccaagaagcaacgaacttgtccacaccctcggtctccaggacctggaagacatctgccaagtcaacgcccagagcctcaagctgggagaacacagcgtcagcttctgccgcggagttggacagggtgtcaccgtgcaggttgccctgctccagaaccgcgtcgatggtgccttctggcatggtgttgacggtgtttggaccagccagctcggaaacgtaaagagttgcagcgtacgcagggttcttcacgccggtggatgcccacagtgggcgctgagtgttggcaccttcaggcagctcggcggcgtcgaaaagctccttgtacacaggaattccgg

SEQ ID NO: 32 - DNA, 7207 bp, Corynebacterium glutamicum
aaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgacggcacctgcaactaacttttgggacctatgtgcgtgactgctttcaaaccttcacaaaacatgtctctaaacatgcaggtcagggcgcgattttgcggtttgaaaaaagtcacgtatattcttatccagtcgccaagagcgacgagctcccatcgtctaggggcctaggacactgccctttcacggcagcgacacgggttcgaatcccgttgggagtactacttccccaaaggaagtagcagtgaaatgctgcacatgaggccctgtggcgcagttggttagcgcgccgccctgtcacggcggaggtcgcgggttcaagtcccgtcagggtcgcagcaatgtgataacagaggttgtactttttcttcaagaaattgtacagcctctgttttttattttctggtgtgtttttggggtctcaggtttcgggtttcttaagagcgggggtttcttaagttcgacttcttgcttacgacttcacggtagcgacttcttatgctcaacttctcgtggtcgatttcagtcggattttcctggatttcctagtgaagtcgcctttgagaagtcggtgttaagaagtcgaactctaggggctgtcaggttaagcctcacggatgtcatgttttgaccggctagttgccggaaaacctgcggaaaagttcccaaggggtgggggctgagcaccacggatcatcgtggctgcagctggtgaacgccatggtgggcagcgccccgcgaacttcctagacattggtggcggagcatcagctgaatcgatggctgctggtctcgatgtgatccttggggatagccaggtacgcagtgtgtttgtgaatgtgtttggtggcatcaccgcgtgtgatgtggtggcaaagggaatcgttggagctttggatgtgctcggcgatcaagcaacgaagcctcttgtggtgcgccttgatggcaacaacgtggtggaaggcagacgaatcctcgcggaatataaccaccctttggtcaccgttgtggagggtatggatgcagcggctgatcacgctgcccatttggccaatcttgcccagcacggccagttcgcaaccgctaattagttaaggagcacctgtttaatcatgtctatttttctcaattcagattcccgcatcatcattcagggcattaccggttcggaaggttcagagcatgcgcgtcgaattttagcctctggtgcgaagctcgtgggtggcaccaacccccgcaaagctgggcaaaccattttgatcaatgacactgagttgcctgtatttggcactgttaaggaagcaatggaggaaacgggtgcggatgtcaccgtaattttcgttcctccagcctttgccaaagctgcgatcattgaagctatcgacgctcacatcccactgtgcgtgattattactgagggcatcccagtgcgtgacgcttctgaggcgtgggcttatgccaagaaggtgggacacacccgcatcattggccctaactgcccaggcattattactcccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataa

SEQ ID NO: 33 - DNA, 8149 bp, Corynebacterium glutamicum
aaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgacggcacctgcaactaacttttgggacctatgtgcgtgactgctttcaaaccttcacaaaacatgtctctaaacatgcaggtcagggcgcgattttgcggtttgaaaaaagtcacgtatattcttatccagtcgccaagagcgacgagctcccatcgtctaggggcctaggacactgccctttcacggcagcgacacgggttcgaatcccgttgggagtactacttccccaaaggaagtagcagtgaaatgctgcacatgaggccctgtggcgcagttggttagcgcgccgccctgtcacggcggaggtcgcgggttcaagtcccgtcagggtcgcagcaatgtgataacagaggttgtactttttcttcaagaaattgtacagcctctgttttttattttctggtgtgtttttggggtctcaggtttcgggtttcttaagagcgggggtttcttaagttcgacttcttgcttacgacttcacggtagcgacttcttatgctcaacttctcgtggtcgatttcagtcggattttcctggatttcctagtgaagtcgcctttgagaagtcggtgttaagaagtcgaactctaggggctgtcaggttaagcctcacggatgtcatgttttgaccggctagttgccggaaaacctgcggaaaagttcccaaggggtgggggctgagcaccacggatccaattttgttgcaatttgcaaagtttacagtgttagacttcacaatacgatcatattggtgagttgaaacacttacttttacgggaagactttgttaaagacgcagaaggctctaagcatgggccggaaatggaattggcagtggatctttttgaataccaagcacgggacctctttgaaacccatggtgtgccagtgttgaagggaattgtggcatcaacaccagaggcggcgaggaaagcggctgaggaaatcggcggactgaccgtcgtcaaggctcaggtcaaggtgggcggacgtggcaaggcgggtggcgtccgtgtggcaccgacgtcggctcaggcttttgatgctgcggatgcgattctcggcatggatatcaaaggacacactgttaatcaggtgatggtggcgcagggcgctgacattgctgaggaatactatttctccattttgttggatcgcgcgaatcgttcgtatctggctatgtgctctgttgaaggtggcatggagatcgagatcctggcgaaggaaaagcctgaagctttggcaaaggtggaagtggatcccctcactggtattgatgaggacaaagcgcgggagattgtcactgctgctggctttgaaactgaggtggcagagaaagtcattccggtgctgatcaagatctggcaggtgtattacgaagaggaagcaacactcgttgaggtgaacccgttggtgctcacggatgacggcgatgtgattgcgcttgatggcaagatcacgctggatgataacgctgatttccgccatgataaccgtggtgcgttggctgaatctgccggtggcttggacattttggaactgaaggccaagaagaatgatctgaactacgtgaaacttgatggctctgtgggcatcattggcaatggtgcaggtttggtgatgtccacgttggatatcgtggctgcagctggtgaacgccatggtgggcagcgccccgcgaacttcctagacattggtggcggagcatcagctgaatcgatggctgctggtctcgatgtgatccttggggatagccaggtacgcagtgtgtttgtgaatgtgtttggtggcatcaccgcgtgtgatgtggtggcaaagggaatcgttggagctttggatgtgctcggcgatcaagcaacgaagcctcttgtggtgcgccttgatggcaacaacgtggtggaaggcagacgaatcctcgcggaatataaccaccctttggtcaccgttgtggagggtatggatgcagcggctgatcacgctgcccatttggccaatcttgcccagcacggccagttcgcaaccgctaattagttaaggagcacctgtttaatcatgtctatttttctcaattcagattcccgcatcatcattcagggcattaccggttcggaaggttcagagcatgcgcgtcgaattttagcctctggtgcgaagctcgtgggtggcaccaacccccgcaaagctgggcaaaccattttgatcaatgacactgagttgcctgtatttggcactgttaaggaagcaatggaggaaacgggtgcggatgtcaccgtaattttcgttcctccagcctttgccaaagctgcgatcattgaagctatcgacgctcacatcccactgtgcgtgattattactgagggcatcccagtgcgtgacgcttctgaggcgtgggcttatgccaagaaggtgggacacacccgcatcattggccctaactgcccaggcattattactcccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataa

SEQ ID NO: 34 - DNA, 5487 bp, Corynebacterium glutamicum
tcgaggacgatgacaaccgatacagcgattttcttccacgcactccactgggcaacactgcgcagagcgtcatggaaaagtgagctaaagaagtgaaagaccgtggaaattggtgtgtttaggcgtcctgacatgtccgcaggattctcctgggtctcacctttgtcatgcatagaaaacaccatacagttgcacccccgtgatcaaaatattccgccattttgaatgtattgaaacaaagttctcgttatcgtagtagaagctagtgccgatccctgctcagctgttcgcagaagggtattaagttcggcaattttcgtaggtaaacacaggtgaaggctttacaagcttgtgaactccctacacaaaagcaatccaatagctatccataagcaagagaaagtaagtctacgttgactgatctcacaaagactgcggtgcccgaggaactttcagagaacctcgaaacttggtacaaggctgtggccggtgttttcgcgcgcacacagaaaaaagacatcggcgacattgccgtagatgtgtggaagaaactcatcgtcacgcattgtagtggtgtgtggaacggaccaagagtatgccgaaacgggggagggagccgtcgaaaagctccgcgaagcgggcgttgagcgcatcctgcttgctggcgcgccgaagagctttgagggcagcgcgcatgcgcccgatggttatttgaacatgacaattgatgccgcggcgacgctggctgacctgctagatgctttgggagcttaaatcatgacgtcgatccctaatttttcagacatcccattgactgctgagacacgtgcatcggagtcacacaacgttgacgccggcaaggtgtggaacactcccgaaggcattgatgtcaagcgcgtattcacgcaggctgaccgcgacgaggcgcaagcggcgggacatccggtggattctttgccaggtcaaaagccatttatgcgcgggccgtacccaactatgtacaccaatcagccgtggacgattcgccagtacgcaggcttttcaaccgccgcggaatccaatgcgttttatcggaggaaccttgctgcgggtcaaaaaggtttgtcggttgcgttcgatctagcgacccaccgcggttatgactcggataatgagcgcgtggtcggcgatgtgggtatggccggcgtggcgattgattcgattttggatatgcgtcagctgtttgatggcattgatttgtccagcgtgtcggtgtcgatgaccatgaatggtctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 35 - DNA, 6977 bp, Corynebacterium glutamicum
tcgaggacgatgacaaccgatacagcgattttcttccacgcactccactgggcaacactgcgcagagcgtcatggaaaagtgagctaaagaagtgaaagaccgtggaaattggtgtgtttaggcgtcctgacatgtccgcaggattctcctgggtctcacctttgtcatgcatagaaaacaccatacagttgcacccccgtgatcaaaatattccgccattttgaatgtattgaaacaaagttttcgttatcgtagtagaagctagtgccgatccctgctcagctgttcgcagaagggtattaagttcggcaattttcgtaggtaaacacaggtgaaggctttacaagcttgtgaactccctacacaaaagcaatccaatagctatccataagcaagagaaagtaagtctacgttgactgatctcacaaagactgcggtgcccgaggaactttcagagaacctcgaaacttggtacaaggctgtggccggtgttttcgcgcgcacacagaaaaaagacatcggcgacattgccgtagatgtgtggaagaaactcatcgtcactacaccggatggtgttgatatcaatccgctgtacaccagagcagatgagtcccagaggaaattcactgaggtttctggtgagtttcccttcactaggggaaccactgttgatggtgaacgcgttggttggggtgttactgagactttcggacatgacagcccgaagaatatcaacgctgcggtgctgaatgctctgaattctggcaccaccacattgggttttgagttctctgaggaattcacggcagctgatcttaaagttgctctcgaaggcgtgtatctcaacatggctccgttgctgattcatgcgggtggatccacgtcagaggttgcagcggcgttgtatacgttggcggaggaagccggaacgttttttgctgcgttgaccttgggttctcgtcctttgacggcgcaggttgatggttcgcacagtgacaccattgaagaagcagttcagttggcagtgaatgcttccaagcgtgcgaatgtgcgcgctatcttggtggatggttccagtttttccaaccagggcgcgtcggatgctcaagaaattggtctaagtatcgccgccggtgtggattatgtccgtcgcttggtcgatgcaggcctttccacggaagctgcacttaagcaggtggcgttccgttttgcggtcaccgatgaacagttcgcgcagatttctaagctgcgtgtggctcgacgtctgtgggccagggtgtgtgaggtgcttggttttccagagctggccgtagcaccacagcatgcggtgactgcacgagcgatgtttagccagcgtgatccgtgggtgaatatgctgcgcagtactgttgcagctttcgctgcaggcgtcggtggagcaaccgatgtggaggttcgtacttttgatgatgcgatcccagatggagttcctggagtgtcgaggaatttcgctcaccgcatcgcgcgcaatactaatttgttgttgctagaagagtcacatcttggtcacgtggttgatcctgctggtggatcatatttcgtggagagcttcaccgatgatctagcggagaaggcgtgggctgtgttcagtggcatcgaagctgagggcgaatacagtgcagtttgtgcatccggcacggtgactgccatgcttgatcagacgtgggagcagactcgcgctgatgtggcgtcgagaaagaagaagctcactggaattaatgagttcccgaacttggcggagtctccgctgccagctgatcgtcgggtagaacctgcaggtgtgcgtcgatgggcagcggattttgaagcgctgcgcaatcgttcggatgctttcttggaaaagaacggcgcgaggccacagatcacgatgattcctctgggaccgttgtccaagcacaatattcgcactggttttacttccaacctgttggcttccggtggcattgaagcaatcaacccgggtcaacttgttcccggcactgacgcttttgcagaagctgcacaggccgcaggcattgtagtggtgtgtggaacggaccaagagtatgccgaaacgggggagggagccgtcgaaaagctccgcgaagcgggcgttgagcgcatcctgcttgctggcgcgccgaagagctttgagggcagcgcgcatgcgcccgatggttatttgaacatgacaattgatgccgcggcgacgctggctgacctgctagatgctttgggagcttaaatcatgacgtcgatccctaatttttcagacatcccattgactgctgagacacgtgcatcggagtcacacaacgttgacgccggcaaggtgtggaacactcccgaaggcattgatgtcaagcgcgtattcacgcaggctgaccgcgacgaggcgcaagcggcgggacatccggtggattctttgccaggtcaaaagccatttatgcgcgggccgtacccaactatgtacaccaatcagccgtggacgattcgccagtacgcaggcttttcaaccgccgcggaatccaatgcgttttatcggaggaaccttgctgcgggtcaaaaaggtttgtcggttgcgttcgatctagcgacccaccgcggttatgactcggataatgagcgcgtggtcggcgatgtgggtatggccggcgtggcgattgattcgattttggatatgcgtcagctgtttgatggcattgatttgtccagcgtgtcggtgtcgatgaccatgaatggtctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 36 - DNA, 5685 bp, Corynebacterium glutamicum
cgcgtttgcccaaatagtggtcgatgcggaacacagaagattctgggaagactgcgttgaccagctggttgagctcgtgtgcggattcgaggttgtggccgaaaggcttctcgatgatcacgcggcgccatgcttcttcggtggattcagccatgccggaacgctccagctggtggcagaccgctgtgaaggaatctggtggaatggacaggtagtaagcccagttgccggcggtgccgcgggttttgtcgatgcgcttgagtgttgcagcgaggttgtcgaaagctgcatcatcatcaaagttgccgcgaacaaattccataccctcggcgaggcgctcccaaacattttcacggaattccgtacgagcaccagcacttgcggcatcgcgtacgtatttttcaaagtcttctttggaccattcgcggcggccgtaacctaccaacgagaatcctgggggcagcaatccgcggtttgctagatcataaatggcggggagcagcttctttcgagccaagtcgccagtgacaccgaagatcaccatgccggaagggccagcgatgcgggggagtcgtttatcctgcgggtcgcgcagtgggtttgtccagctggagggggtcgtgtttgtgctcacgggtaaaaaatcctttcgtaggtttccgcaccgagcatatacatcttttgaaaatccgtcagatggcgcttcgcaaaagtacttggtgcgacacctcccaatgataggcctttttgttgatattgcaacgaaatttttcagccgacctatttatcgggtagcgggtcacaagcccggaataattggcagctagatggtagtgtcacgatcctttctttaatgaaagatgtgtaacggccacataagatcgaactaattcgatttcatgtcgccgttactgatgcagcgtgctgattctacttcagacgagcttccatggactcaagcagttcgctccaagaagcaacgaacttgtccacaccctcggtctccaggacctggaagacatctgccaagtcaacgcccagagcctcaagctgggagaacacagcgtcagcttctgccgcggagttggacagggtgtcaccgtgcaggttgccctgctccagaaccgcgtcgatggtgccttctggcatggtgttgacggtgtttggaccagccagctcggaaacgtaaagagttgcagcgtacgcagggttcttcacgccggtggatgcccacagtgggcgctgagtgttggcaccttcaggcagctcggcggcgtcgaaaagctccttgtacacagcgtaagcgcgctgagcgttggcaacgcctgccttgccgcgcagagccaaagcctcatcggatccgattgcctcgaggcgcttgtcgatctcaacgtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

SEQ ID NO: 37 - DNA, 7990 bp, Escherichia coli
catcataaagcttccggaagcgatggcggcatcgttgaaagcgcaactgctggcggatctggccgatctcgacgtgttaagcactgaagatttaaaaaatcgtcgttatcagcgcctgatgagctacggttacgcgtaattcgcaaaagttctgaaaaagggtcacttcggtggcccttttttatcgccacggtttgagcaggctatgattaaggaaggattttccaggaggaacacatgaacatcattgccattatgggaccgcatggcgtcttttataaagatgagcccatcaaagaactggagtcggcgctggtggcgcaaggctttcagattatctggccacaaaacagcgttgatttgctgaaatttatcgagcataaccctcgaatttgcggcgtgatttttgactgggatgagtacagtctcgatttatgtagcgatatcaatcagcttaatgaatatctcccgctttatgccttcatcaacacccactcgacgatggatgtcagcgtgcaggatatgcggatggcgctctggttttttgaatatgcgctggggcaggcggaagatatcgccattcgtatgcgtcagtacaccgacgaatatcttgataacattacaccgccgttcacgaaagccttgtttacctacgtcaaagagcggaagtacaccttttgtacgccggggcatatgggcggcaccgcatatcaaaaaagcccggttggctgtctgttttatgattttttcggcgggaatactcttaaggctgatgtctctatttcggtcaccgagcttggttcgttgctcgaccacaccgggccacacctggaagcggaagagtacatcgcgcggacttttggcgcggaacagagttatatcgttaccaacggaacatcgacgtcgaacaaaattgtgggtatgtacgccgcgccatccggcagtacgctgttgatcgaccgcaattgtcataaatcgctggcgcatctgttgatgatgaacgatgtagtgccagtctggctgaaaccgacgcgtaatgcgttggggattcttggtgggatcccgcgccgtgaatttactcgcgacagcatcgaagagaaagtcgctgctaccacgcaagcacaatggccggttcatgcggtgatcaccaactccacctatgatggcttgctctacaacaccgactggatcaaacagacgctggatgtcccgtcgattcacttcgattctgcctgggtgccgtacacccattttcatccgatctaccagggtaaaagtggtatgagcggcgagcgtgttgcgggaaaagtgatcttcgaaacgcaatcgacccacaaaatgctggcggcgttatcgcaggcttcgctgatccacattaaaggcgagtatgacgaagaggcctttaacgaagcctttatgatgcataccaccacctcgcccagttatcccattgttgcttcggttgagacggcggcggcgatgctgcgtggtaatccgggcaaacggctgattaaccgttcagtagaacgagctctgcattttcgcaaagaggtccagcggctgcgggaagagtctgacggttggtttttcgatatctggcaaccgccgcaggtggatgaagccgaatgctggcccgttgcgcctggcgaacagtggcacggctttaacgatgcggatgccgatcatatgtttctcgatccggttaaagtcactattttgacaccggggatggacgagcagggcaatatgagcgaggaggggatcccggcggcgctggtagcaaaattcctcgacgaacgtgggatcgtagtagagaaaaccggcccttataacctgctgtttctctttagtattggcatcgataaaaccaaagcaatgggattattgcgtgggttgacggaattcaaacgctcttacgatctcaacctgcggatcaaaaatatgctacccgatctctatgcagaagatcccgatttctaccgcaatatgcgtattcaggatctggcacaagggatccataagctgattcgtaaacacgatcttcccggtttgatgttgcgggcattcgatactttgccggagatgatcatgacgccacatcaggcatggcaacgacaaattaaaggcgaagtagaaaccattgcgctggaacaactggtcggtagagtatcggcaaatatgatcctgccttatccaccgggcgtaccgctgttgatgcctggagaaatgctgaccaaagagagccgcacagtactcgattttctactgatgctttgttccgtcgggcaacattaccccggttttgaaacggatattcacggcgcgaaacaggacgaagacggcgtttaccgcgtacgagtcctaaaaatggcgggataacttgccagagcggcttccgggcgagtaacgtgctgttaacaaataaaggagacgttatgctgggtttaaaacaggttcaccatattgcgattattgcgacggattatgcggtgagcaaagctttgcccgtaaagctagtccaaaccggtgaacttccaggcgagggagacatctttaacattgaacgcttgactggaattgctggagaggaatttgctcgtttcaaagaccctcttgcgccaaatctggcagcccgacgagagggggtcgagccaatacagtttgatcagattatctcgtggcttcgtggttttgacgacccagatcgcatcattgtggtggagggcgctggtggcctgctggtcagattaggggaagatttcaccctggcagatgttgcctccgctttgaatgcacccttagtgattgtgacaagcaccggattgggaagcctcaacgctgctgaattaagcgttgaggcagcaaaccgccgaggactcacagtgttgggagtcctcggcggttcgatccctcaaaatcctgatctagctacgatgcttaatctcgaagaatttgagagagtcaccggcgtgcccttttggggagctttgccggaagggttgtcacgggtggaggggttcgtcgaaaagcaatcttttccggcccttgatgcctttaagaaaccgccggcaaggtgatcgtgaacaccgtgccttcgccttgcacgctgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtaccacgcgtccagacatcatgtgtgtgggcaaggccctcaccggtggattcatgtccttcgccgctactttatgcacggacaaggtggctcaattaatcagcaccccaaatggcggaggtgcgctgatgcacggccccacttttatggctaatcctctggcctgtgcggtttcgcatgcttcattagaaatcattgagaccggcatgtggcagaaacaggtaaaaagaatcgaagccgaacttatcgcaggcctttccccacttcaacaccttccaggggttgccgatgtccgggttctcggcgcgattggtgtcatcgaaatggaacaaaatgtcaatgtcgaagaagctactcaggctgcattagatcacggtgtgtggatccgcccctttggacgcttgctctatgtcatgcctccatatatcaccacgtcagagcagtgcgcacagatctgcactgcgcttcatgctgcagttaaagggaaataaaccatgccatttttatttgtcagcggtaccggaactggggttgggaaaaccttctccacagccgttttggttcgatacttagccgatcaaggacacgatgttcgaatt

SEQ ID NO: 38 - DNA, 5413 bp, Corynebacterium glutamicum
tcgaggcattgtatactgcgaccactacagctgttttgactgctttgttttgggaaatccagttatcttatttcttttttgacgcctaccattgttccaatatgtctttgcgcggacgcgattgtgtctcacgccaaaaatctaacctgatctgacacggcaatgaatcccaactgcgtcctgaatttcgatattcccgagttccaacgctgctcggatgaccgagacccgcgtactccctccaaccttcagaacgcgaatacccacgttcgcgatacttgccaggagctaactcccaggaatacagctgttcgccaggaaagaaaaaagcatcacgatcacctgatgattcgacaccaaaataggctggttcagtcccgaagaactcaattccctgagctgtaatgaaaggacattggtatggggttgcctctgacatcgtttgagctctccatccttttcatttcgctattctggtatccatgagtcccaccgttttgcctgctacacaagctgacttccctaagatcgtcgatgttctggttgaagcgcttcttaatcacggaatcgctcgcgcgggtgatgaagctatctatttggaggcgacgccgactcgtgcggctcaactatataaccgtctgggattcgtgcccttgggttatatccccctcagatgatgatggcactcctgaactggcgatgtggaaaccgccagcgatgccaactgtttaaccctgaaggcgatttaaggggtcgaggatctcagagtaggccacggtgtgggtgagaccttttcaaggtcgcagaagcgattctggacggtctcaattttgggactcatgggtcagctgtgattcctgccaattcactgatcaagagctcaccacaagcaaaccccagaatccaaaatcgaatggatttttgcataatagcgacccggaagcgcaaaaatctaccacagactgcaagttttcagcattctgtggtagattttcgcatatttgttcgcccaccatccaaatggtgccttattaggctttgtcgaacgcctccaggatgttttgggcagccaacgtcggggtggtggaacctttgcggagggctcgttccatgtcttggctcactacactagttcggacctagggatatcgtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 39 - DNA, 5305 bp, Corynebacterium glutamicum
gatcctctagaagtgagctgtgttgggagtatttcgacaatgagtggggcagacctccggtggatctcaatgctctttttgagatcttgacgttggtagttttccaagtgggtgtcacctggcatgctgtgctgtccaaacgggaagggttccgtcaagcattcgcccaattcgatgttgcaaaagtagccgccttcaatgaggacgacgtggaacgcctacttgatgatctacagatttttagaaaccgaagaaaaatcaacgctgccatcaccaatgccaaagcgttgctggagttaaacgatgaaacaggcacctttgactcaattattgccgaccactcaactgacgccacagccatggtgaagcatctcaaagccttgggctttacccatatcggactgacctccttgagcatcctccagcaggccattggggtcacagagctgaaggctgcctaagatataactccgggacgtcactagacttaggcactatgcaacctgaagaagtgcacatcaaggacgagaccatcaagttaggtcagttcatcaaattggccaaccttgtcgaatcaggcggagcggccaaagatgccatcgctaacggtgatgtcaccgtcaatggtgaagtggatacccgaaggggtaagacacttcgcgatggcgatgtggtgtgcatcggcgaggtatgcgcgcaggtgtctactggtgacgcagccgacgacgattattttgacgaagccaccgcaaacgatgacttcgatcccgaaaagtggaggaacatgtaatgccagcctttgaggcaatgccaggaatgccgtattggatcgacctgtccacctcggacattgcaaaatctgcacacttctacgaaaacgttctcggctgggaaattgaagaagtcaacgatggctaccgcatggctcgtctgcagggactacccgtggcagggctgatcgatcagcgcggtgaatcaagcattctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtaccacgcgtcatatgactagttcggacctagggatatcgtcgacatcgatgctcttctgcgttaattaacaattgg

SEQ ID NO: 40 - DNA, 8198 bp, Escherichia coli
tcgagcaacgctgtatataacctgcgtacggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatggagttcaatatgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataacggctaactgtgcagtccgttggcccggttatcggtagcgataccgggcatttttttaaggaacgatcgatatgtatatcgggatagatcttggcacctcgggcgtaaaagttattttgctcaacgagcagggtgaggtggttgctgcgcaaacggaaaagctgaccgtttcgcgcccgcatccactctggtcggaacaagacccggaacagtggtggcaggcaactgatcgcgcaatgaaagctctgggcgatcagcattctctgcaggacgttaaagcattgggtattgccggccagatgcacggagcaaccttgctggatgctcagcaacgggtgttacgccctgccattttgtggaacgacgggcgctgtgcgcaagagtgcactttgctggaagcgcgagttccgcaatcgcgggtgattaccggcaacctgatgatgcccggatttactgcgcctaaattgctatgggttcagcggcatgagccggagatattccgtcaaatcgacaaagtattattaccgaaagattacttgcgtctgcgtatgacgggggagtttgccagcgatatgtctgacgcagctggcaccatgtggctggatgtcgcaaagcgtgactggagtgacgtcatgctgcaggcttgcgacttatctcgtgaccagatgcccgcattatacgaaggcagcgaaattactggtgctttgttacctgaagttgcgaaagcgtggggtatggcgacggtgccagttgtcgcaggcggtggcgacaatgcagctggtgcagttggtgtgggaatggttgatgctaatcaggcaatgttatcgctggggacgtcgggggtctattttgctgtcagcgaagggttcttaagcaagccagaaagcgccgtacatagcttttgccatgcgctaccgcaacgttggcatttaatgtctgtgatgctgagtgcagcgtcgtgtctggattgggccgcgaaattaaccggcctgagcaatgtcccagctttaatcgctgcagctcaacaggctgatgaaagtgccgagccagtttggtttctgccttatctttccggcgagcgtacgccacacaataatccccaggcgaagggggttttctttggtttgactcatcaacatggccccaatgaactggcgcgagcagtgctggaaggcgtgggttatgcgctggcagatggcatggatgtcgtgcatgcctgcggtattaaaccgcaaagtgttacgttgattgggggcggggcgcgtagtgagtactggcgtcagatgctggcggatatcagcggtcagcagctcgattaccgtacggggggggatgtggggccagcactgggcgcagcaaggctggcgcagatcgcggcgaatccagagaaatcgctcattgaattgttgccgcaactaccgttagaacagtcgcatctaccagatgcgcagcgttatgccgcttatcagccacgacgagaaacgttccgtcgcctctatcagcaacttctgccattaatggcgtaaacgttacgcgtcatatgactagttcggacctagggatatcgtcgacatcgatgctcttctgcgttaattaacaattgggatcctctagagttctgtgaaaaacaccgtggggcagtttctgcttcgcggtgttttttatttgtggggcactagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgctcgatttaaatc

SEQ ID NO: 41 - DNA, 10605 bp, Bacillus subtilis
atggctcaatctcattccagttcaatcaactattttggaagcgcaaacaaagtggtttacgaagggaaagattcgactaatcctttagcatttaaatattataatcctcaagaagtaatcggcggaaaaacgctgaaagagcatttgcgattttctattgcctattggcatacatttactgctgatggtacagacgtttttggagcagctacgatgcaaagaccatgggatcactataaaggcatggatctagcgaagatgagagtagaagcagcatttgagatgtttgaaaaactagatgcaccattctttgcttttcatgaccgggatattgcaccagaaggcagtacgctaaaagagacaaaccaaaatttagatatgatcatgggcatgattaaagattacatgagaaatagcggcgttaagctattatggaatacagcaaacatgtttacgaatccccgtttcgtccatggtgccgcgacttcttgcaatgcagatgtgtttgcgtatgctgcagcacaagtgaaaaaagggttagaaacagcaaaagagcttggcgctgagaactatgtattttggggcggccgtgaaggatatgaaacattgttaaataccgatttaaaatttgagcttgatgatttggctagatttatgcatatggcagtggattatgcgaaggaaatcgggtacacagggcagtttttgattgagccaaaaccaaaagagccgaccgcccatcaatacgatacagatgcagcaacaaccattgcctttttgaagcaatatggcttagacaatcattttaaattaaatcttgaagccaatcatgccacattagccgggcatacattcgaacatgaattacgcatggcaagagtacatggtctgcttggctctgttgacgcaaaccagggtcatcctcttttaggctgggacacggatgaatttccgacggatttatattctacgacattagcaatgtacgaaatcctgcaaaatggcggccttggaagcggcggattaaactttgacgcgaaggtcagaagatcttctttcgagcctgatgatctaatatatgcccatattgcagggatggatgcatttgcaagaggattgaaagttgcccacaaattaatcgaagatcgtgtgtttgaagatgtgattcaacatcgttaccgcagctttactgaagggattggtcttgaaattatagaaggaagagctaatttccacacacttgagcaatatgcgctaaatcataaatcaattaaaaacgaatctggaagacaggagaaattaaaagcgatattgaaccaatacattttagaagtataacaggataagctccagatcctgctatcaataccaagtcactgaattacccgtcatgattcctttcctattgcttgttgttatgacgggtaacttctataattaggatttatttagagtgaatggttttttaaaagggcaaggagtgaaaaaatgaagtatgtcattggaatagatcttggaacgagtgctgttaaaaccattttagttaaccaaaacggcaaggtttgtgcagaaacgtccaaaaggtatccgctcatccaagagaaggcgggatatagtgagcaaaatcctgaagactgggttcagcaaacaattgaagcattggctgaattggtttctatatccaatgttcaagccaaggatattgacgggataagctattcgggacaaatgcatggattagtactgcttgaccaagatcgtcaggtgttacgtaatgcaattctttggaatgataccagaacaacgcctcaatgtataaggatgaccgagaaatttggcgatcatcttcttgacatcacaaaaaaccgtgttttagaagggtttacattacctaaaatgttatgggtaaaggaacatgaacctgaactttttaaaaaaactgctgtgtttttgcttccgaaagactacgtgcgattccgtatgaccggtgtcattcacaccgaatactccgatgcagcaggaactttacttttacatattactcgcaaggagtggagcaatgatatttgcaatcaaattggtatttctgcagatatttgtcctccgcttgttgaatctcatgattgtgtaggatcgctgcttccgcacgttgccgcgaagaccgggctattagaaaaaacaaaagtgtacgctgggggagcagataatgcttgcggcgctattggagcaggtatcctttcttccggaaaaacattatgcagtattgggacgtcaggggtcatactttcctacgaagaagaaaaagaaagagactttaaagggaaagtccacttttttaatcatggaaaaaaggattctttttatacgatgggcgtcacgctcgctgcaggatacagcttggactggtttaaaagaacgtttgcaccaaacgaatcgtttgagcaattattgcagggggtggaagctattccgataggagccaatggactgctatacactccttatttggttggtgaaagaacgccgcatgctgattcttctattcggggaagcttgatcggaatggatggagcccataatagaaagcattttttgagggcaataatggaaggtatcacattctctttacatgaatcaattgagctattccgcgaagcgggaaaatcagttcatactgttgtttctattggtgggggagctaaaaatgatacgtggctgcaaatgcaagctgatattttcaatacgagggtaattaagttagaaaatgaacaagggccagctatgggggctgcaatgctggctgcctttggaagcggttggtttgaatcacttgaagaatgtgcagagcagttcattcgtgaggctgctgcattttatccaaaggcgcaaaatgttcaaaaatataaaacactatttgatttgtataagaacatttacactcacacaaaggatctcaatacagctttgaagagctttcgaaaaaactaatcgatttaaatctcgagtagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgaacatcattgccattatgggaccgcatggcgtcttttataaagatgagcccatcaaagaactggagtcggcgctggtggcgcaaggctttcagattatctggccacaaaacagcgttgatttgctgaaatttatcgagcataaccctcgaatttgcggcgtgatttttgactgggatgagtacagtctcgatttatgtagcgatatcaatcagcttaatgaatatctcccgctttatgccttcatcaacacccactcgacgatggatgtcagcgtgcaggatatgcggatggcgctctggttttttgaatatgcgctggggcaggcggaagatatcgccattcgtatgcgtcagtacaccgacgaatatcttgataacattacaccgccgttcacgaaagccttgtttacctacgtcaaagagcggaagtacaccttttgtacgccggggcatatgggcggcaccgcatatcaaaaaagcccggttggctgtctgttttatgattttttcggcgggaatactcttaaggctgatgtctctatttcggtcaccgagcttggttcgttgctcgaccacaccgggccacacctggaagcggaagagtacatcgcgcggacttttggcgcggaacagagttatatcgttaccaacggaacatcgacgtcgaacaaaattgtgggtatgtacgccgcgccatccggcagtacgctgttgatcgaccgcaattgtcataaatcgctggcgcatctgttgatgatgaacgatgtagtgccagtctggctgaaaccgacgcgtaatgcgttggggattcttggtgggatcccgcgccgtgaatttactcgcgacagcatcgaagagaaagtcgctgctaccacgcaagcacaatggccggttcatgcggtgatcaccaactccacctatgatggcttgctctacaacaccgactggatcaaacagacgctggatgtcccgtcgattcacttcgattctgcctgggtgccgtacacccattttcatccgatctaccagggtaaaagtggtatgagcggcgagcgtgttgcgggaaaagtgatcttcgaaacgcaatcgacccacaaaatgctggcggcgttatcgcaggcttcgctgatccacattaaaggcgagtatgacgaagaggcctttaacgaagcctttatgatgcataccaccacctcgcccagttatcccattgttgcttcggttgagacggcggcggcgatgctgcgtggtaatccgggcaaacggctgattaaccgttcagtagaacgagctctgcattttcgcaaagaggtccagcggctgcgggaagagtctgacggttggtttttcgatatctggcaaccgccgcaggtggatgaagccgaatgctggcccgttgcgcctggcgaacagtggcacggctttaacgatgcggatgccgatcatatgtttctcgatccggttaaagtcactattttgacaccggggatggacgagcagggcaatatgagcgaggaggggatcccggcggcgctggtagcaaaattcctcgacgaacgtgggatcgtagtagagaaaaccggcccttataacctgctgtttctctttagtattggcatcgataaaaccaaagcaatgggattattgcgtgggttgacggaattcaaacgctcttacgatctcaacctgcggatcaaaaatatgctacccgatctctatgcagaagatcccgatttctaccgcaatatgcgtattcaggatctggcacaagggatccataagctgattcgtaaacacgatcttcccggtttgatgttgcgggcattcgatactttgccggagatgatcatgacgccacatcaggcatggcaacgacaaattaaaggcgaagtagaaaccattgcgctggaacaactggtcggtagagtatcggcaaatatgatcctgccttatccaccgggcgtaccgctgttgatgcctggagaaatgctgaccaaagagagccgcacagtactcgattttctactgatgctttgttccgtcgggcaacattaccccggttttgaaacggatattcacggcgcgaaacaggacgaagacggcgtttaccgcgtacgagtcctaaaaatggcgggataacttgccagagcggcttccgggcgagtaacgtgctgttaacaaataaaggagacgttatgctgggtttaaaacaggttcaccatattgcgattattgcgacggattatgcggtgagcaaagctttctactgcgatattcttggtttcacgctgcaaagcgaagtctatcgcgaagcgcgcgactcatggaaaggggatttggcgcttaatgggcaatatgtgattgagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

SEQ ID NO: 42 - DNA, 7719 bp, Escherichia coli
tcgatttaaatctcgagtagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcacgggaacaccagcccagcgcgaagaatcaccgccaccgcgctcgccctcaccgccggcgtggccggcagcctcctcgccaccacggcaccggcccaggcgaacatcattgccattatgggaccgcatggcgtcttttataaagatgagcccatcaaagaactggagtcggcgctggtggcgcaaggctttcagattatctggccacaaaacagcgttgatttgctgaaatttatcgagcataaccctcgaatttgcggcgtgatttttgactgggatgagtacagtctcgatttatgtagcgatatcaatcagcttaatgaatatctcccgctttatgccttcatcaacacccactcgacgatggatgtcagcgtgcaggatatgcggatggcgctctggttttttgaatatgcgctggggcaggcggaagatatcgccattcgtatgcgtcagtacaccgacgaatatcttgataacattacaccgccgttcacgaaagccttgtttacctacgtcaaagagcggaagtacaccttttgtacgccggggcatatgggcggcaccgcatatcaaaaaagcccggttggctgtctgttttatgattttttcggcgggaatactcttaaggctgatgtctctatttcggtcaccgagcttggttcgttgctcgaccacaccgggccacacctggaagcggaagagtacatcgcgcggacttttggcgcggaacagagttatatcgttaccaacggaacatcgacgtcgaacaaaattgtgggtatgtacgccgcgccatccggcagtacgctgttgatcgaccgcaattgtcataaatcgctggcgcatctgttgatgatgaacgatgtagtgccagtctggctgaaaccgacgcgtaatgcgttggggattcttggtgggatcccgcgccgtgaatttactcgcgacagcatcgaagagaaagtcgctgctaccacgcaagcacaatggccggttcatgcggtgatcaccaactccacctatgatggcttgctctacaacaccgactggatcaaacagacgctggatgtcccgtcgattcacttcgattctgcctgggtgccgtacacccattttcatccgatctaccagggtaaaagtggtatgagcggcgagcgtgttgcgggaaaagtgatcttcgaaacgcaatcgacccacaaaatgctggcggcgttatcgcaggcttcgctgatccacattaaaggcgagtatgacgaagaggcctttaacgaagcctttatgatgcataccaccacctcgcccagttatcccattgttgcttcggttgagacggcggcggcgatgctgcgtggtaatccgggcaaacggctgattaaccgttcagtagaacgagctctgcattttcgcaaagaggtccagcggctgcgggaagagtctgacggttggtttttcgatatctggcaaccgccgcaggtggatgaagccgaatgctggcccgttgcgcctggcgaacagtggcacggctttaacgatgcggatgccgatcatatgtttctcgatccggttaaagtcactattttgacaccggggatggacgagcagggcaatatgagcgaggaggggatcccggcggcgctggtagcaaaattcctcgacgaacgtgggatcgtagtagagaaaaccggcccttataacctgctgtttctctttagtattggcatcgataaaaccaaagcaatgggattattgcgtgggttgacggaattcaaacgctcttacgatctcaacctgcggatcaaaaatatgctacccgatctctatgcagaagatcccgatttctaccgcaatatgcgtattcaggatctggcacaagggatccataagctgattcgtaaacacgatcttcccggtttgatgttgcgggcattcgatactttgccggagatgatcatgacgccacatcaggcatggcaacgacaaattaaaggcgaagtagaaaccattgcgctggaacaactggtcggtagagtatcggcaaatatgatcctgccttatccaccgggcgtaccgctgttgatgcctggagaaatgctgaccaaagagagccgcacagtactcgattttctactgatgctttgttccgtcgggcaacattaccccggttttgaaacggatattcacggcgcgaaacaggacgaagacggcgtttaccgcgtacgagtcctaaaaatggcgggataacttgccagagcggcttccgggcgagtaacgtgctgttaacaaataaaggagacgttatgctgggtttaaaacaggttcaccatattgcgattattgcgacggattatgcggtgagcaaagctttctactgcgatattcttggtttcacgctgcaaagcgaagtctatcgcgaagcgcgcgactcatggaaaggggatttggcgcttaatgggcaatatgtgattgagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc